(19) United States
(12) Patent Application Publication
(10) Pub. No.: US 2014/0148576 A1
(43) Pub. Date: May 29, 2014

(54) Traceless Ubiquitination

(71) Applicants: Jason Chin, Cambridge (GB); Satpal Virdee, Dundee (GB)

(72) Inventors: Jason Chin, Cambridge (GB); Satpal Virdee, Dundee (GB)

(73) Assignee: Medical Research Council, Wiltshire (GB)

(21) Appl. No.: 14/129,028

(22) PCT Filed: Jun. 22, 2012
(86) PCT No.: PCT/GB2012/000543

§ 371 Date: Dec. 23, 2013

(30) Foreign Application Priority Data
Jun. 24, 2011 (GB) 1110815.6

(51) Int. Cl. C12P 21/00; C07C 271/22; C07C 323/59; C12N 9/10

(52) U.S. Cl. CPC C12P 21/00; C12N 9/104; C07C 271/22; C07C 323/59
USPC 530/350; 435/193; 536/23.6; 435/68.1; 560/159; 435/320.1; 435/252.33

(57) ABSTRACT

The invention related to a tRNA synthetase capable of binding delta-substituted lysine, wherein said tRNA synthetase comprises amino acid sequence corresponding to the amino acid sequence of at least L271 to Y349 of MbPyIRS, wherein said sequence comprises 5 or fewer substitutions within the amino acid sequence corresponding to the amino acid sequence of at least L271 to Y349 of MbPyIRS; and wherein said synthetase comprises W at amino acid position 349 relative to MbPyIRS.

### FIELD OF THE INVENTION

The invention relates to incorporation of substituted lysines into polypeptides. In particular the invention relates to incorporation of delta-substituted lysines.

### BACKGROUND TO THE INVENTION

The site-specific addition of ubiquitin to proteins is a post-translational modification that regulates almost all aspects of eukaryotic biology 1.2. In the process of ubiquitination the epsilon amino group of a lysine residue within the substrate protein is linked to the C-terminal carboxylate of ubiquitin (a 76 amino acid protein) via an isopeptide bond. In vivo, ubiquitin is attached to its substrates by a series of enzymes (E1s, E2s, E3s) that direct isopeptide bond formation. Studying the molecular consequences of protein ubiquitination is challenging, since there are >600 E3 ubiquitin ligases believed to be responsible for substrate recognition 3, and the E3 ligase for specific substrates are often unknown. Moreover, even when the ligases are known they may not drive the reaction to completion or at a unique site in vitro.

Several investigators hove addressed the creation of ubiquitin conjugates that are connected via non-native linkages including: a disulphide bond 4,5, an oxime 6, triazoles 7, and an isopeptide bond in which the universally conserved C-terminal glycine of ubiquitin is mutated to D-cysteine 8, or alanine 9 in a non-traceless native chemical ligation. While some of these non-native linkages have found utility 4,5,9 a clear and important challenge is to address the creation of methods for the ubiquitination of any protein, at a user-defined site via an entirely native isopeptide bond.

We recently described a new approach, in protein chemistry termed GOPAL, for creating native isopeptide bonds between ubiquitin and a specific lysine in a target protein 10. Using the M. barkeri (Mb) pyrrolysyt-tRNA synthetase (PyIRS)/tRNACUA pair, which naturally introduces pyrrolysine (1) into proteins in certain methanogens, we site specifically inserted Nε-(t-butyoxycarbonyl)-L-lysine (2) into ubiquitin and developed a series of selective protection and deprotection steps that allowed us to direct site-selective isopeptide bond formation. Using this approach we were able to generate important ubiquitin dimers linked through specific isopeptide bonds, solve the crystal structure of Lys6-linked diubiquitin and reveal new deubiquitinase specificity. Since this method relies on cellular protein synthesis to generate the component proteins it is in principle scalable to creating traceless isopeptide bonds between proteins of any size. However this method does require multiple protection and deprotection steps, generating highly protected hydrophobic intermediates that con be poorly soluble. Moreover the method may be challenging to apply to proteins that cannot be refolded.

Four distinct lysine derivatives (3-6) that can be incorporated into synthetic peptides via solid phase peptide synthesis methods have been described 11-17. 3 can be ligated with C-terminal thioesters of ubiquitin, and subsequent auxiliary removal allows the generation of a native isopeptide bond 11,12. The deprotection of 4-6 allows their ligation with C-terminal thioesters and subsequent desulfurization yields native isopeptide bonds 13-16. Unfortunately, while these amino acids can be incorporated into longer peptides via rounds of native chemical ligation with thioesters these steps require further thiol protection, decrease the yield of protein conjugates and ultimately limit the length of proteins, and/or the positions within proteins to which these approaches might be applied.

The present invention seeks to overcome problem(s) associated with the prior art.

### SUMMARY OF THE INVENTION

In one aspect the invention provides a tRNA synthetase capable of binding delta-substituted lysine,

wherein said tRNA synthetase comprises amino acid sequence corresponding to the amino acid sequence of at least L271 to Y349 of MbPyIRS.
wherein said sequence comprises 5 or fewer substitutions within the amino acid sequence corresponding to the amino acid sequence of at least L271 to Y349 of MbPyIRS; and
wherein said synthetase comprises W at amino acid position 349 relative to MbPyIRS.

Suitably the tRNA synthetase comprises N at position 311.

Suitably the tRNA synthetase further comprises a mutation relative to the wild type MbPyIRS sequence at one or more of Y271, L274 and C313.

Suitably the tRNA synthetase comprises Y271M, L274G and C313A.

In another aspect, the invention relates to a nucleic acid comprising nucleotide sequence encoding a tRNA synthetase according to any of claims 1 to 4.

In another aspect, the invention relates to use of a tRNA synthetase according to any of claims 1 to 4 to charge a tRNA with a delta-substituted lysine.

Suitably said tRNA comprises MbtRNA_{CUA}.

In another aspect, the invention relates to a method of making a polypeptide comprising delta-substituted lysine comprising arranging for the translation of a RNA encoding said polypeptide, wherein said RNA comprises an orthogonal codon, wherein said translation is carried out in the presence of a tRNA synthetase according to any of claims 1 to 4 and in the presence of tRNA which recognises the orthogonal codon and is capable of being charged with delta-substituted lysine, and in the presence of delta-substituted lysine.

Suitably the orthogonal codon is the amber codon (TAG).

Suitably the delta-substituted lysine is also epsilon substituted.

Suitably the delta-substituted lysine is selected from the group consisting of 9, 10, 13, and 14.

Suitably the delta-substituted lysine is 9 or 10 and wherein the method further comprises the step of removing the butyloxycarbonyl (boc) group.

Suitably the step of removing the butyloxycarbonyl (boc) group comprises contacting the polypeptide with 60% trifluoroacetic acid (TFA) at 22° C. for 1 hour.

Suitably the delta-substituted lysine is 13 or 14 and wherein the method further comprises the step of removing the nitrocarbylbenzyloxy (nitroCbz) group.

Suitably the step of removing the nitrocarbyfbenzyloxy (nitroCbz) group comprises reducing the aromatic nitro group to online and fragmenting the aniline to reveol the free epsilon amino group.

Suitably the step of removing the nitrocarbylbenzyloxy (nitroCbz) group comprises performing one-fix-elimination.

In another aspect, the invention relates to a method of incorporating a ubiquitin-like modifier into a polypeptide comprising

(a) incorporating a delta-substituted lysine into a polypeptide as described above; and

(b) ligating said ubiquitin-like modifier to the delta-substituted lysine of (a).

Suitably the ubiquitin-like modifier comprises ubiquitin. SUMO, ISG15, Nedd, FAT10, Ufm1 or ATG12.

Suitably the ubiquitin-like modifier comprises ubiqutin, sumo, ISG or Nedd.

Suitably ubiquitin-like modifier comprises ubiquitin.

In another aspect, the invention relates to a delta-substituted lysine selected from the group consisting of 9, 10, 11, 12, 13, 14.

In another aspect, the invention relates to a polypeptide comprising a delta-substituted lysine as described above.

Suitably the lysine is an isotopically labelled lysine.

In another aspect, the invention relates to a vector comprising nucleic acid as described above.

Suitably said vector further comprises nucleic acid sequence encoding a tRNA substrate of said tRNA synthetase.

Suitably said tRNA substrate is encoded by the MbPyIT gene.

In another aspect, the invention relates to a cell comprising a nucleic acid as described above, or comprising a vector as described above.

In another aspect, the invention relates to a kit comprising

(i) a vector as described above

(I) a delta substituted lysine selected from the group consisting of 9, 10, 13 and 14.

Suitably the kit further comprises

(iii) a vector comprising sequence encoding the MbPyIT tRNA.

Suitably the vector of (iii) further comprises a cloning site to accept nucleic acid sequence encoding the target polypeptide and further comprises nucleic acid elements capable of directing expression of said target polypeptide.

### DETAILED DESCRIPTION OF THE INVENTION

Protein ubiquitination is a post-translational modification that regulates almost all aspects of eukaryotic biology. We have discovered the first routes for the efficient site-specific incorporation of δ-thiol-L-lysine (7) and δ-hydroxy-L-lysine (8) into recombinant proteins, and combined the genetically directed incorporation of 7 with native chemical ligation and desulfurization to yield an entirely native isopeptide bond between substrate proteins and ubiquitin

The inventors realised that an ideal, scalable and traceless route to creating site-specific isopeptide bonds would combine genetic code expansion and native chemical ligation (FIG. 1). A lysine derivative for traceless native chemical ligation could be site-specifically incorporated into an overexpressed protein using the cells protein translation machinery. The protein could then be purified and used. In combination with a ubiquitin thioester prepared by intein fusion thiolysis, to direct the synthesis of ubiquitin conjugates linked via on entirely native isopeptide bond. This would provide a simple, scaleable and broadly accessible route to ubiquitinated proteins. We were particularly interested in incorporating δ-thiol-L-lysine (7) since this has recently been used in peptide ligation at several sites. 16,17 This suggests that native chemical lgation using this amino acids may work well at a range of sites in diverse proteins. In the process of this work we also incorporated another important post-translational modification, δ-hydroxy lysine (8).

Since a simple δ-thiol lysine differs from lysine only by the insertion of a sulfur atom it may be thermodynamically challenging to create a synthetase that will recognize the thiol compound but exclude lysine by a factor of 103-104, as required to maintain the fidelity of natural protein translation. t-butyloxycarbonyl protected lysine (2) is a good substrate for PyRS 18,19, and we have previously demonstrated that while the PyIRS/tRNACUA pair does not selectively incorporate Ne-methyl-L lysine it can accommodate on Ne methyl derivative of lysine, which also bears the Ne-t-butyloxycarbonyl (boc) group 19. Since the Boc group can be removed after incorporation of the amino acid into the protein, this provides a paradigm for installing modifications on the epsilon amino group that cannot be installed directly. We therefore investigated whether the addition of an Ne t-butyloxycarbonyl group will also facilitate the incorporation of δ substituted lysine derivatives 19,10).

### Advantages

The targeted approach provided by the present invention has the advantage of avoiding incorrect or undesired bonding.

Prior art methods for modifying polypeptides have tended to involve very numerous protecting groups on the residues being targeted. The presence of very numerous protecting groups on polypeptides typically leads to problems with solubility, and can make such polypeptides very difficult to work with. The present invention advantageously reduces or eliminates the use of protecting groups.

The conditions for chemical ligation to polypeptides can be highly protein specific. Equally, the chemical conditions for removal of protection groups can also be very protein specific. Similarly, the conditions for refolding of a denatured or partially denatured polypeptide can also be protein specific. The present invention advantageously avoids or reduces the need for these chemical manipulations. Consequently, the chemical treatment of polypeptides according to the invention is considerably simplified.

It is an advantage of the invention that the unnatural amino acid(s) incorporated may be used to drive(s) a selective chemical reaction. This selectivity has the further advantage of further reducing or removing the need for chemical protection of the reactive groups.

It is an advantage of the invention that the reactions described can be conducted in aqueous solutions.

It is an advantage of the invention that the reaction chemistries described can be performed on folded proteins. In other words, the use of chaotropes (which is very often required in prior art techniques) to unfold proteins for chemical modification can be advantageously reduced or avoided.

### Applications

The invention is illustrated with reference to ubiquitination. In particular the examples section features numerous reactions involving the addition of ubiquitin to polypeptide chains. These are exemplary in nature, since the invention may equally be applied to other (non-ubiquitin) modifications of polypeptides. Indeed the invention may be applied to joining of the polypeptide comprising the delta substituted lysine to any further polypeptide that can form an isopeptide bond with a lysine residue.

More specifically, suitably the invention may be used for incorporation of ubiquitin-ike modifiers into polypeptides. Examples of ubiquitin-like modifiers include SUMO, ISG15, Nedd (e.g. Nedd8), FAT10, Ufm1 and ATG12 as well as ubiquitin.

Suitably, the invention may be used with SUMO in order to sumoylate polypeptides.

Suitably, the invention may be used with ISG15 in order to ISGylate polypeptides.

The chemical manipulations and reaction conditions are illustrated with reference to ubiquitination. In outline, the reaction conditions for other modifications are the some as for ubiquitin. For example, the group to be added such as ubiquitin may be activated. This may be performed by creating a thioester group as the reactive species for joining to the polypeptide of interest.

Systems for producing activated moieties for addition to the polypeptides are commercially available. One such example is by use of on intein fusion to the polypeptide which is to be joined to the polypeptide of interest. For example, New England BioLabs Inc. sell an intein fusion kit which may be employed to produce activated moieties for joining to the polypeptide of interest according to the present invention. Suitably, the intein fusion kit is used according to the manufacturer's instructions.

Production of an activated SUMO (SUMO thioester) is described for example in Chatterjee et al (Angewandte Chemie 2007 vol 46 pages 2814-2818). This document is incorporated specificaly for the method of production of SUMO thioester.

Production of on activated ISG15 (ISG15 thioester) is described for example in Akutsu et al (PNAS 2010 “Molecular basis for ubiquitin and ISG15 cross-reactivity in viral ovarian tumour domains”). This document is incorporated specifically for the method of production of ISG15 thioester.

When it is desired to add a moiety other than ubiquitin to the polypeptide of interest, then the moiety is simply substituted for ubiquitin according to the illustrations presented herein. For example, for sumoylotion, the SUMO polypeptide is the moiety for joining to the polypeptide of interest; the SUMO amino acid sequence is simply substituted for the ubiquitin amino acid sequence. For example, when the moiety to be joined to the polypeptide of interest is ISG15, the amino acid sequence of ISG15 is simply substituted for the amino acid sequence of ubiquitin in the methods described therein. This applies equally for other moieties to be joined to the polypeptide of interest. These other moieties are typically referred to as “ubiquitin like modifiers”. Suitably ubiquitin like modifiers share the common property of all forming in isopeptide bond as the point of joining to the polypeptide of interest.

An alternative technique for joining to the unnatural amino acid incorporated into the polypeptide of interest according to the present invention is to simply make the moiety to be joined as a synthetic thioester, and then react this thioester compound directly with polypeptide of interest produced according to the present invention. For example, in the case of ISGylation, an ISG thioester would be manufactured synthetically, and this ISG thioester would then be reacted with a delta substituted lysine reside incorporated into the polypeptide of interest as described herein.

### Substituted Lysines

The invention relates to the incorporation of a delta substituted lysines into polypeptides. It is believed that this is the first disclosure of incorporation of delta substituted lysines into polypeptides.

Suitably any delta substituted lysine is incorporated. Suitably the delta substituted lysine is selected from the group consisting of 5, 9, 10, 12, 13, and 14. Suitably the delta substituted lysine is selected from the group consisting of 9, 10, 12, 13 and 14. Suitably the delta substituted lysine is 9 or 10. Suitably the delta substituted lysine is 12 or 13 or 14.

Suitably the delta substitution comprises an atom from group 6 of the periodic table. Suitably the delta substitution comprises oxygen, sulphur or selenium. Most suitably the substitution comprises hydroxyl (OH), thiol (SH) or selenol (SeH).

When the invention is applied to incorporation of a selenium derivative, suitably said selenium derivative is in the form of a latent selenol such as a selenozolidine for example as in B below:

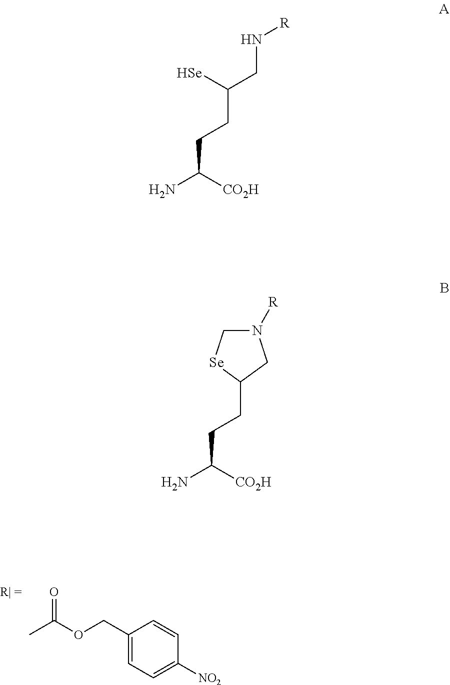

It may be possible to incorporate a selenium derivative bearing selenium as a free selenol (as shown in A above). However, this may be less desirable since this form may require careful handling due to increased reactivity. For this reason, when the derivative comprises selenium, suitably said derivative is a selenozolidne amino acid such as B above.

When the delta substitution is selenol, this has the advantage of avoiding a desulphurisation reaction. This has the further advantage of being more reactive. Being more reactive provides the benefit of being able to use milder chemical conditions for joining to the delta substituted position of the lysine.

Desulphurisation of a polypeptide risks converting cysteines in the polypeptide to olanines. Thus, suitably polypeptides comprising cysteine are not subjected to a desulphurisation reaction. Suitably the polypeptide of interest does not comprise cysteine.

The chemical group present at the delta substituted site of the lysine is suitably of a small molecular size. For example, suitably the chemical group present has the delta substitution is smaller than the methyl disulphide of 11.

tRNA Synlhelase

Suitably the tRNA synthetase of the invention has a substitution of the naturally occurring tyrosine (Yj residue at position 349 of the wild type sequence for tryptophan (W). In other words, suitably the tRNA synthetase of the invention has a Y349W mutation. This mutation is important because it provides the molecular space within the active site of the tRNA synthetase which accommodates a chemical group which is present as the delta substitution. Examples of the chemical group which may be present as the delta substitution include —OH, —SH, —SeH.

Suitably, the tRNA synthetase used to incorporate a delta substituted lysine comprises the Y349W mutation.

Further mutations may be comprised by the tRNA synthetase used. For example, we demonstrate incorporation of delta substituted lysines which comprise a further substitution at the epsilon position. Examples of these are nitroCbz substituted lysines, for example, 12, 13 and 14 as shown herein. Mutations which are already known to accommodate chemical groups at alternate substitution positions within the lysine may be included into the synthetase used for incorporation of the delta substituted lysine of the invention. For example, the tRNA synthetase of the invention may further comprise mutations at position Y271, L274 and C313. In particular, the tRNA synthetase of the invention may comprise of Y271M, L274G and C313A.

Without wishing to be bound by theory, it is believed that accommodative properties of these extra tRNA synthetase mutations are additive. In other words, in order to render the tRNA synthetase permissive of inclusion of an epsilon substituted lysine those residues important for accommodating epsilon substitutions should also be used in the tRNA synthetase. Thus, so long as the Y349W mutation is included in the synthetase which is used to incorporate delta substituted lysine into the polypeptide of interest, other mutations may also be present as desired by the operator.

It should be noted that some of the delta substituted lysines may be too similar to naturally occurring lysine to be adequately discriminated by the tRNA synthetases herein such as the Y349W mutant. For example, delta thiol lysine (7) and delta hydroxyl lysine (8) may not be directly incorporated into the polypeptide of interest using the tRNA synthetases described. However, these moieties can be effectively incorporated into the polypeptide of interest by instead incorporating 13 (to produce hydroxyl lysine (8) or 14 (to produce thiol lysine 7). By way of explanation, incorporation of the smaller 7 or 8 from the larger 14 or 13 results from the translational incorporation of 14 or 13 into the polypeptide of interest, and the subsequent removal of the p-nitroCbz group from the polypeptide.

The nitroCbz groups may be removed from the polypeptide by any suitable method known in the art. For example, they may be removed by reduction to amine using sodium dithionite. This reaction may sometimes be referred to as “one fix elmination”. For example, the reaction may proceed by deprotection of the p-nitrocarbobenzyloxy group under mild conditions using sodium dithionite. An example of this is described in Dreef-Tromp et al 1992 (NAR vol 20 pages 4015-4020). This document is incorporated specifically for the method of deprotection.

Alternatively, it may not be necessary to use a specific chemical reaction to remove the nitroCbz groups. For example, we demonstrate their removal herein as part of the purification process. Without wishing to be bound by theory, it appears that the nitroCbz groups may be removed by naturally occurring host factors which contact the polypeptide during lysis of the cells and recovering of the purified polypeptide of interest. This is occasionally referred to as “automatic deprotection”. This has the advantage of avoiding chemical deprotection and/or light treatment in order to remove the nitroCbz groups.

Whatever changes may be made to the tRNA synthetase, suitably it always possesses the Y349W mutation.

It is further disclosed that residue 311 is important to the incorporation of substituted lysines. In the wild type synthetase, position 311 is asparagine(N). Suitably the synthetase used in the present invention retains the wild type N311. Suitably the synthetase used in the present invention does not comprise any mutation at position 311. Without wishing to be bound by theory, it is believed that mutations at position 311 lead to the incorporation of different naturally occurring amino acids. This leads to a heterogeneous polypeptide product, which is disadvantageous. Thus, although it may be possible to use synthetases having a mutation at position 311, this would be undesirable since it would require further purification in order to separate the desired polypeptides from those having undesired amino ocids at the target site.

### DEFINITIONS

The term ‘comprises’ (comprise, comprising) should be understood to have its normal meaning in the art, i.e. that the stated feature or group of features is included, but that the term does not exclude any other stated feature or group of features from also being present.

The Invention makes use of orthogonal tRNA synthetase-orthogonal tRNA pairs that can process information in parallel with wild-type tRNA synthetases and tRNAs but that do not engage in cross-talk between the wild-type and orthogonal molecules. In some embodiments the tRNA itself may retain its wild type sequence. In those embodiments, suitably said entity retaining its wild type sequence is used in a heterologous setting i.e. in a background or host cell different from its naturally occurring wild type host cell. In this way, the wild type entity may be orthogonal in a functional sense without needing to be structurally altered. Orthogonality and the accepted criteria for same are discussed in more detail below.

The Methonosarcina barkeri PyIS gene encodes the MbPyIRS tRNA synthetase protein. The Methonosarcina barked PyIT gene encodes the MbtRNA_{CUA} tRNA.

### Sequence Homology/Identity

Although sequence homology can also be considered in terms of functional similarity (i.e., amino acid residues having similar chemical properties/functions), in the context of the present document it is preferred to express homology in terms of sequence identity.

Sequence comparisons can be conducted by eye or, more usually, with the aid of readily available sequence comparison programs. These publicly and commercially available computer programs can calculate percent homology (such as percent identity) between two or more sequences.

Percent identity may be calculated over contiguous sequences, i.e., one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example in an otherwise identical pair of sequences, one insertion or deletion will cause the following amino acid residues to be put out of alignment, thus potentially resulting in a large reduction in percent homology (percent identity) when a global alignment (an alignment across the whole sequence) is performed. Consequently, most sequence comparison methods are designed to produce optimal alignments that take into consideration possible insertions and deletions without penalising unduly the overall homology (identity) score. This is achieved by inserting “gaps” in the sequence alignment to try to maximise local homology/identity.

These more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typicaly used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum percent homology therefore firstly requires the production of an optimal alignment, taking into consideration gap penalties. A suitable computer program for carrying out such on alignment is the GCG Wisconsin Bestfit package (University of Wisconsin, U.S.A; Devereux et al., 1984, Nucleic Acids Research 12:387).

Examples of other software than can perform sequence comparisons include, but are not limited to, the BLAST package, FASTA (Altschul et al., 1990, J. Mol. Biol. 215:403-410) and the GENEWORKS suite of comparison tools.

Although the final percent homology can be measured in terms of identity, the alignment process itself is typically not based on an all-or-nothing pair comparison. instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the pubic default values or a custom symbol comparison table if supplied. It is preferred to use the public default values for the GCG package, or in the case of other software, the default matrix, such as BLOSUM62. Once the software has produced an optimal alignment, it is possible to calculate percent homology, preferably percent sequence identity. The software typically does this as port of the sequence comparison and generates a numerical result.

In the context of the present document, a homologous amino acid sequence is taken to include an amino acid sequence which is at least 15, 20, 25, 30, 40, 50, 60, 70, 80 or 90% identical, preferably at least 95 or 98% identical at the amino acid level. Suitably this identity is assessed over at least 50 or 100, preferably 200, 300, or even more amino acids with the relevant polypeptide sequence(s) disclosed herein, most suitably with the ful length progenitor (parent) tRNA synthetase sequence. Suitably, homology should be considered with respect to one or more of those regions of the sequence known to be essential for protein function rather than non-essential neighbouring sequences. This is especially important when considering homologous sequences from distantly related organisms.

Most suitably sequence identity should be judged across at least the contiguous region from L271 to Y349 of the amino acid sequence of MbPyIRS, or the corresponding region in on alternate tRNA synthetase.

Most suitably the synthetase of the invention comprises an amino acid sequence having at least 93.5% identity to the contiguous region from L271 to Y349 of the amino acid sequence of MbPyIRS.

Most suitably the synthetase of the invention comprises on amino acid sequence having 5 or fewer substitutions relative to the contiguous region from L271 to Y349 of the amino acid sequence of MbPyIRS.

Most suitably the synthetase of the invention comprises an amino acid sequence having at least 94.8% identity to the contiguous region from L271 to Y349 of the amino acid sequence of MbPyIRS.

Most suitably the synthetase of the invention comprises an amino acid sequence having 4 or fewer substitutions relative to the contiguous region from L271 to Y349 of the amino acid sequence of MbPyIRS.

Most suitably the synthetase of the invention comprises an amino acid sequence having at least 96.1% identity to the contiguous region from L271 to Y349 of the amino acid sequence of MbPyIRS.

Most suitably the synthetase of the invention comprises an amino acid sequence having 3 or fewer substitutions relative to the contiguous region from L271 to Y349 of the amino acid sequence of MbPyIRS.

Most suitably the synthetase of the invention comprises an amino acid sequence having at least 97.4% identity to the contiguous region from L271 to Y349 of the amino acid sequence of MbPyIRS.

Most suitably the synthetase of the invention comprises an amino acid sequence having 2 or fewer substitutions relative to the contiguous region from L271 to Y349 of the amino acid sequence of MbPyRS.

Most suitably the synthetase of the invention comprises an omino acid sequence having at least 98.7% identity to the contiguous region from L271 to Y349 of the amino acid sequence of MbPyIRS.

Most suitably the synthetase of the invention comprises on amino acid sequence having 1 substitution relative to the contiguous region from L271 to Y349 of the amino acid sequence of MbPyIRS. In this embodiment suitably the one substitution is suitably Y349W.

Suitably the tRNA synthetase of the invention always possesses at least the Y349W substitution relative to the amino acid sequence of MbPyIRS.

Regions outside this domain may be mutated at the desire of the operator, always ensuring that the appropriate tRNA charging (aminoacylation) function is retained. This tRNA charging function can be easily checked according to the techniques noted herein.

The same considerations apply to nucleic acid nucleotide sequences, such as (RNA sequence(s).

### Reference Sequence

When particular amino acid residues are referred to using numeric addresses, the numbering is taken using MbPyIRS (Methonosarcina barkeri pyrrolysyl-tRNA synthetase) amino acid sequence as the reference sequence (i.e. as encoded by the publicly available wild type Methonosarcina barked PyIS gene Accession number Q46E77):

MDKKPLDVLI SATGLWNSRT GTLHKIKHYE VSRSKIYIEM ACGDHLVVNN SRSCRTARAF
RHHKYRKTCK RCRVSDEDIN NFLTRSTEGK TSVKVKVVSA PKVKKAMPKS VSRAPKPLEN
PVSAKASTDT SRSVPSPAKS TPNSPVPTSA PAPSLTRSQL DRVEALLSPE DKISLNIAKP
FRELESELVT RRKNDFQRLY TNDREDYLGK LERDITKFFV DRDFLEIKSP ILIPAEYVER
MGINNDTELS KQIFRVDKNL CLRPMLAPTL YNYLRKLDRI LPDPIKIFEV GPCYRKESDG
KEHLEEFTMV NFCQMGSGCT RENLESLIKE FLDYLEIDFE IVGDSCMVYG DTLDIMHGDL
ELSSAVVGPV PLDREWGIDK PWIGAGFGLE RLLKVMHGFK NIKRASRSES YYNGISTNL

This is to be used as is well understood in the art to locate the residue of interest. This is not always a strict counting exercise—attention must be paid to the context. For example, if the protein of interest is of a slightly different length, then location of the correct residue in that sequence corresponding to (for example) Y349 may require the sequences to be aligned and the equivalent or corresponding residue picked, rather than simply taking the 349^{th} residue of the sequence of interest. This is well within the ambit of the skilled reader.

Mutating has it normal meaning in the art and may refer to the substitution or truncation or deletion of the residue, motif or domain referred to. Mutation may be effected at the polypeptide level e.g. by synthesis of a polypeptide having the mutated sequence, or may be effected at the nucleotide level e.g. by making a nucleic acid encoding the mutated sequence, which nucleic acid may be subsequently translated to produce the mutated polypeptide. Where no amino acid is specified as the replacement amino acid for a given mutation site, suitably a randomisation of said site is used, for example as described herein in connection with the evolution and adaptation of tRNA synthetase of the invention. As a default mutation, alanine (A) may be used. Suitably the mutations used at particular site(s) are as set out herein.

Thus a Y349W mutant is produced from the wild type sequence by changing Y to W at the position corresponding to Y349: using to illustrate this a Y349W polypeptide would have the sequence:

MDKKPLDVLI SATGLWNSRT GTLHKIKHYE VSRSKIYIEM ACGDHLVVNN SRSCRTARAF
RHHKYRKTCK RCRVSDEDIN NFLTRSTEGK TSVKVKVVSA PKVKKAMPKS VSRAPKPLEN
PVSAKASTDT SRSVPSPAKS TPNSPVPTSA PAPSLTRSQL DRVEALLSPE DKISLNIAKP
FRELESELVT RRKNDFQRLY TNDREDYLGK LERDITKFFV DRDFLEIKSP ILIPAEYVER
MGINNDTELS KQIFRVDKNL CLRPMLAPTL YNYLRKLDRI LPDPIKIFEV GPCYRKESDG
KEHLEEFTMV NFCQMGSGCT RENLESLIKE FLDYLEIDFE IVGDSCMVWG DTLDIMHGDL
ELSSAVVGPV PLDREWGIDK PWIGAGFGLE RLLKVMHGFK NIKRASRSES YYNGISTNL

This applies equally to each of the other mutations discussed herein.

A fragment is suitably at least 10 amino acids in length, suitably at least 25 amino acids, suitably at least 50 amino acids, suitably at least 100 amino acids, suitably at least 200 amino acids, suitably at least 250 amino acids, suitably at least 300 amino acids, suitably at least 349 amino acids, or suitably the majority of the tRNA synthetase polypeptide of interest.

Suitably polypeptides of the invention are manufactured by causing expression of a nucleotide sequence encoding them, for example in a suitable host cell.

Nucleotide sequences of the invention are suitably those encoding the polypeptides of the invention.

An exemplary nucleotide sequence is produced by mutating the sequence encoding wild type Methonosarcina barker PyIS polypeptide, which sequence is:

atggataaaaaaccattagatgttttaatatctgcgaccgggctctggat
gtccaggactggcacgctccacaaaatcaaacactatgaggtctcaagaa
gtaaaatatacattgaaatggcgtgtggagaccatcttgttgtgaataat
tctaggagttgtagaacagccagagcattcagacatcataagtacagaaa
aacctgcaaacgatgtagggtttcggacgaggatatcaataatttcctca
caagatcaactgaaggcaaaaccagtgtgaaagttaaggtagtttctgct
ccaaaggtcaaaaaagctatgccgaaatcagtttcgagggctccaaagcc
tctggaaaatcctgtgtctgcaaaggcatcaacggacacatccagatctg
taccttcgcctgcaaaatcaactccaaattcgcctgttcccacatcggct
cctgctccttcacttacaagaagccagctcgatagggttgaggctctctt
aagtccagaggataaaatttctctgaatattgcaaagcctttcagggaac
ttgagtccgaacttgtgacaagaagaaaaaacgattttcagcggctctat
accaatgatagagaagactaccttggtaaactcgaacgggacattacgaa
atttttcgtagaccgggattttctggagataaagtctcctatccttattc
cggcagaatacgtggagagaatgggtattaacaatgatactgaactttca
aaacagatcttcagggtggataaaaatctctgcttaaggccaatgcttgc
cccgactctttacaactatctgcgaaaactcgataggattttaccagatc
ctataaagattttcgaagtcgggccctgttaccggaaagagtctgacggc
aaagagcacctggaagaatttaccatggtgaacttctgtcagatgggttc
gggatgtactcgggaaaatcttgaatccctcatcaaagagtttctggact
atctggaaatcgacttcgaaatcgtaggagattcctgtatggtctatggg
gatacccttgatataatgcacggggacctggagctttcttcggcagtcgt
cgggccagttcctcttgatagggaatggggcattgacaaaccatggatag
gtgcaggttttgggcttgaacgcttgctcaaggttatgcatggctttaaa
aacattaagagagcatcaaggtccgaatcttactataatgggatttcaac
caatctatga

to change the codon for Y349 to a codon for W.

This can be accomplished by any suitable means known in the art such as site directed mutagenesis, PCR, synthesis of oligonucleotides (with ligation and sequencing as necessary) or other suitable method.

This applies equally to each of the other mutations discussed herein.

Polynucleotides of the invention can be incorporated into a recombinant replicable vector. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells include bacteria such as E. coli.

Preferably, a polynucleotide of the invention in a vector is operably linked to a control sequence that is capable of providing for the expression of the coding sequence by the host cell, i.e. the vector is an expression vector. The term “operably inked” means that the components described are in a relationship permitting them to function in their intended manner. A regulatory sequence “operably linked” to a coding sequence is ligated in such a way that expression of the coding sequence is achieved under condition compatible with the control sequences.

Vectors of the invention may be transformed or transfected into a suitable host cell as described to provide for expression of a protein of the invention. This process may comprise culturing a host cell transformed with on expression vector as described above under conditions to provide for expression by the vector of a coding sequence encoding the protein, and optionally recovering the expressed protein.

The vectors may be for example, plasmid or virus vectors provided with on origin of replication, optionally a promoter for the expression of the said polynucleotide and optionally a regulator of the promoter. The vectors may contain one or more selectable marker genes, for example an ampicillin resistance gene in the case of a bacterial plasmid. Vectors may be used, for example, to transfect or transform a host cell.

Control sequences operably linked to sequences encoding the protein of the invention include promoters/enhancers and other expression regulation signals. These control sequences may be selected to be compatible with the host cell for which the expression vector is designed to be used in. The term promoter is well-known in the art and encompasses nucleic acid regions ranging in size and complexity from minimal promoters to promoters including upstream elements and enhancers.

### Protein Expression and Purification

Host cells comprising polynucleotides of the invention may be used to express proteins of the invention. Host cells may be cultured under suitable conditions which allow expression of the proteins of the invention. Expression of the proteins of the invention may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

Proteins of the invention can be extracted from host cells by a variety of techniques known in the art, including enzymatic, chemical and/or osmotic lysis and physical disruption.

### Optimisation

Unnatural amino acid incorporation in in vitro translation reactions can be increased by using S30 extracts containing a thermally inactivated mutant of RF-1. Temperature sensitive mutants of RF-1 allow transient increases in global amber suppression in vivo. Increases in IRNA_{CUA} gene copy number and a transition from minimal to rich media may also provide improvement in the yield of proteins incorporating on unnatural amino acid in E. coli.

tRNA Synthetases

The tRNA synthetase of the invention may be varied. Although specific tRNA synthetase sequences may have been used in the examples, the invention is not intended to be confined only to those examples.

In principle any tRNA synthetase which provides the some tRNA charging (aminoacylation) function can be employed in the invention. In other words any tRNA synthetase capable of incorporating delta-substituted lysine may be used in the invention.

For example the tRNA synthetase may be from any suitable species such as from archea, for example from Methanosarcina barkeri MS; Methanosarcina barkeri str. Fusaro; Methanosarcino mozei GoI; Methonosarcino ocetivorons C2A: Methanosarcino thermophila; or Methanococcoides burtonii. Alternatively the tRNA synthetase may be from bacteria, for example from Desultitobacterium hafniense DCB-2; Desulfitobocterium hafniense Y5; Desulfitobocterium hafniense PCP1: Desulfotomaculum ocetoxidons DSM 771.

Exemplary sequences from these organisms are the publically available sequences. The following examples are provided as exemplary sequences for pyrrolysine tRNA synthetases:

>M.barkeriMS/1-419/
Methanosarcina barkeri MS
VERSION 16WRH6.1 GI:74501411
MDKKPLDVLISATGLWMSRTGTLHKIKHHEVSRSKIYIEMACGDHLVVNNSRSCRTARAFRHHKYRKTC
KRCRVSDEDINNFLTRDTESKNSVKVRVVSAPKVKKAMPKSVSRAPKPLENSVSAKASTNTSRSVPSPAK
STPNSSVPASAPAPSLTRSQLDRVEALLSPEDKISLNMAKPFRELEPELVTRRKNDFQRLYTNDREDYLGK
LERDITKFFCDRGFLEIKSPILIPAEYVERMGINNDTELSKQIFRVDKNLCLRPMLAPTLYNYLRKLDRILPGP
IKIFEVGPCYRKESDGKEHLEEFTMVNFCQMGSGCTRENLEALIKEFLDYLEIDFEIVGDSCMVYGDTL
DIMHGDLELSSAVVGPVSLDREWGIDKPWIGAGFGLERLLKVMHGFKNIKRASRSESYYNGISTNL
>M.barkeriF/1-419/
Methanosarcina barkeri str. Fusaro
VERSION YP_304395.1 GI:73668380
MDKKPLDVLISATGLWMSRTGTLHKIKHHEVSRSKIYIEMACGDHLVVNNSRSCRTARAFRHHKYRKTC
KRCRVSDEDINNFLTRDTESKNSVKVRVVSAPKVKKAMPKSVSRAPKPLENSVSAKASTNTSRSVPSPAK
STPNSSVPASAPAPSLTRSQLDRVEALLSPEDKISLNMAKPFRELEPELVTRRKNDFQRLYTNDREDYLGKLE
RDITKFFCDRGFLEIKSPILIPAEYVERMGINNDTELSKQIFRVDKNLCLRPMLAPTLYNYLRKLDRILPGPIKI
FEVGPCYRKESDGKEHLEEFTMVNFCQMGSGCTRENLEALIKEFLDYLEIDFEIVGDSCMVYGDTLDI
MHGDLELSSAVVGPVSLDREWGIDKPWIGAGFGLERLLKVMHGFKNIKRASRSESYYNGISTNL
>M.mazei/1-454
Methanosarcina mazei Go1
VERSION NP_633469.1 GI:21227547
MDKKPLDVLISATGLWMSRTGTLHKIKHHEVSRSKIYIEMACGDHLVVNNSRSCRTARAFRHHKYRKTCK
RCRVSDEDLNKFLTKANEDQTSVKVKVVSAPTRTKKAMPKSVARAPKPLENTEAAQAQPSGSKFSPAI
PVSTQESVSVPASVSTSISSISTGATASALVKGNTNPITSMSAPVQASAPALTKSQTDRLEVLLNPKDEISL
NSGKPFRELESELLSRRKKDLQQIYAEERENYLGKLEREITRFFVDRGFLEIKSPILIPLEYIERMGIDNDTELS
KQIFRVDKNFCLRPMLAPNLYNYLRKLDRALPDPIKIFEIGPCYRKESDGKEHLEEFTMLNFCQMGSGC
TRENLESIITDFLNHLGIDFKIVGDSCMVYGDTLDVMHGDLELSSAVVGPIPLDREWGIDKPWIGAGF
GLERLLKVKHDFKNIKRAARSESYYNGISTNL
>M.acetivorans/1-443
Methanosarcina acetivorans C2A
VERSION NP_615128.2 GI:161484944
MDKKPLDTLISATGLWMSRTGMIHKIKHHEVSRSKIYIEMACGERLVVNNSRSSRTARALRHHKYRKTCR
HCRVSDEDINNFLTKTSEEKITVKVKVVSAPRVPKAMPKSVARAPKPLEATAQVPLSGSKPAPATPVSA
PAQAPAPSTGSASATSASAQRMANSAAAPAAPVPTSAPALTKGQLDRLEGLLSPKDEISLDSEKPFRE
LESELLSRRKKDLKRIYAEERENYLGKLEREITKFFVDRGFLEIKSPILIPAEYVERMGINSDTELSKQVFRIDK
NFCLRPMLAPNLYNYLRKLDRALPDPIKIFEIGPCYRKESDGKEHLEEFTMLNFCQMGSGCTRENLEAII
TEFLNHLGIDFEIIGDSCMVYGNTLDVMHDDLELSSAVVGPVPLDREWGIDKPWIGAGFGLERLLKV
MHGFKNIKRAARSESYYNGISTNL
>M.thermophila/1-478
Methanosarcina thermophila,VERSION DQ017250.1 GI:67773308
MDKKPLNTLISATGLWMSRTGKLHKIRHHEVSKRKIYIEMECGERLVVNNSRSCRAARALRHHKYRKIC
KHCRVSDEDLNKFLTRTNEDKSNAKVTVVSAPKIRKVMPKSVARTPKPLENTAPVQTLPSESQPAPTTPIS
ASTTAPASTSTTAPAPASTTAPAPASTTAPASASTTISTSAMPASTSAQGTTKFNYISGGFPRPIPVQASAP
ALTKSQIDRLQGLLSPKDEISLDSGTPFRKLESELLSRRRKDLKQIYAEEREHYLGKLEREITKFFVDRGFLEIK
SPILIPMEYIERMGIDNDKELSKQIFRVDNNFCLRPMLAPNLYNYLRKLNRALPDPIKIFEIGPCYRKESDG
KEHLEEFTMLNFCQMGSGCTRENLEAIIKDFLDYLGIDFEIVGDSCMVYGDTLDVMHGDLELSSAVV
GPVPMDRDGINKPWIGAGFGLERLLKVMHNFKNIKRASRSESYYNGISTNL
>M.burtonii/1-416
Methanococcoides burtoniii DSM 6242, VERSION YP_566710.1 GI:91774018
MEKQLLDVLVELNGVWLSRSGLLHGIRNFEITTKHIHIETDCGARFTVRNSRSSRSARSLRHNKYRKPCKR
CRPADEQIDRFVKKTFKEKRQTVSVFSSPKKHVPKKPKVAVIKSFSISTPSPKEASVSNSIPTPSISVVKDEV
KVPEVKYTPSQIERLKTLMSPDDKIPIQDELPEFKVLEKELIQRRRDDLKKMYEEDREDRLGKLERDITEFFV
DRGFLEIKSPIMIPFEYIERMGIDKDDHLNKQIFRVDESMCLRPMLAPCLYNYLRKLDKVLPDPIRIFEIGP
CYRKESDGSSHLEEFTMVNFCQMGSGCTRENMEALIDEFLEHLGIEYEIEADNCMVYGDTIDIMHGD
LELSSAVVGPIPLDREWGVNKPWMGAGFGLERLLKVRHNYTNIRRASRSELYYNGINTNL
>D.hafniense_DCB-2/1-279
Desulfitobacterium hafniense DCB-2
VERSION YP_002461289.1 GI:219670854
MSSFWTKVQYQRLKELNASGEQLEMGFSDEALSRDRAFQGIEHQLMSQGKRHLEQLRTVKHRPALLEL
EEGLAKALHQQGFVQVVTPTIITKSALAKMITIGEDHPLFSQVFWLDGKKCLRPMLAPNLYTLWRELERL
DRGFLEIKSPIMIPFEYIERMGIDKDDHLNKQIFRVDESMCLRPMLAPCLYNYLRKLDKVLPDPIRIFEIGP
CYRKESDGSSHLEEFTMVNFCQMGSGCTRENMEALIDEFLEHLGIEYEIEADNCMVYGDTIDIMHGD
LELSSAVVGPIPLDREWGVNKPWMGAGFGLERLLKVRHNYTNIRRASRSELYYNGINTNL
>D.hafniense_Y51/1-312
Desulfitobacterium hafniense Y51
VERSION YP_521192.1 GI:89897705
MDRIDHTDSKFVQAGETPVLPATFMFLTRRDPPLSSFWTKVQYQRLKELNASGEQLEMGFSDALSRDR
AFQGIEHQLMSQGKRHLEQLRTVKHRPALLELEEGLAKALHQQGFVQVVTPTIITKSALAKMITIGEDH
PLFSQVFWLDGKKCLRPMLAPNLYTLWRELERLWDKPIRIFEIGTCYRKESQGAQHLNEFTMLNLTELGT
PLEERHQRLEDMARWVLEAAGIREFELVTESSVVYGDTVDVMKGDLELASGAMGPHFLDEKWEIVD
PWVGLGFGLERLLMIREGTQHVQSMARSLSYLDGVRLNIN
>D.hafniensePCP1/1-288
Desulfitobacterium hafniense
VERSION AY692340.1 GI:53771772
MFLTRRDPPLSSFWTKVQYQRLKELNASGEQLEMGFSDALSRDRAFQGIEHQLMSQGKRHLEQLRTV
KHRPALLELEEKLAKALHQQGFVQVVTPTIITKSALAKMTIGEDHPLFSQVFWLDGKKCLRPMLAPNLY
TLWRELERLWDKPIRIFEIGTCYRKESQGAQHLNEFTMLNLTELGTPLEERHQRLEDMARWVLEAAGIRE
FELVTESSVVYGDTVDVMKGDLELASGAMGPHFLDEKWEIFDPWVGLGFGLERLLMIREGTQHVQS
>D.acetoxidans/1-277MARSLSYLDGVRLNIN
Desulfotomaculum acetoxidans DSM 771
VERSION YP_003189614.1 GI:258513392
MSFLWTVSQQKRLSELNASEEEKNMSFSSTSDREAAYKRVEMRLINESKQRLNKLRHETRPAICALENRL
AAALRGAGFVQVATPVILSKKLLGKMTITDEHALFSQVFWIEENKCLRPMLAPNLYYILKDLLRLWEKPV
RIFEIGSCFRKESQGSNHLNEFTMLNLVEWGLPEEQRQKRISELSAKLVMDETGIDEYHLEHAESVVYGET
VDVMHRDIELGSGALGPHFLDGRWGVVGPWVGIGFGLERLLMVEQGGQNVRSMGKSLTYLDG
VRLNI

When the particular tRNA charging (aminoacylation) function has been provided by mutating the tRNA synthetase, then it may not be appropriate to simply use another wild-type tRNA sequence, for example one selected from the above. In this scenario, it will be important to preserve the same tRNA charging (aminoacylation) function. This is accomplished by transferring the mutation(s) in the exemplary tRNA synthetase into an alternate tRNA synthetase backbone, such as one selected from the above.

In this way it should be possible to transfer selected mutations to corresponding tRNA synthetase sequences such as corresponding pytS sequences from other organisms beyond exemplary M. barkeri and/or M. mazei sequences.

Target tRNA synthetase proteins/backbones, may be selected by alignment to known tRNA synthetases such as exemplary M. barkeri and/or M. mazei sequences.

This subject is now illustrated by reference to the pyIS (pyrrolysine tRNA synthetase) sequences but the principles apply equally to the particular tRNA synthetase of interest. For example, FIG. 4 provides an alignment of all PyIS sequences. These can have a low overall % sequence identity. Thus it is important to study the sequence such as by aligning the sequence to known tRNA synthetases (rather than simply to use a low sequence identity score) to ensure that the sequence being used is indeed a tRNA synthetase.

Thus suitably when sequence identity is being considered, suitably it is considered across the tRNA synthetases as in FIG. 4. Suitably the % identity may be as defined from FIG. 4. FIG. 5 shows a diagram of sequence identities between the tRNA synthetases. Suitably the % identity may be as defined from FIG. 5.

It may be useful to focus on the catalytic region. FIG. 6 aligns just the catalytic regions. The aim of this is to provide a tRNA catalytic region from which a high % identity can be defined to capture/identify backbone scaffolds suitable for accepting mutations transplanted in order to produce the same tRNA charging (aminooacylation) function, for example new or unnatural amino acid recognition.

Thus suitably when sequence identity is being considered, suitably it is considered across the catalytic region as in FIG. 6. Suitably the % identity may be as defined from FIG. 6. FIG. 7 shows a diagram of sequence identities between the catalytic regions. Suitably the % identity may be as defined from FIG. 7.

‘Transferring’ or ‘transplanting’ mutations onto an alternate tRNA synthetase backbone can be accomplished by site directed mutogenesis of a nucleotide sequence encoding the tRNA synthetase backbone. This technique is well known in the art. Essentially the backbone pytS sequence is selected (for example using the active site alignment discussed above) and the selected mutations are transferred to (i.e. made in) the corresponding/homologous positions.

When particular amino acid residues are referred to using numeric addresses, unless otherwise apparent, the numbering is taken using MbPyIRS (Methanosarcino barkeri pyrrolysyt-tRNA synthetase) amino acid sequence as the reference sequence (i.e. as encoded by the publicly available wild type Methonosarcina barkeri PyIS gene Accession number Q46E77):

MDKKPLDVLI SATGLWNSRT GTLHKIKHYE VSRSKIYIEM ACGDHLVVNN SRSCRTARAF
RHHKYRKTCK RCRVSDEDIN NFLTRSTEGK TSVKVKVVSA PKVKKAMPKS VSRAPKPLEN
PVSAKASTDT SRSVPSPAKS TPNSPVPTSA PAPSLTRSQL DRVEALLSPE DKISLNIAKP
FRELESELVT RRKNDFQRLY TNDREDYLGK LERDITKFFV DRDFLEIKSP ILIPAEYVER
MGINNDTELS KQIFRVDKNL CLRPMLAPTL YNYLRKLDRI LPDPIKIFEV GPCYRKESDG
KEHLEEFTMV NFCQMGSGCT RENLESLIKE FLDYLEIDFE IVGDSCMVYG DTLDIMHGDL
ELSSAVVGPV PLDREWGIDK PWIGAGFGLE RLLKVMHGFK NIKRASRSES YYNGISTNL

This is to be used as is well understood in the art to locate the residue of interest. This is not always a strict counting exercise—attention must be paid to the context or alignment. For example, if the protein of interest is of a slightly different length, then location of the correct residue in that sequence corresponding to (for example) Y349 may require the sequences to be aligned and the equivalent or corresponding residue picked, rather than simply taking the 349th residue of the sequence of interest. This is well within the ambit of the skilled reader.

Notation for mutations used herein is the standard in the art. For example Y349W means that the amino acid corresponding to Y at position 349 of the wild type sequence is replaced with W.

The transplantation of mutations between alternate tRNA backbones is now Illustrated with reference to exemplary M. bakeri and M. mazei sequences, but the same principles apply equally to transplantation onto or from other backbones.

For example Mb AcKRS is an engineered synthetase for the incorporation of AcK

Parental protein/bockbone: M. barkeri PyIS

Mutations: L266V. L2701. Y271F, L274A, C317F

Mb PCKRS: engineered synthetase for the incorporation of PCK

Parental protein/backbone: M. barkeri PyIS

Mutations: M241F, A267S, Y271C, L274M

Synthetases with the some substrate specificities can be obtained by transplanting these mutations Into M. mazei PyIS. The sequence homology of the two synthetases con be seen in FIG. 8. Thus the following synthetases may be generated by transplantation of the mutations from the Mb backbone onto the Mm tRNA backbone:

Mm AcKRS introducing mutations L301V. L305I, Y306F. L309A, C348F into M. mazei PyIS. and
Mm PCKRS introducing mutations M276F, A3025, Y306C, L309M into M. mazei PyIS.

Full length sequences of these exemplary transplanted rmutation synthetases are given below.

>Mb_PylS/1-419
MDKKPLDVLISATGLWMSRTGTLHKIKHHEVSRSKIYIEMACGDHLVVNNSRSCRTARAFRHHKYRKTC
KRCRVSDEDINNFLTRSTESKNSVKVRVVSAPKVKKAMPKSVSRAPKPLENSCSAKASTNTSRSVPSPAK
STPNSSVPASAPAPSLTRSQLDRVEALLSPEDKISLNMAKPFRELEPELVTRRKNDFQRLYTNDREDYLGK
LERDITKFFVDRGFLEIKSPILIPAEYVERMGINNDTELSKQIFRVDKNLCLRPMLAPTLYNYLRKLDRILPGP
IKIFEVGPCYRKESDGKEHLEEFTMVNFCQMGSGCTRENLEALIKEFLDYLEIDFEIVGDSCMVYGDTL
DIMHGDLELSSAVVGPVSLDREWGIDKPWIGAGFGLERLLKVMHGFKNIKRASRSESYYNGISTNL
>Mb_AcKRS/1-419
MDKKPLDVLISATGLWMSRTGTLHKIKHHEVSRSKIYIEMACGDHLVVNNSRSCRTARAFRHHKYRKTC
KRCRVSDEDINNFLTRSTESKNSVKVRVVSAPKVKKAMPKSVSRAPKPLENSCSAKASTNTSRSVPSPAK
STPNSSVPASAPAPSLTRSQLDRVEALLSPEDKISLNMAKPFRELEPELVTRRKNDFQRLYTNDREDYLGK
LERDITKFFVDRGFLEIKSPILIPAEYVERMGINNDTELSKQIFRVDKNLCLRPMLAPTLYNYLRKLDRILPG
PIKIFEVGPCYRKESDGKEHLEEFTMVNFCQMGSGCTRENLEALIKEFLDYLEIDFEIVGDSCMVYGDTL
DIMHGDLELSSAVVGPVSLDREWGIDKPWIGAGFGLERLLKVMHGFKNIKRASRSESYYNGISTNL
>Mb_PCKRS/1-419
MDKKPLDVLISATGLWMSRTGTLHKIKHHEVSRSKIYIEMACGDHLVVNNSRSCRTARAFRHHKYRKTC
KRCRVSDEDINNFLTRSTESKNSVKVRVVSAPKVKKAMPKSVSRAPKPLENSCSAKASTNTSRSVPSPAK
STPNSSVPASAPAPSLTRSQLDRVEALLSPEDKISLNMAKPFRELEPELVTRRKNDFQRLYTNDREDYLGK
LERDITKFFVDRGFLEIKSPILIPAEYVERMGINNDTELSKQIFRVDKNLCLRPMLAPTLYNYLRKLDRILPGP
IKIFEVGPCYRKESDGKEHLEEFTMVNFCQMGSGCTRENLEALIKEFLDYLEIDFEIVGDSCMVYGDTL
DIMHGDLELSSAVVGPVSLDREWGIDKPWIGAGFGLERLLKVMHGFKNIKRASRSESYYNGISTNL
>Mm_PylS/1-454
MDKKPLDVLISATGLWMSRTGTLHKIKHHEVSRSKIYIEMACGDHLVVNNSRSCRTARAFRHHKYRKTCK
RCRVSDEDINNFLTRSTESKNSVKVRVVSAPKVKKAMPKSVSRAPKPLENSCSAKASTNTSRSVPSPAI
PVSTQESVSVPASVSTSISSISTGATASALVKGNTNPITSMSAPVQASAPALTKSQTDRLEVLLNPKDEISL
NSGKPFRELESELLSRRKKDLQQIYAEERENYLGKLEREITRFFVDRGFLEIKSPILIPLEYIERMGIDNDTELS
KQIFRVDKNFCLRPMLAPNLYNYLRKLDRALPDPIKIFEIGPCYRKESDGKEHLEEFTMLNFCQMGSGC
TRENLESIITDFLNHLGIDFKIVGDSCMVYGDTLDVMHGDLELSSAVVGPIPLDREWGIDKPWIGAGF
GLERLLKVKHDFKNIKRAARSESYYNGISTNL
>Mm_AcKRS/1-454
MDKKPLDVLISATGLWMSRTGTLHKIKHHEVSRSKIYIEMACGDHLVVNNSRSCRTARAFRHHKYRKTCK
RCRVSDEDINNFLTRSTESKNSVKVRVVSAPKVKKAMPKSVSRAPKPLENSCSAKASTNTSRSVPSPAI
PVSTQESVSVPASVSTSISSISTGATASALVKGNTNPITSMSAPVQASAPALTKSQTDRLEVLLNPKDEISL
NSGKPFRELESELLSRRKKDLQQIYAEERENYLGKLEREITRFFVDRGFLEIKSPILIPLEYIERMGIDNDTELS
KQIFRVDKNFCLRPMLAPNLYNYLRKLDRALPDPIKIFEIGPCYRKESDGKEHLEEFTMLNFCQMGSGC
TRENLESIITDFLNHLGIDFKIVGDSCMVYGDTLDVMHGDLELSSAVVGPIPLDREWGIDKPWIGAGF
GLERLLKVKHDFKNIKRAARSESYYNGISTNL
>Mm_PCKRS/1-454
MDKKPLDVLISATGLWMSRTGTLHKIKHHEVSRSKIYIEMACGDHLVVNNSRSCRTARAFRHHKYRKTCK
RCRVSDEDINNFLTRSTESKNSVKVRVVSAPKVKKAMPKSVSRAPKPLENSCSAKASTNTSRSVPSPAI
PVSTQESVSVPASVSTSISSISTGATASALVKGNTNPITSMSAPVQASAPALTKSQTDRLEVLLNPKDEISL
NSGKPFRELESELLSRRKKDLQQIYAEERENYLGKLEREITRFFVDRGFLEIKSPILIPLEYIERMGIDNDTELSK
QIFRVDKNFCLRPMLAPNLYNYLRKLDRALPDPIKIFEIGPCYRKESDGKEHLEEFTMLNFCQMGSGC
TRENLESIITDFLNHLGIDFKIVGDSCMVYGDTLDVMHGDLELSSAVVGPIPLDREWGIDKPWIGAGF
GLERLLKVKHDFKNIKRAARSESYYNGISTNL

The same principle applies equally to other mutations and/or to other backbones.

Transplanted polypeptides produced in this manner should advantageously be tested to ensure that the desired function/substrate specificities have been preserved.

In the method according to the invention, said genetic incorporation preferably uses an orthogonal or expanded genetic code, in which one or more specific orthogonal codons have been allocated to encode the specific lysine residue with the lysine side group chain protected so that it can be genetically incorporated by using an orthogonal tRNA synthetase/tRNA pair. The orthogonal tRNA synthetase/tRNA pair con in principle be any such pair capable of charging the tRNA with the protected lysine and capable of incorporating that protected lysine into the polypeptide chain in response to the orthogonal codon.

The orthogonal codon may be the orthogonal codon amber, ochre, opal or a quadruplet codon. The codon simply has to correspond to the orthogonal tRNA which will be used to carry the protected lysine molecule. Preferably the orthogonal codon is amber.

It should be noted that the specific examples shown herein have used the amber codon and the corresponding tRNA/tRNA synthetase. As noted above, these may be varied. Alternatively, in order to use other codons without going to the trouble of using or selecting alternative tRNA/tRNA synthetase pairs capable of working with the protected lysine, the anticodon region of the tRNA may simply be swapped for the desired anticodon region for the codon of choice. The anticodon region is not involved in the charging or incorporation functions of the tRNA nor recognition by the tRNA synthetase so such swaps are entirely within the ambit of the skilled operator.

Thus alternative orthogonal tRNA synthetase/tRNA pairs may be used it desired.

Preferably the orthogonal synthetase/tRNA par are Methanosorcina barkeri MS pyrrolysine tRNA synthetase (MbPyIRS) Y349W and its cognate amber suppressor tRNA (MbtRNA_{CUA}).

The polypeptides of the invention are made by translation of an RNA comprising the orthogonal codon (such as the amber codon) at the position at which it is desired to incorporate the unnatural amino acid (such as delta substituted lysine). This RNA is typically made by transcription of a nucleic acid such as DNA encoding the polypeptide. This transcription is typically carried out in a host cel in which the polypeptide is being made. The introduction of the orthogonal codon into the desired site in the nucleic acid is well within the ambit of the person skiled in the art. This nucleic acid such as DNA may be made by any suitable means such as recombinant manipulation and ligation. PCR, site-directed mutagenesis or chemical synthesis or any other suitable technique.

### INDUSTRIAL APPLICABILITY

The ability to efficiently genetically encode unnatural amino acids for native isopeptide bond formation will greatly expand the scope and accessibility of methods for protein ubiquitination, SUMOylation and Neddylation and accelerate research into the effects of these important post-translational modifications.

### DESCRIPTION OF THE DRAWINGS

FIG. 1 shows (A) 1 pyrrolysine, 2 Nε-(t-butyloxycarbonyl)-L-lysine. 3 photocleavable auxiliary-bearing amino acid allowing native chemical ligation (NCL) with ubiquitin 1-75 thioester, 4 Nε-protected γ-thiol-L-lysine (R₃=carbobenzyloxy or 3,4-dimethoxy-o-nitro carbobenzyloxy). 5 δ-thiol-Nε-allyloxycarbonyl)-L-lysine, 6 thiozolidine protected δ-thiol-L-lysine. 7 δ-thiol lysine. 8 δ-hydroxylysine, 9 δ-hydroxy-Nε-(t-butyloxycarbony)-L-lysine, 10 δ-thiol-Nε-(t-butyloxycorbonyl)-L-lysine, 11 δ-methyldisuffanyi-Nc-(t-butyloxycarbonyl)-L-lysine, 12 Nε-(p-nitro carbobenzyloxy)lysine, 13 δ-hydroxy-Nε-(p-nitro carbobenzyloxy)lysine, 14 δ-thiol-Nε-(p-nitro carbobenzyloxy)lysine. (B) Geneticaly directing traceless ubiquitination.

FIG. 2 shows. Incorporation of 7 into recombinant proteins. (A) SDS-PAGE reveals amino acid dependent incorporation of 14 into position 6 of ubiquitin by nitroCbzKRS*/tRNACUA. (B) Blue is deconvoluted mass spectrum of ubiquitin containing pyruvate-derived thiazolidine adducts. Thiazolidine adduct expected mass=9490 Da; found=9490 Da: decarboxylated thiazolidine adduct expected mass=9446 Da: Unmodified expected mass=9420 Da; found=9420 Da. Green spectra is protein treated with 200 mM methoxyamine for 24 h. Expected mass=9420 Da; found=9420 Da.

FIG. 3 shows Geneticaly encoded 7 directs site-specific fraceless isopeptide bond formation via native chemical ligation and desulfurization. (A) SDS-PAGE analysis of ligation. UbSR is ubiquitin thioester. UbSHK6 is Ubiquitin His6 with 7 at position 6. Ub₂SH is the ligation product. (B) Deconvoluted MS spectrum and SDS PAGE of K6 linked diubiquitin resulting from desulfurization DiUbSHK6-His₆ and purification. Full spectra are presented in Supplementary FIG. 9.

FIG. 4 shows alignment of PyIS sequences.

FIG. 5 shows sequence identity of PyIS sequences.

FIG. 6 shows alignment of the catalytic domain of PyIS sequences (from 350 to 480; numbering from alignment of FIG. 4).

FIG. 7 shows sequence identity of the catalytic domains of PyIS sequences.

FIG. 8 shows alignment of synthetases with transplanted mutations based on M. barkeri PyIS or M. mazei PyIS. The red asterisks indicate the mutated positions.

FIG. 9 shows a diagram of a method.

FIG. 10 shows (A) Crystal structure of pyrrolysine (grey) bound to M. mazei PyIRS. N311 and Y349 (green) are within 5 Å of the δ-carbon (sphere) of pyrrolysine. (B) Residues that were randomized to allow selection for nitroCbzKRS are in green and cyon. Residues in cyan are those found mutated in the selected synthetase (Y27M. L274G and C313A). Figures created using Pymol (www.pymol.org) and PDB ID 2Q7H.

FIG. 11 shows SDS-PAGE analysis of nickel-offinity purified expression of UbTAG6-His6 in the presence of the δSHKRS1/tRNACUA pair and unnatural amino acids 2 (2 mM), 9 (5 mM) and 10 (5 mM). The δSHKRS1/tRNACUA pair directs the incorporation of each of the unnatural amino acids. The loading in lane 1 has been reduced—10 fold with respect to the other lanes. The last lane shows there is negligible expression of full-length protein in the absence of added unnatural amino acid, indicating that the evolved synthetase does not efficiently use natural amino acids.

FIG. 12 shows (A) ESI-MS characterization of Ni-affinity purified C-terminally His-togged ubiquitin containing hydroxy amino acid 9 at position 6. Expected mass=9503.7 Da; found=9504 Da. Sample was prepared for MS using a C4 Ziptip (Millipore) by aspirating sample after Ni-NIA affinity purification. (B) ESI-MS (Agilent) characterization of Ni-affinity purified C-terminally His-togged ubiquitin containing thiolamino ocid 10 at position 6. Expected mass=9519.8 Da; found=9519.5 Da. (C) Ubiquitin containing 10 at position 6 has been deprotected by treatment with 60% trifluoroacetic acid for 1 h. Expected mass=9419.7 Da: found=9420 Da.

FIG. 13 shows (A) SDS-PAGE analysis of nickel-affinity purified expression of UbTAG6-His6. Lanes 1 and 2 are from cells containing the wild type PyIRS/tRNACUA pair with and without 1 mM 2. Lanes 3 and 4 are from cells containing the nitroCbzKRS/tRNACUA pair with and without 1 mM 12. Lanes 5 and 6 are from cels containing nitroCbzKRS*/tRNACUA with and without 1 mM 8. These data demonstrate that evolved nitroCbzKRS incorporates 12 with an efficiency comparable to PyIRS incorporation of 2. These data also show that the δ-substituted amino acid 14 is not incorporated by the nitroCbzKRS/tRNACUA pair. (B) ESI-MS (Agilent) analysis of ubiquitin containing 12 at position 6. The spectra reveals that the identity of the amino acid present at position 6 of ubiquitin after purification is in fact lysine and the p-nitrocarbobenzyloxy group has been removed in situ. Expected moss=9387.7 Da; found=9388 Da.

FIG. 14 shows ESI-MS (Agilent) analysis of ubiquitin incorporating 13 at position 6. The spectra demonstrates that in the purified protein the p-nitrocarbobenzyloxy group has been removed in situ, thus allowing the facile incorporation of 8. Expected mass=9403.7 Da found=9403 Da.

FIG. 15 shows Proposed mechanism for the observed in situ removal of p-nitrocarbobenzyloxy from genetically incorporated amino acids. The p-nitro group is reduced to an amine by cellular factors. The p-amino species then undergoes a 1,6-elimination generating incorporated amino acid 7. This forms a thiazoldine adduct with cellular pyruvate which is stable and present in the purified protein. The thiazolidine con be readily ring-opened by mild treatment with 200 mM methoxyamine at neutral pH for 24 h.

FIG. 16 shows LC-MS spectra demonstrating the incorporation of 14 at other lysine sites within ubiquitin and subsequent thiazolidine-ring opening (deprotection). 5-10 mg mL-1 of protein were obtained at each site. (A) K11, (B) K33, (C), K48. Similar data were obtained for the K27, K29 and K63 sites. Expected mass of ubiquitin containing pyruvate thiazolidine adduct=9490 Da; expected moss of ubiquitin containing decarboxylated pyruvate adduct=9446 Da; expected mass of ubiquitin containing deprotected amino acid (i.e. native chemical ligation competent 1,2-amino thiol)=9420 Do.

FIG. 17 shows (A) SDS-PAGE analysis to determine fractions containing DIUb6SHK6-His6 after ion exchange purification. (B) Control ligation carried out with wild type ubiquitin not containing an unnatural amino acid. Conditions were 200 mM Na2HPO4 pH 7.5, 6 M GdnCl, 100 mM MESNa. The data shows that background aminolysis of UbSR is negligible.

FIG. 18 shows Non-deconvoluted and deconvoluted ESI-MS (Agilent) spectra for purified ligation product (DiUbδSHK6-HIs6; expected=17966.6 Da; found=17966.9 Da) and desulfurized product (DiUbK6-His6; expected=17934.5 Da; found=17935.0 Da).

FIG. 19 shows K6-linked diubiquitin was incubated with the indicated deubiquitinase (DUB). Mono- and diubiquitin were resolved by SDS-PAGE and imaged by silver staining.* His-tag has been removed with UCH-L3.

The invention is now described by way of example. These examples are intended to be illustrative, and are not intended to limit the appended claims.

FIG. 20 to 44 show NMR spectra.

### EXAMPLES

### Example 1

We first synthesized Ne Boc protected versions of amino acids bearing 8 substituents (9-11) (Supplementary Schemes 1 & 2 & Experimental). We demonstrated that none of these amino acids are incorporated into proteins in response to the amber codon using the wild-type PyIRS/tRNACUA pair.

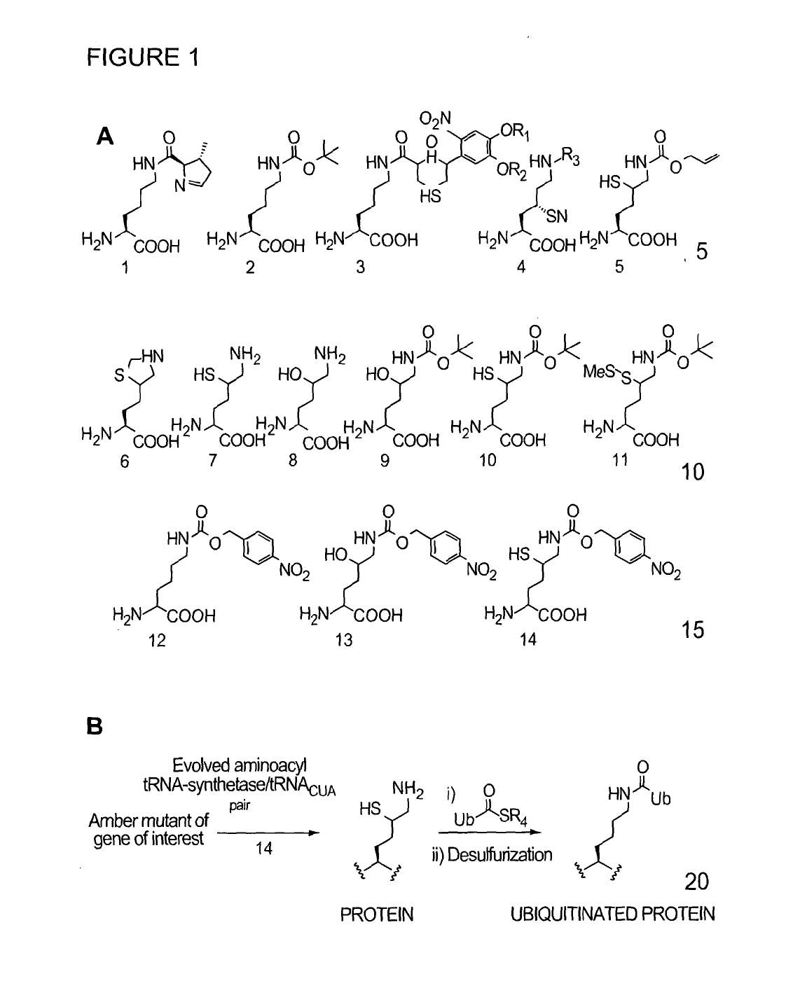

Next, we aimed to discover on evolved PyIRS/tRNACUA pair for the incorporation of amino acids 9-11. Examination of the crystal structure of PyRS in complex with pyrrolysine 20 revealed two prominent residues (N311 and Y349) in the enzyme that are within 5 Å of the 6 carbon of its amino acid substrate (Supplementary FIG. 1). N311 in PyIRS binds to the carbonyl group in the bound pyrrolysine, and mutation of this amino acid destroys the ability of the enzyme to discriminate this substrate from natural amino acids (data not shown). Since this carbonyl group is conserved in our designed substrates we decided to maintain N311 as a potential positive specificity determinant for binding the new unnatural amino acids.

We created a library in which Y349 of MbPyIRS is mutated to all natural amino acids and selected MbPyIRS/tRNACUA variants that confer chloramphenicol resistance on cells bearing a chloramphenicol ocetyl-transferose gene with an amber codon at a permissive site (D112TAG) in the presence of δ-hydroxy-Nε-(t-butyloxycorbony)-L-lysine (9). We performed the initial selections in the presence of 9 since it is valence isoelectronic with its thiol analog (10) but can be prepared in gram quantities in a single step from commercial starting materials (Supplementary Scheme 1), and since we were concerned that a fraction of the δ thiol compound might undergo oxidation that could potentially lead to the selection of synthetases that recognized oxidized forms of the amino acid. These selections yielded a single mutant Y349W, and subsequent selections using the 6 thiol compound (10) directly yielded the same mutation. Selections using a number of more complex libraries did not yield alternative or improved mutants, nor did any of the libraries tested allow the incorporation of the disulfide-protected compound (11, data not shown).

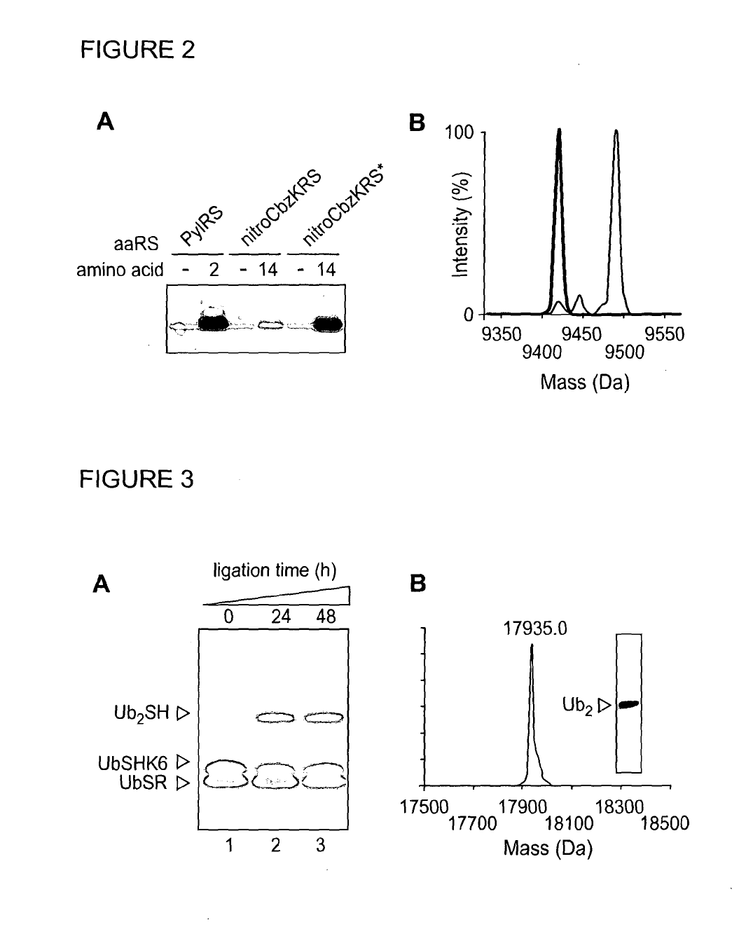

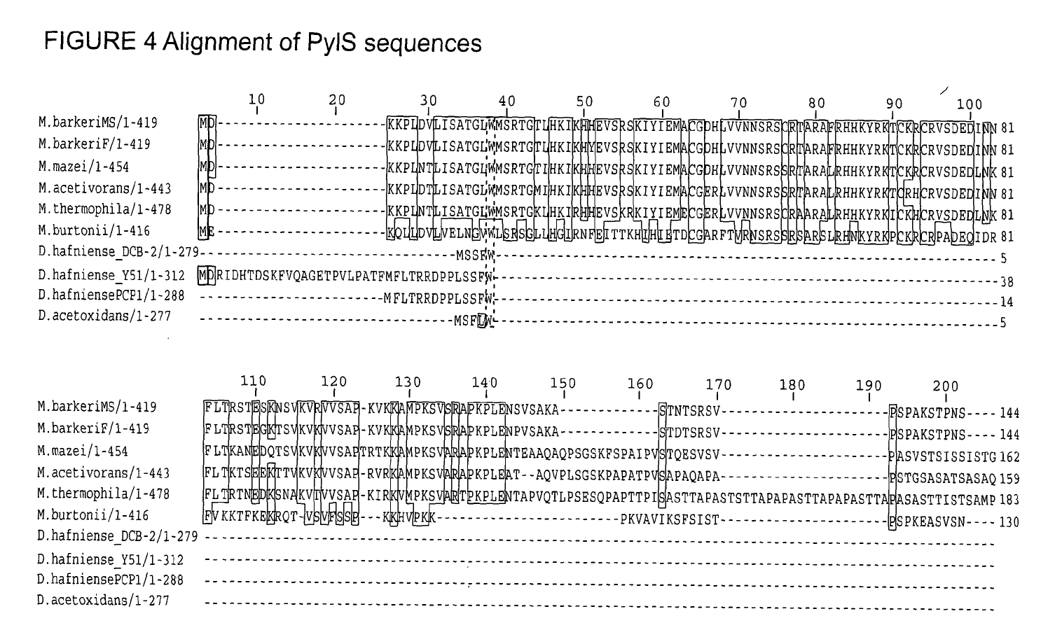

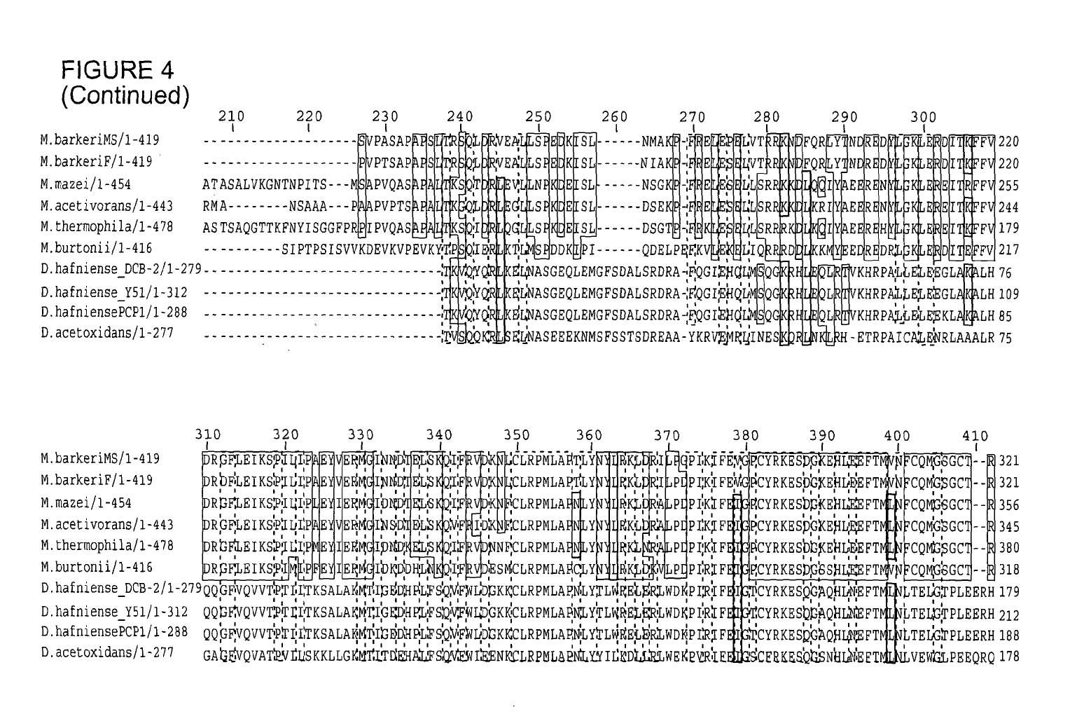

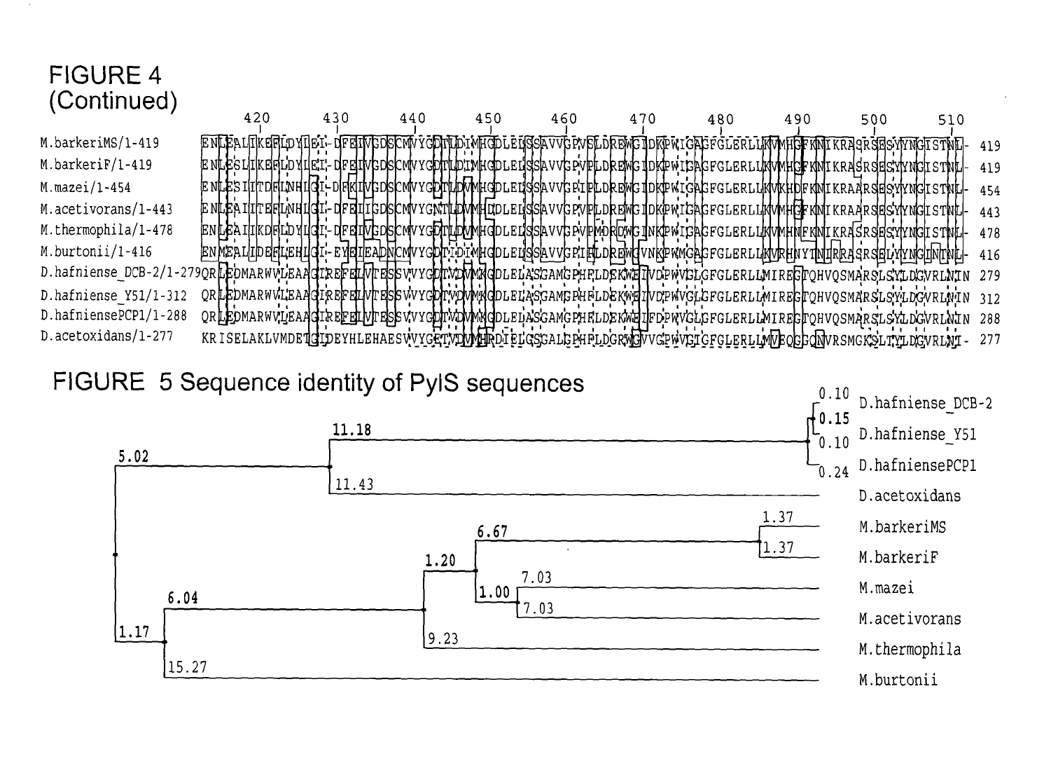

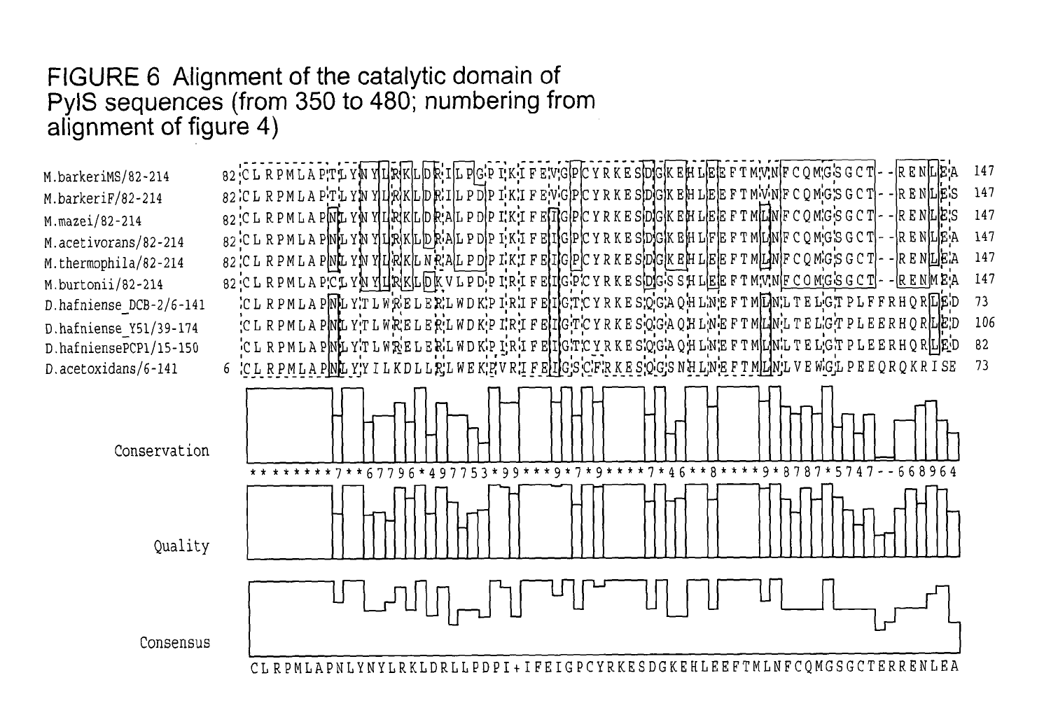

The selected synthetase (dSHKRS)/tRNACUA pair conferred chloramphenicol resistance on cells containing a chloramphenicol acetyltransferase gene with an amber codon at position 112 of 200 μg mL-1 in the presence of 10 and less than 50 μg mL-1 in the absence of 10. We produced C-terminally His-tagged ubiquitin with an amber codon at position 6 from UbTAG6-His6 in the presence of dSHKRS/tRNACUA and 9 or 10, in reasonable yield (0.5 mg L-1 (Supplementary FIG. 2)). No protein was produced in the absence of the unnatural amino acid. The incorporation of each amino acid was conclusively demonstrated by ESI-MS analysis (Supplementary FIG. 3) and ubiquitin bearing the d thiol lysine (7) at position 6, was prepared by the quantitative removal of the Boc group from ubiquitin bearing 10 at position 6 by the addition of 60% TFA for 1 h at 22° C., and characterized by mass spectrometry (Supplementary FIG. 3). Taken together the phenotypic experiments, protein expression experiments and mass spectrometry data conclusively demonstrate that dSHKRS/tRNACUA directs the incorporation of 9 or 10 into recombinant proteins in response to the amber codon and allows the preparation of proteins containing a site specifically incorporated d thiol lysine. However, we were interested in improving two aspects of this approach. First of all the yield of recombinant protein produced when using this synthetase was 10-20 times lower than that obtained with 2 and the PyIRS/tRNACUA pair, a combination that we and others have shown is very efficient 10,18,19. Second the deprotection conditions are denaturing, making this approach to instaling 7 incompatible with proteins that cannot be reversibly refolded. To improve the method we combined our progress up to this point with some observations we hod mode while investigating the scope and evolvobility of the PyIRS/tRNACUA pair. This allowed us to very efficiently install δ substituted derivatives of lysine (7,8) into proteins under native conditions. In the process of investigating the scope of amino acids that can be incorporated using PyIRS/tRNACUA pair we discovered a variant synthetase (nitroCbzKRS) that incorporates Ns-(p-nitro corbobenzyloxy)-L-lysine (12). This synthetase was selected by rounds of positive and negative selection21 on a 109 member synthetase library, which contains all combinations of mutations at M241, A267, Y271, L274 and C313, and the evolved synthetase contain the mutations Y271M, L274G and C313A. The selected nitroCbzKRS/tRNACUA pair conferred chloromphenical resistance on cells containing a chloramphenicol acetyltransferase gene with on amber codon at position 112 of greater than 300 μg mL-1 in the presence of Nε-(p-nitrocarbobenzyloxy)-L-lysine (12) and less than 50 μg mL-1 in the absence of 12. Cells containing the nitroCbzKRS/tRNACUA pair directed expression of UbTAG6-His6 in the presence Ns-(p-nitro carbobenzyloxy)-L-lysine to produce good yields of ubiquitin (10 mg L-1). Ubiquitin expression was clearly amino acid dependent (Supplementary FIG. 4).

The amino acid dependence observed in the phenotypic and protein expression experiments demonstrated that 12 is incorporated during protein translation, and that there is little translational incorporation of natural amino acids in response to the amber codon. However mass spectrometry of the purified protein revealed that the protein contained lysine in place of the unnatural amino acid that was added to celts (Supplementory FIG. 4). We therefore postulated that 12 was incorporated into the protein during cellular translation, but the p-nitro carbobenzyloxy group was subsequently removed from the c amino group of lysine.

We next synthesized delta substituted derivatives of Nε-(p-nitro carbobenzyoxy)-L-lysine (13, 14, Supplementary Scheme 3 and Supplementary Methods), and aimed to site specifically incorporate these into proteins. The incorporation of these amino acids and subsequent removal of p-nitro carbobenzyloxy group, as described above, should lead to a clear mass shift in the protein, corresponding to the moss added by the δ substituent. In contrast to the above case where the mass spectra, but not the amino acid dependent protein expression data, are formally compatible with the direct translational incorporation of lysine this experiment would unambiguously demonstrate that the unnatural amino acid is incorporated in the protein. Moreover, it would provide a direct route to the incorporation of δ substituted lysine derivatives.

As expected the nitroCbzKRS/tRNACUA pair did not direct the efficient incorporation of the delta substituted amino acids (FIG. 2). We therefore combined the mutation in dSHKRS that allows the incorporation of 9 & 10 with those in nitroCbzLysRS for introducing 12 into a new synthetase nitroCbzLysRS*, with the goal of discovering a synthetase that uses 13 and 14 to deliver 8 & 7 into recombinant proteins.

Mass spectrometry of ubiquitin purified from cells in which the nitroCbzKRS*/tRNACUA pair was used to incorporate 13 into ubiquitin in response to an amber codon at position 6 demonstrates the incorporation of 8 (Supplementary FIG. 5). This results from the translational incorporation of 13 into ubiquitin and the subsequent removal of the p-nitro carbobenzyloxy group from the protein.

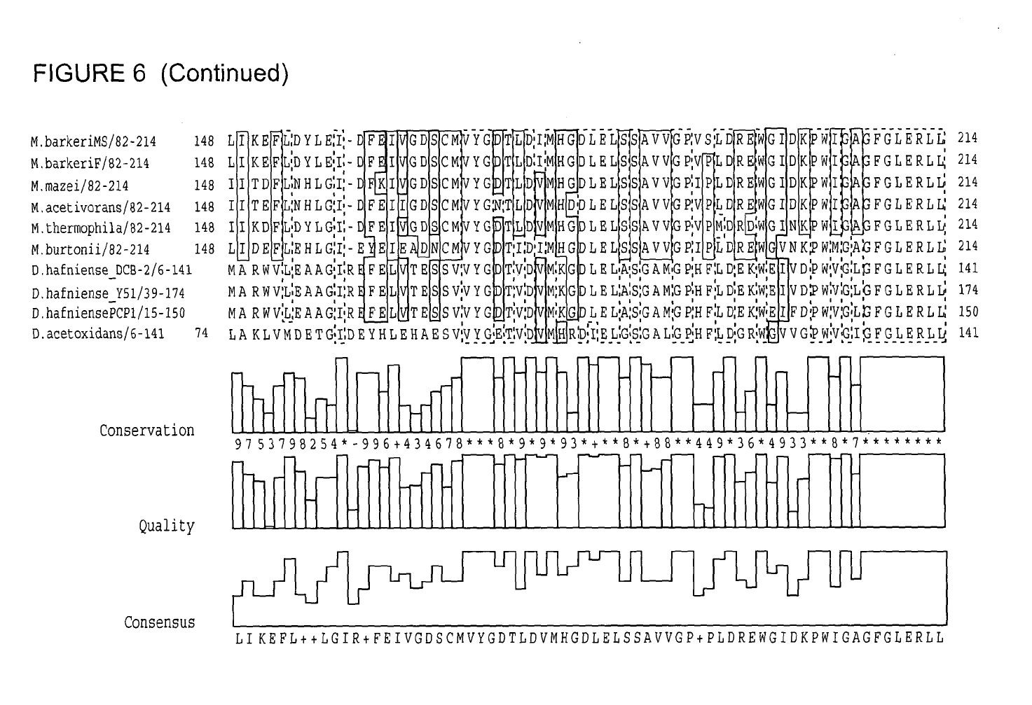

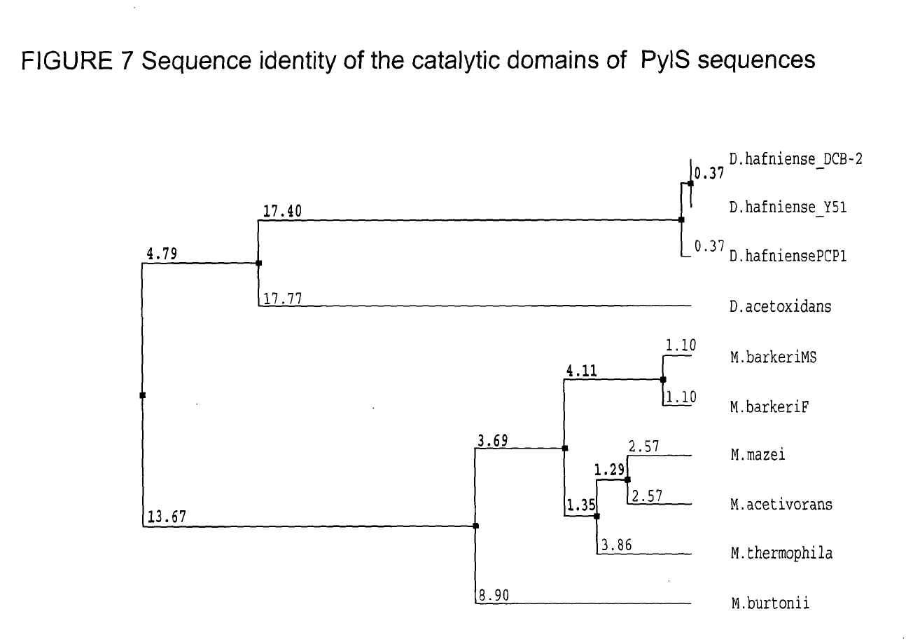

Finally we incorporated d-thiol-Nε-(p-nitro carbobenzyoxy)-L-lysine (14) into ubiquitin at position 6 using the nitroCbzLysRS*/tRNACUA pair. Again, protein expression was amino acid dependent, suggesting that the amino acid was incorporated into the protein during translation (FIG. 2). The yield of recombinant protein (10 mg L-1) was comparable to that for the incorporation of 2 with the PylRS/tRNACUA pair 10. Mass spectrometry revealed a mass of 9490 Da, corresponding to removal of the p-nitro carbobenzyloxy group from the ε amine of tysine and the formation of a thiazolidine adduct between the resulting 1,2 amino thiol and pyruvate 22 (FIG. 2 & Supplementary FIG. 6). A second minor peak corresponds to the decarboxylation of the thiazolidine adduct. These adducts are well precedented for free 1,2 amino thiols resulting from N-terminal cysteines. Treatment of the protein with 200 mM methoxyamine 22 in PBS at pH 7 led to quantitative removal of the pyruvate adducts to reveal d-thiol lysine (7) at the genetically directed site in the protein, as characterized by mass spectrometry. We have repeated this experiment with several sites within ubiquitin and within other proteins, to demonstrate that the steps we have described for K6 within ubiquitin have general utility (Supplementary FIG. 7). While we do not yet know the exact mechanism by which the nitro substituted Cbz groups are removed from the protein a likely mechanism would include the reduction of the aromatic nitro group to an aniline by cellular factors released upon lysis and the subsequent fragmentation of the aniline to reveal a free epsilon amino group(Supplementary FIG. 6).

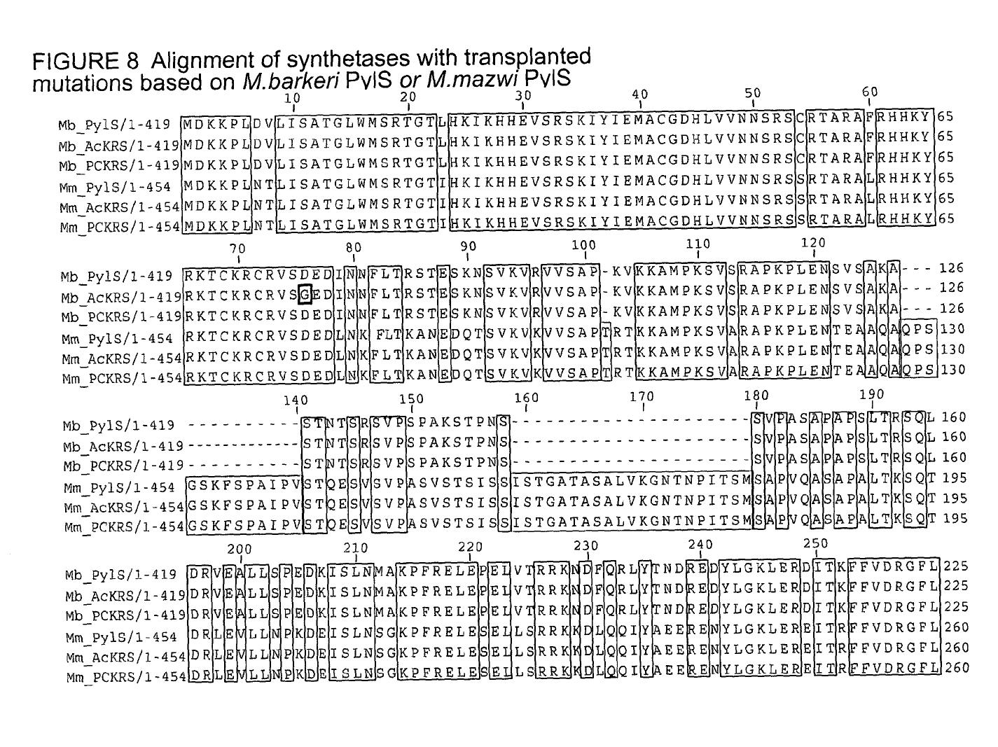

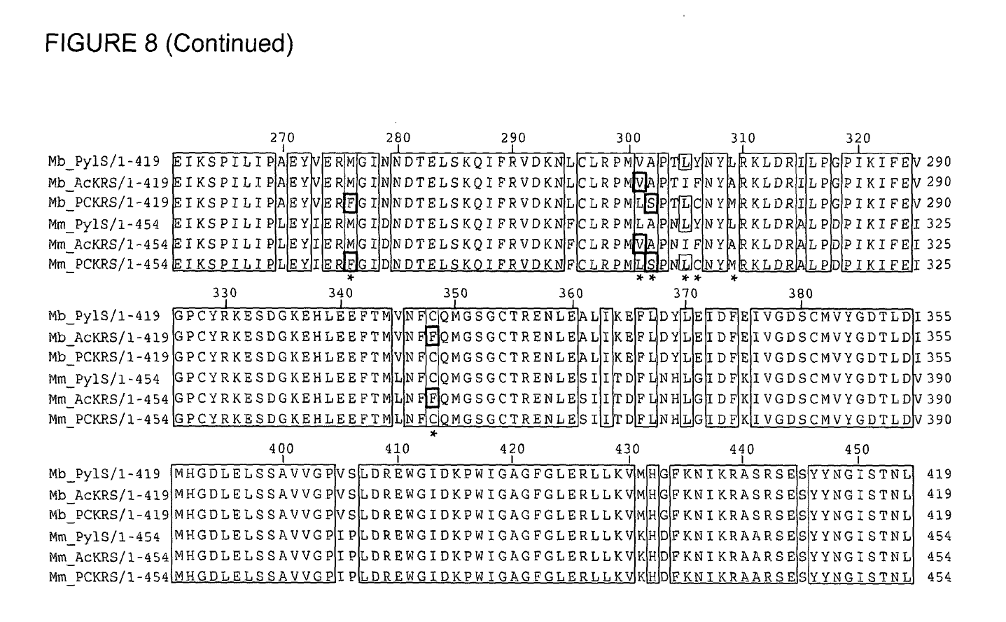

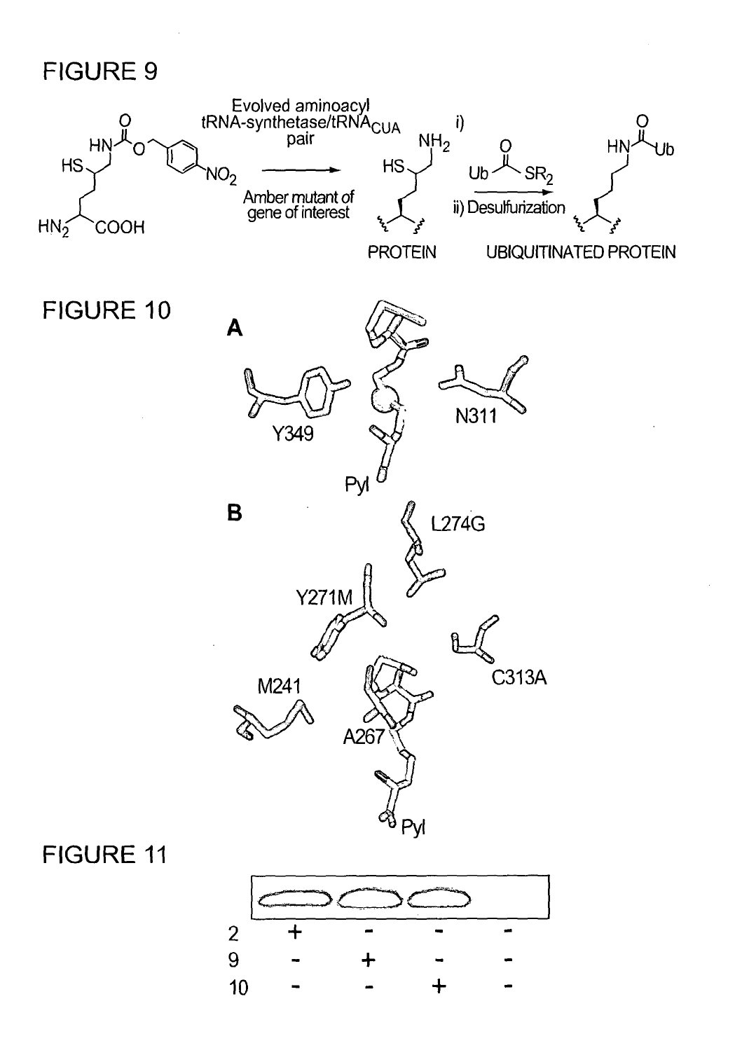

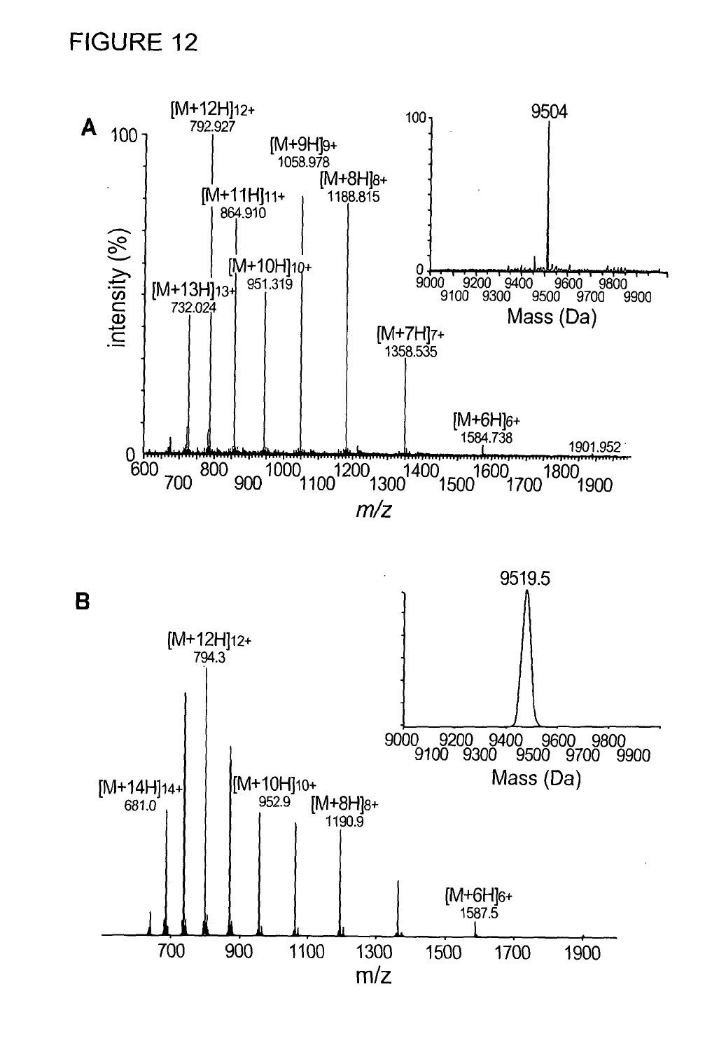

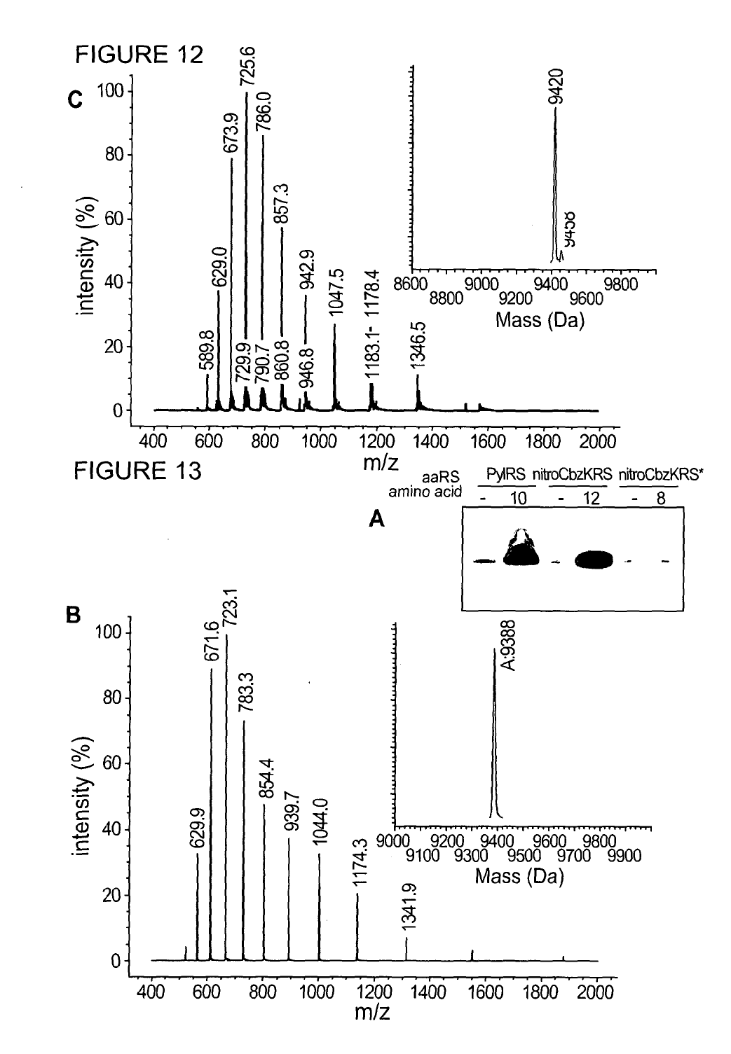

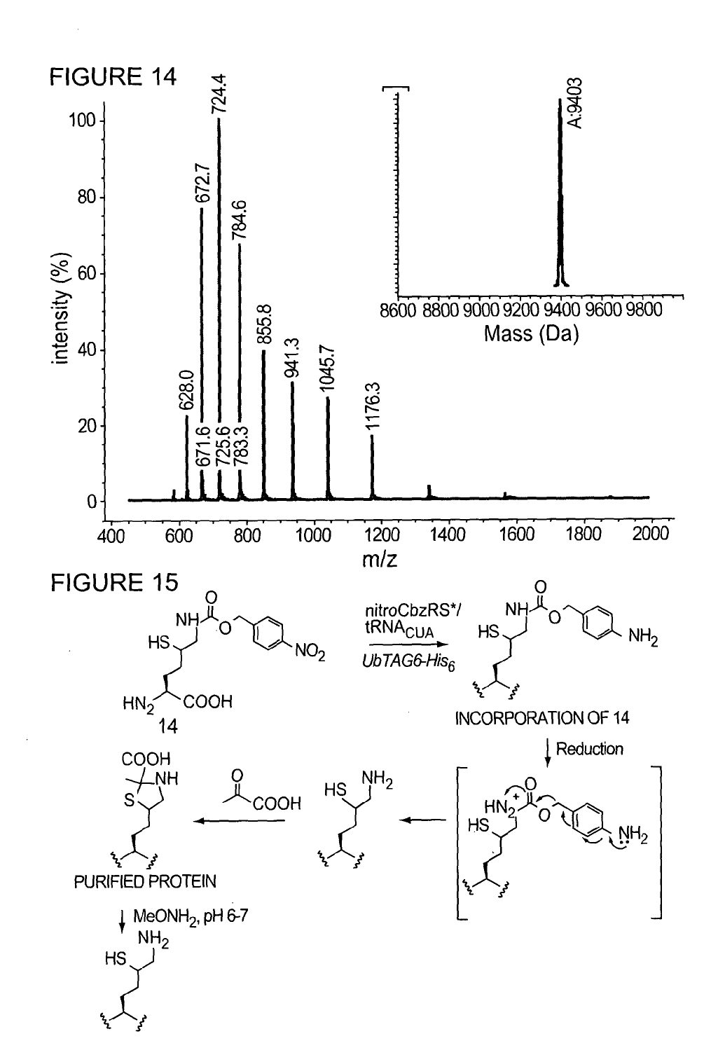

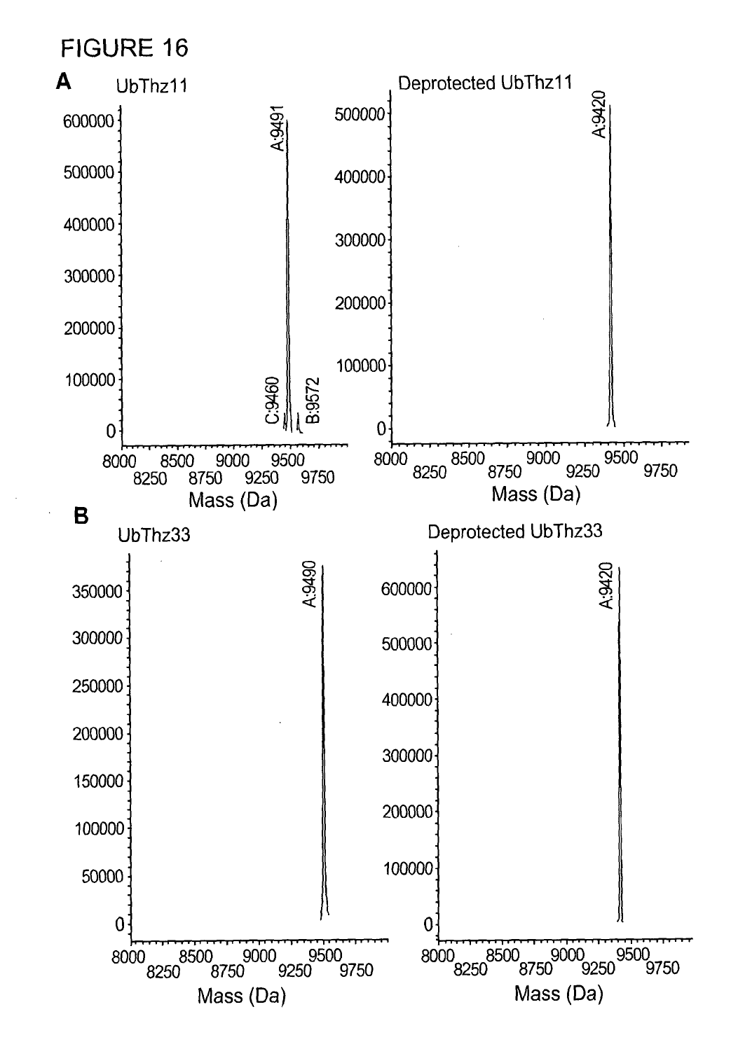

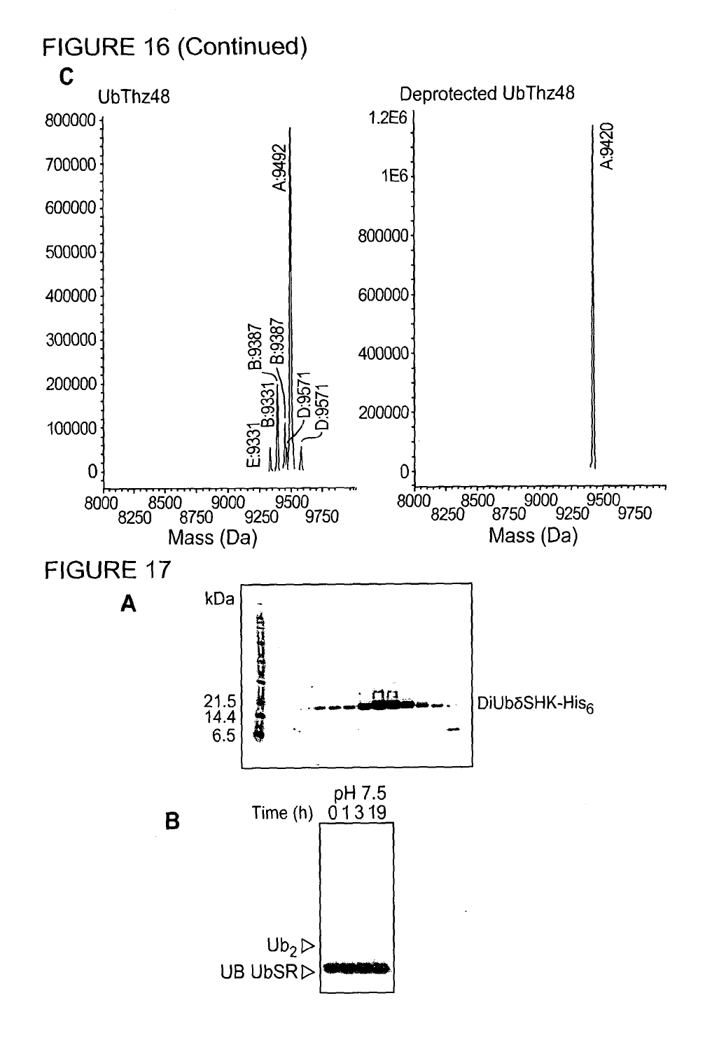

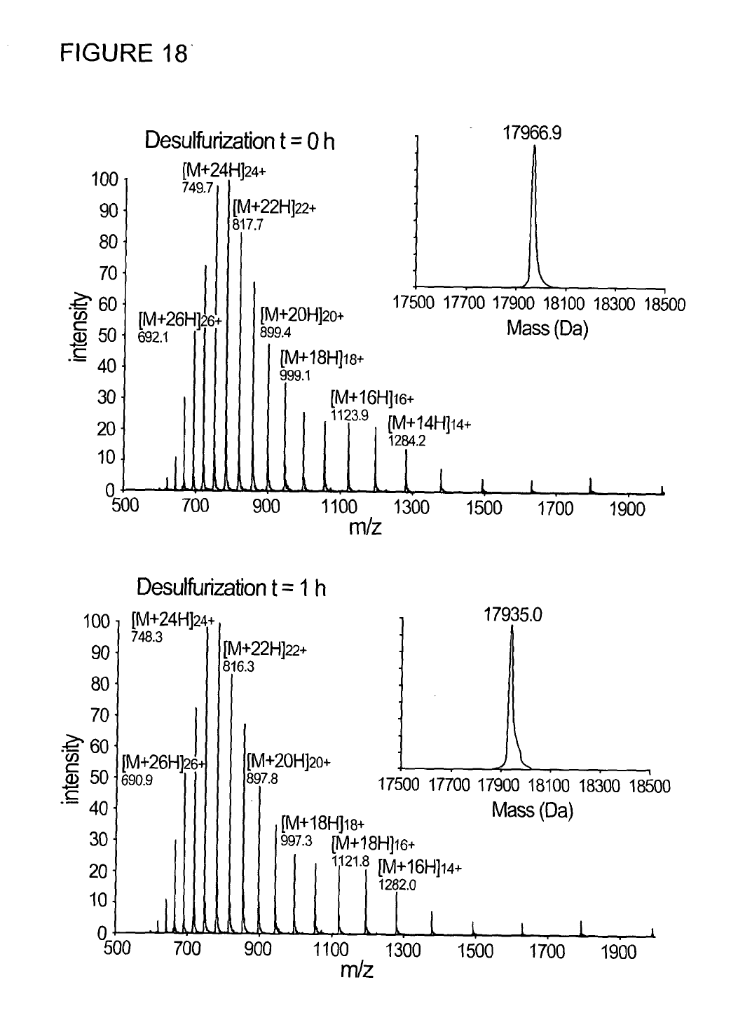

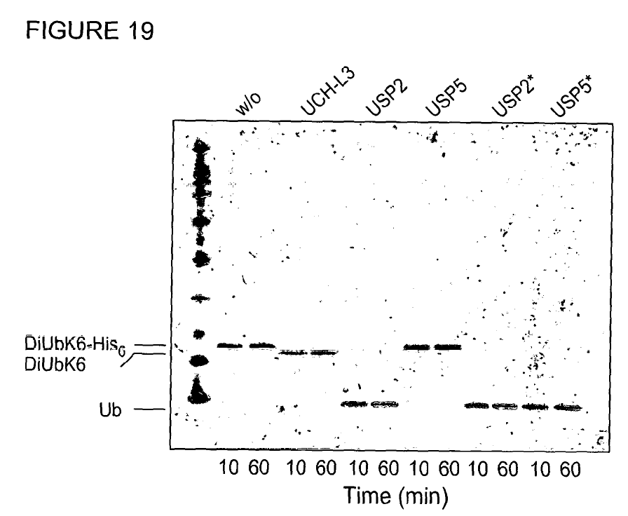

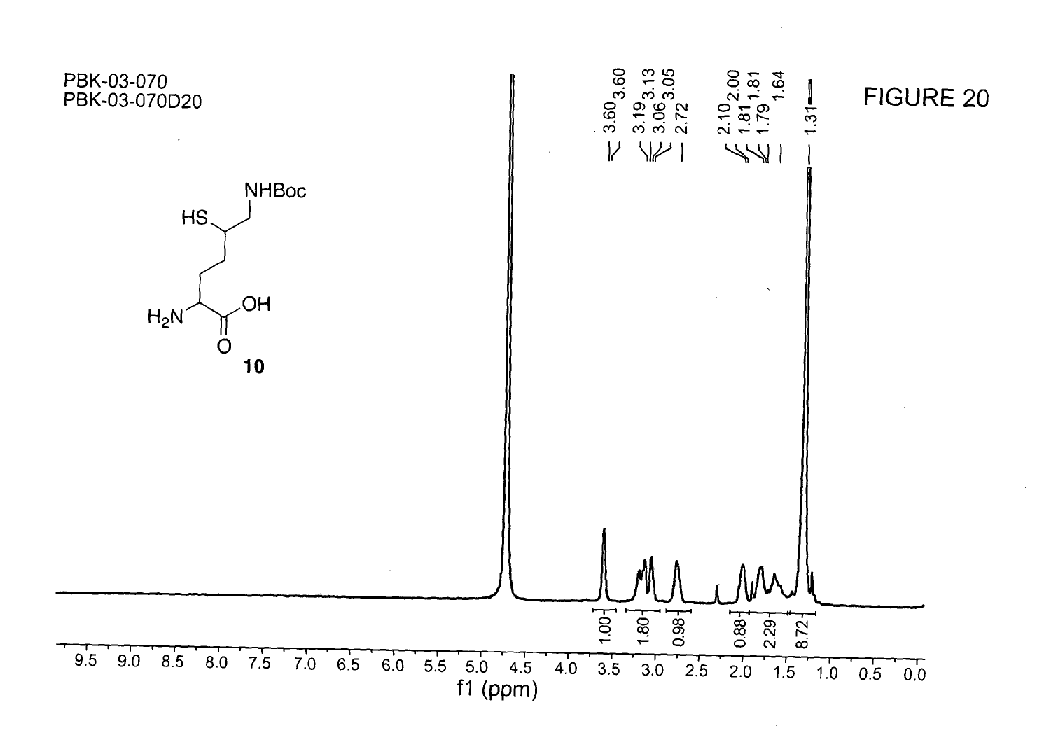

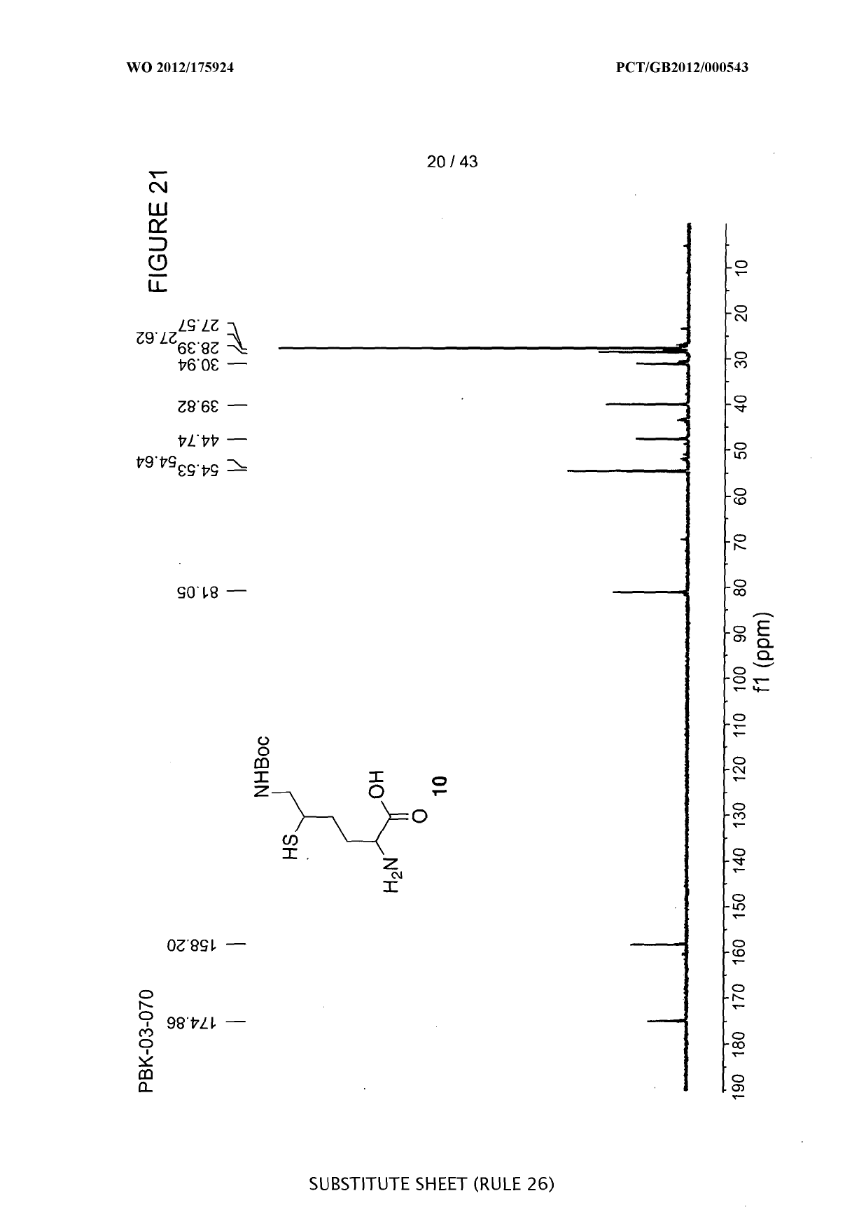

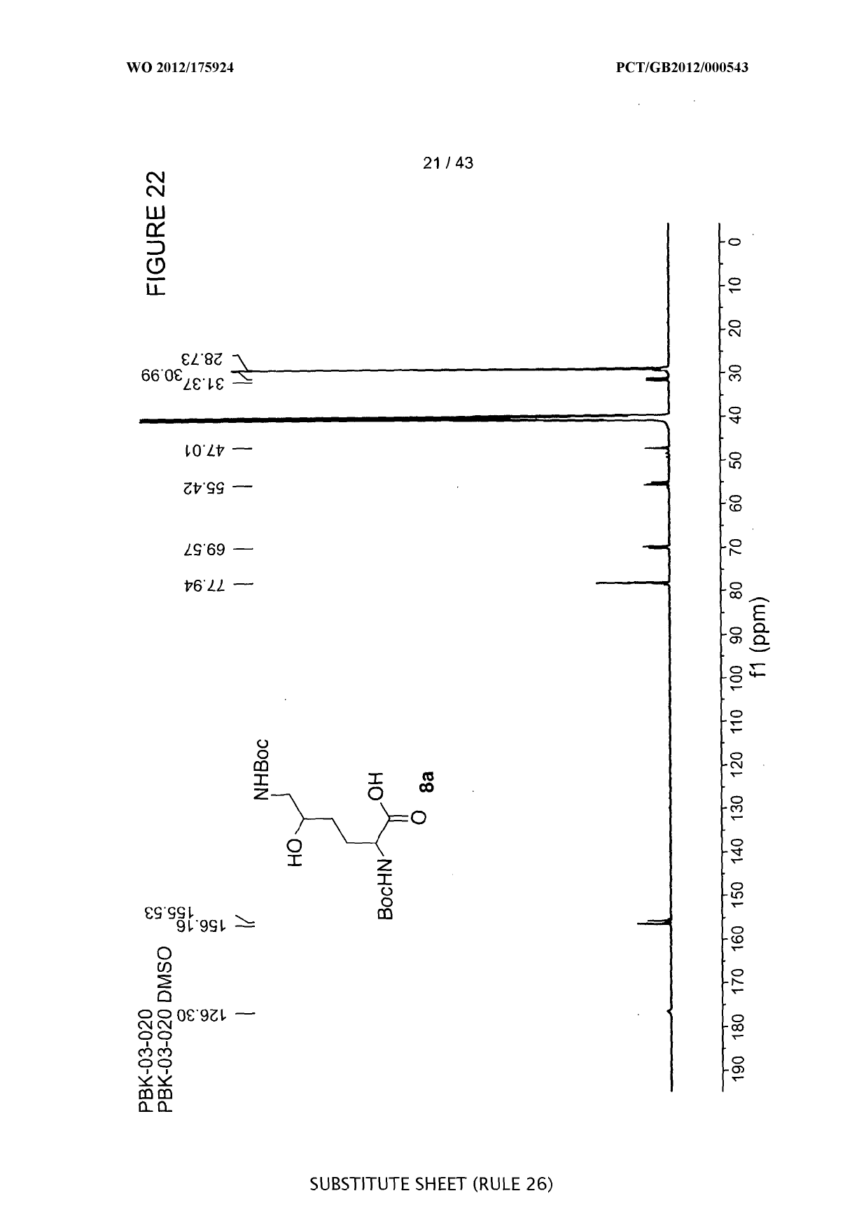

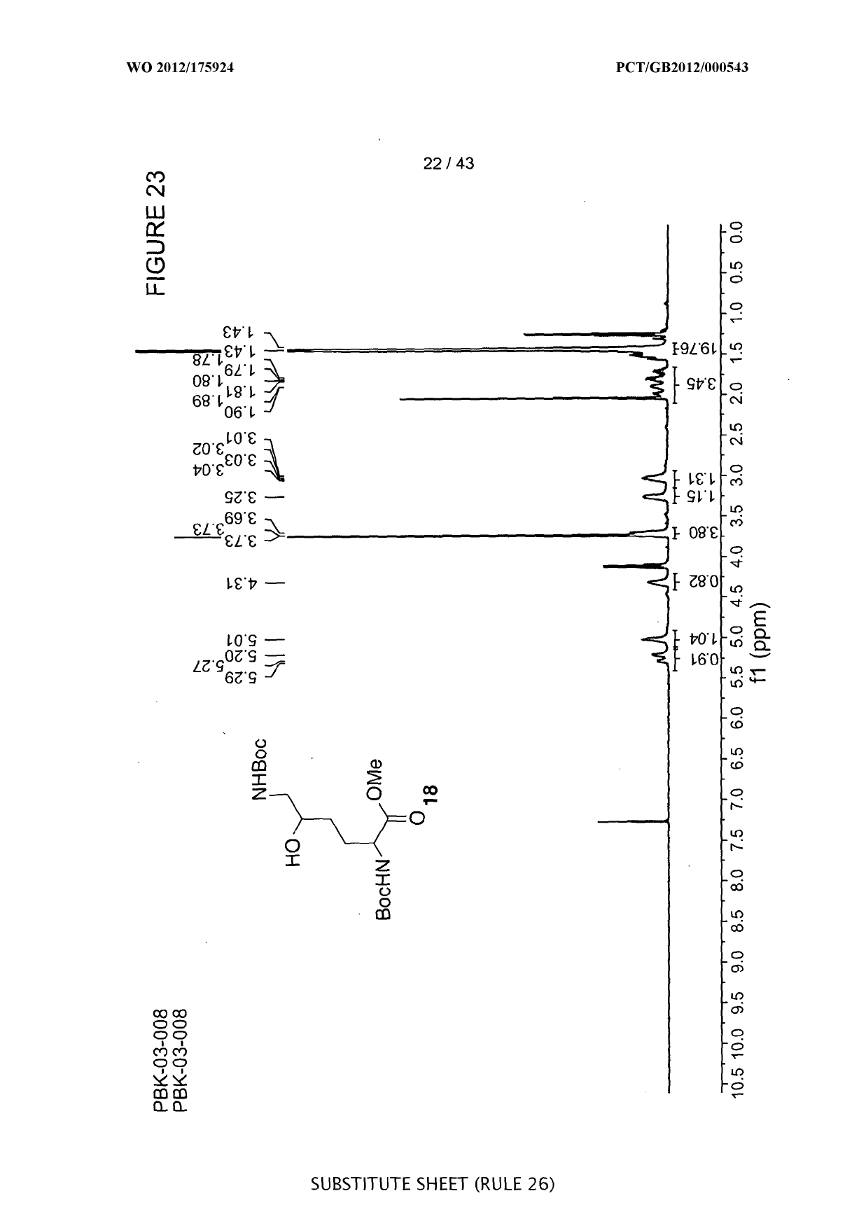

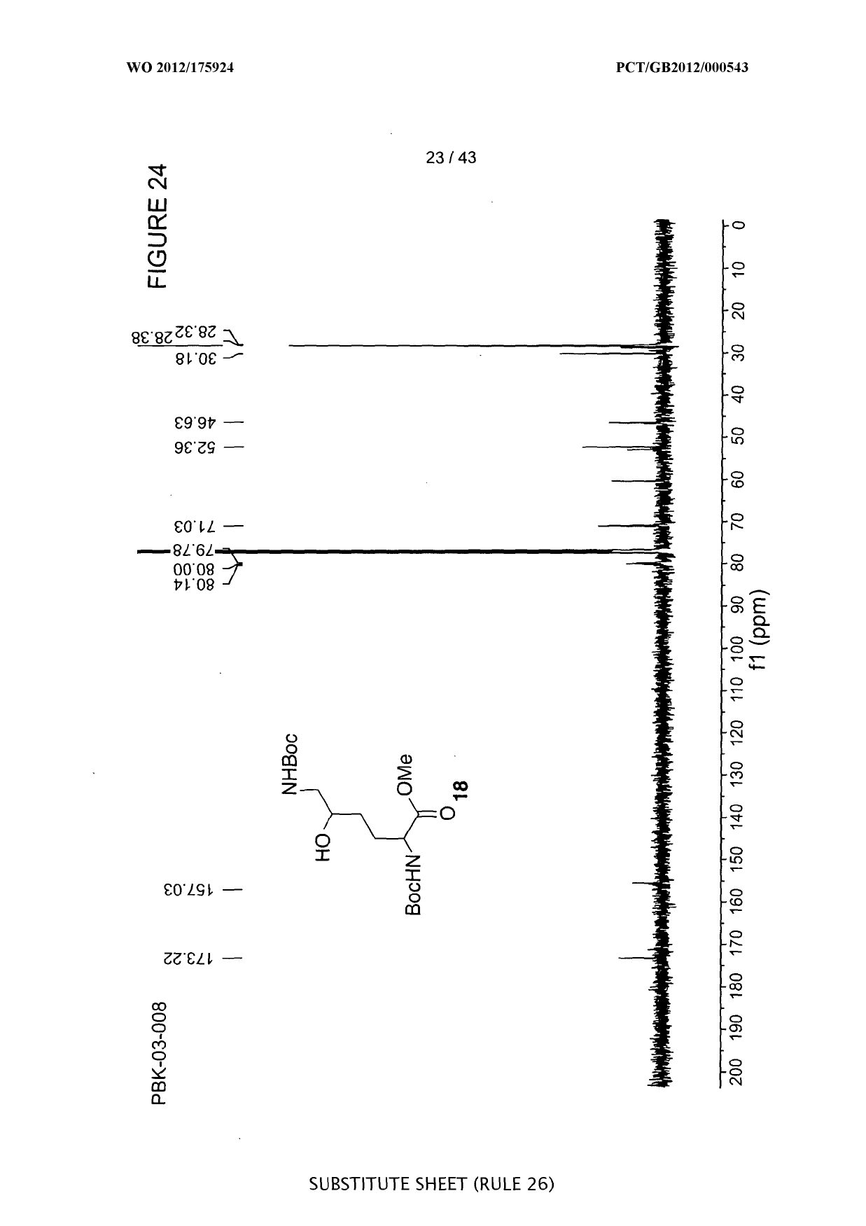

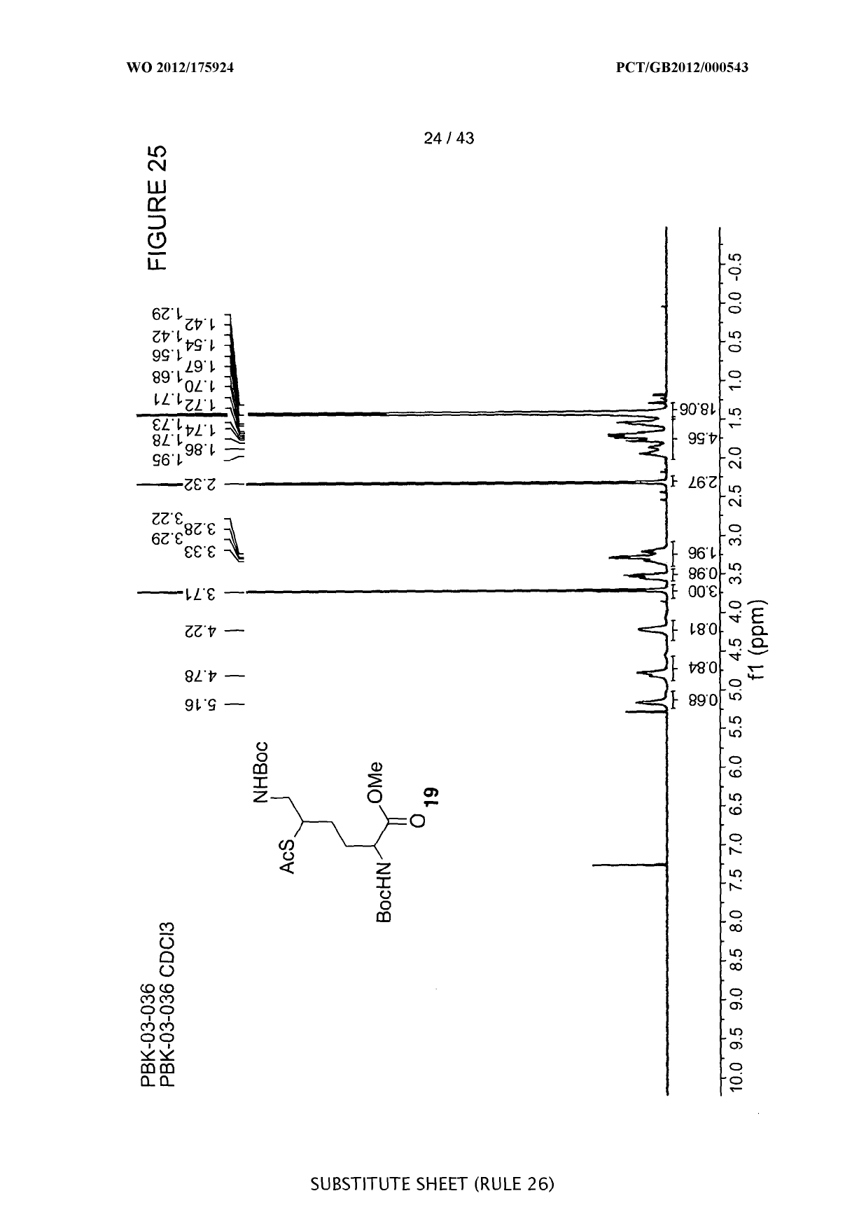

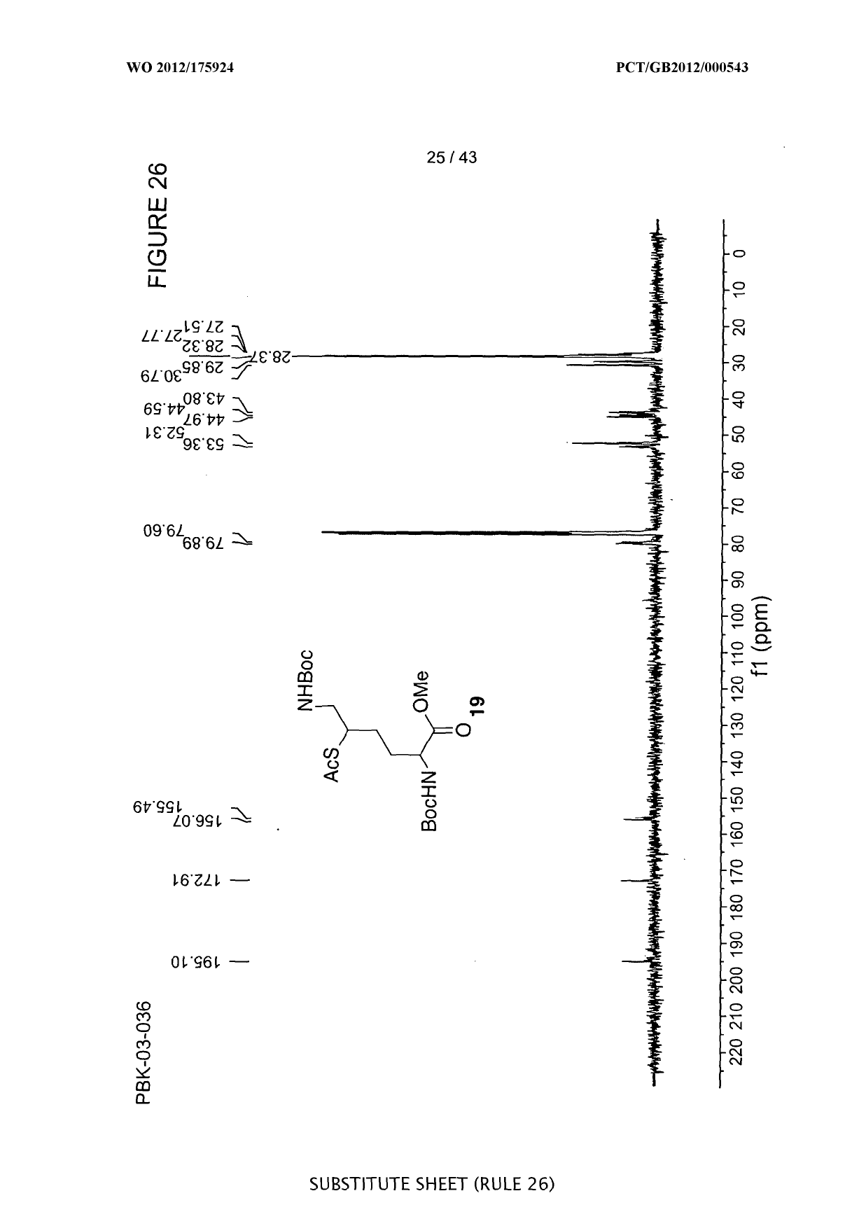

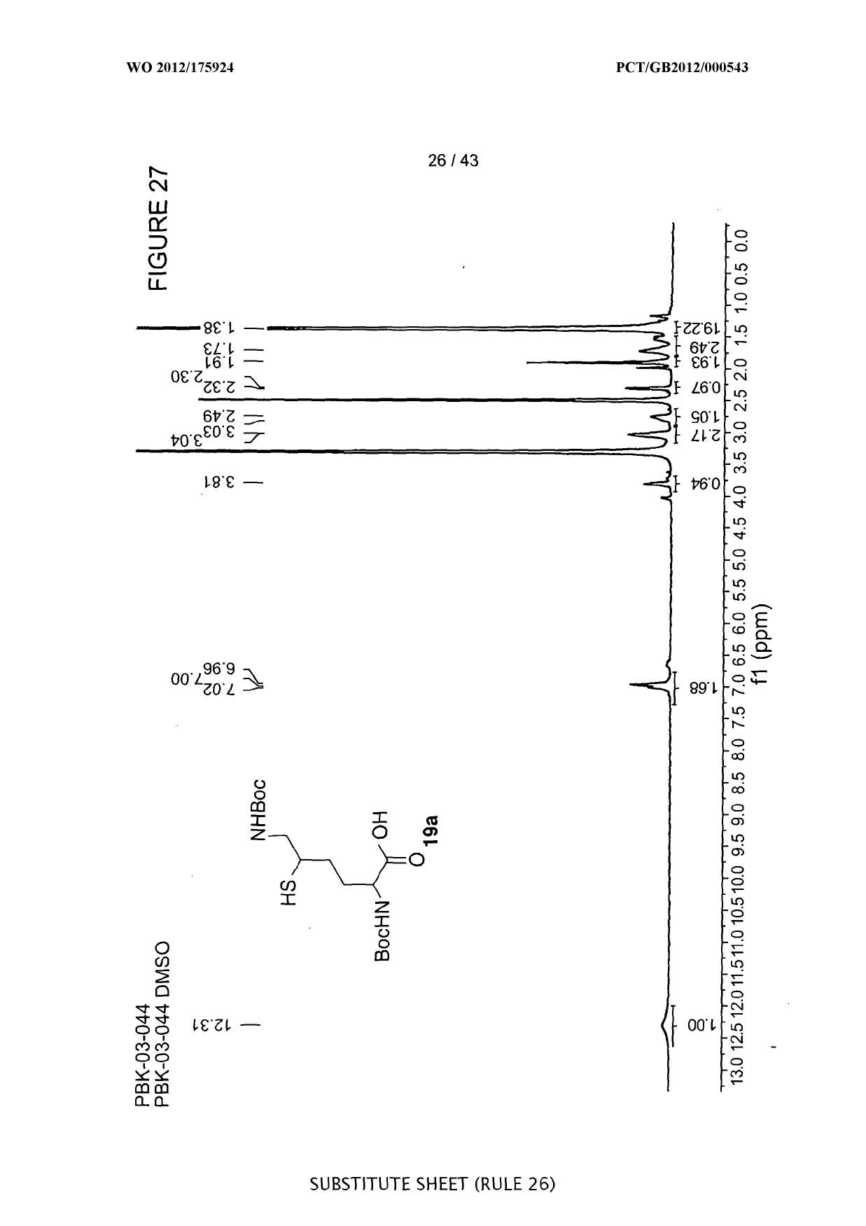

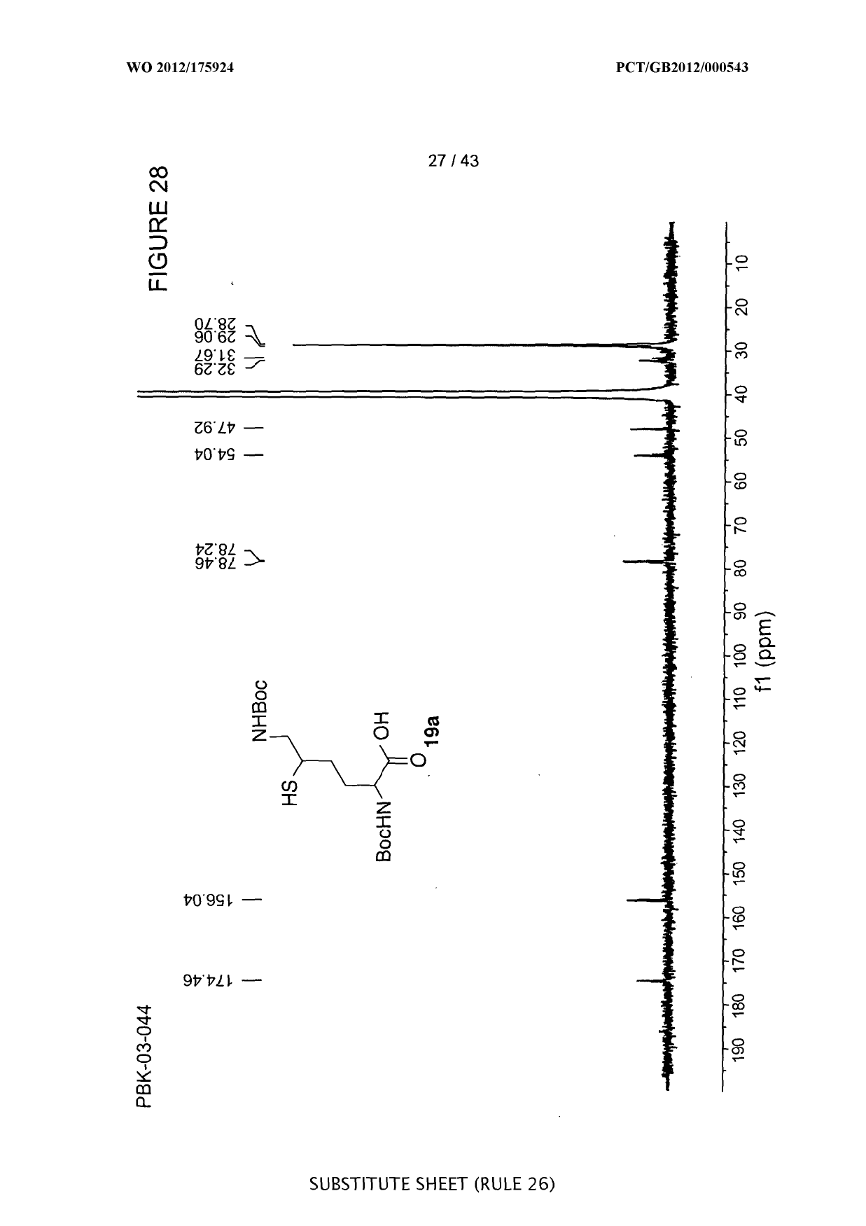

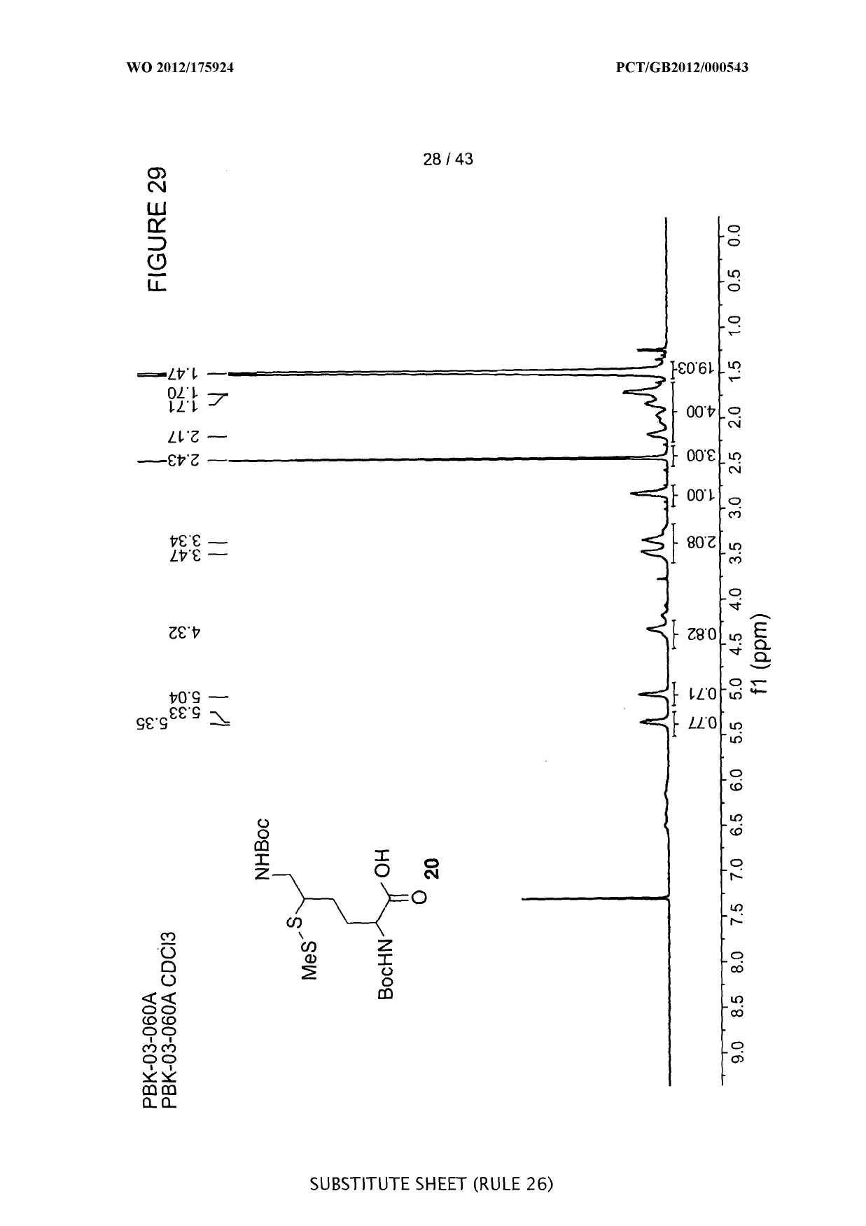

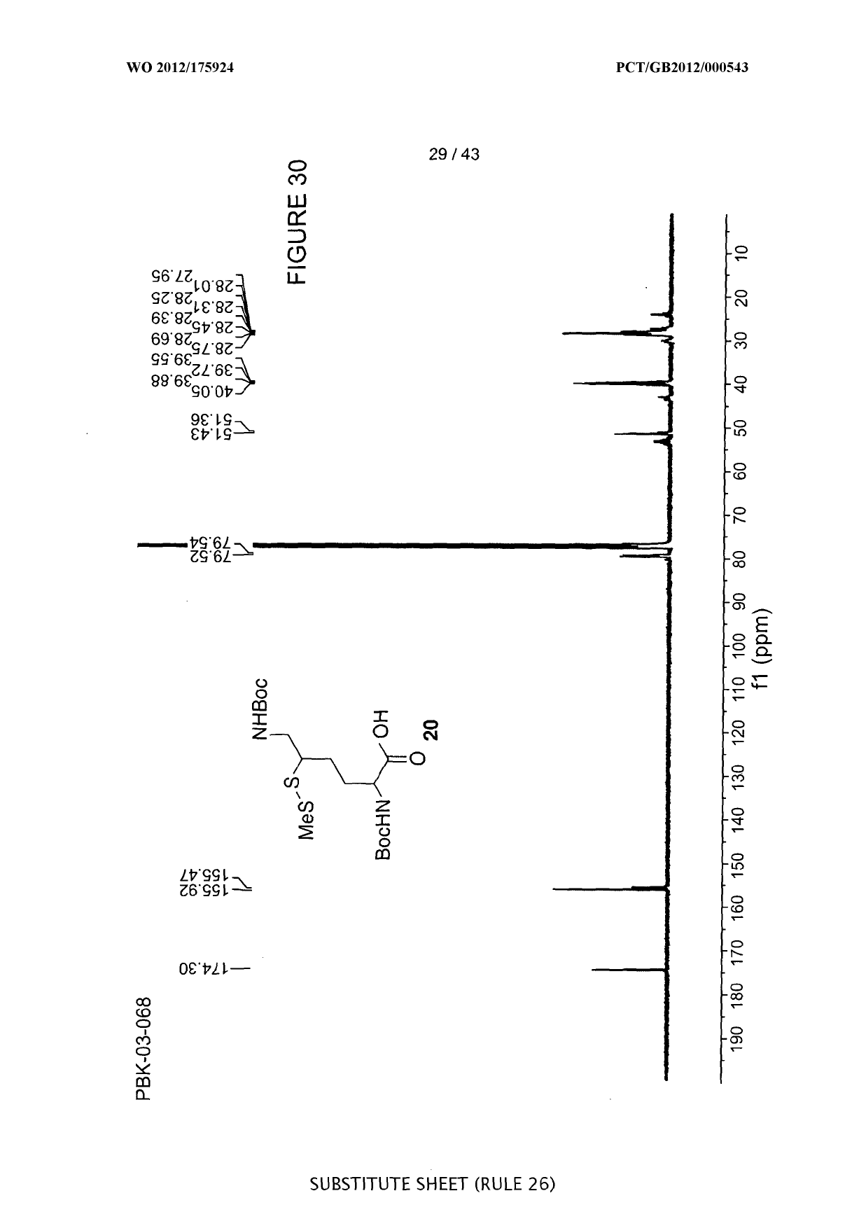

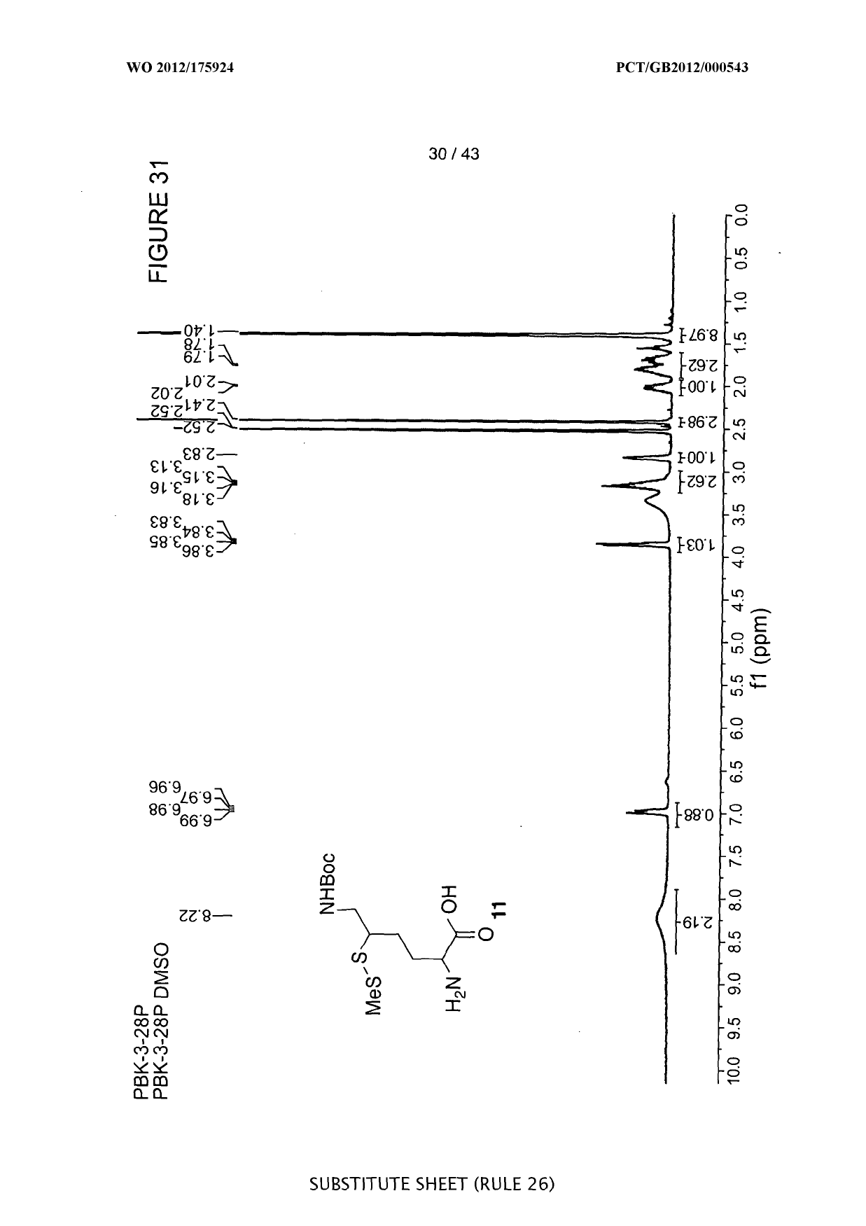

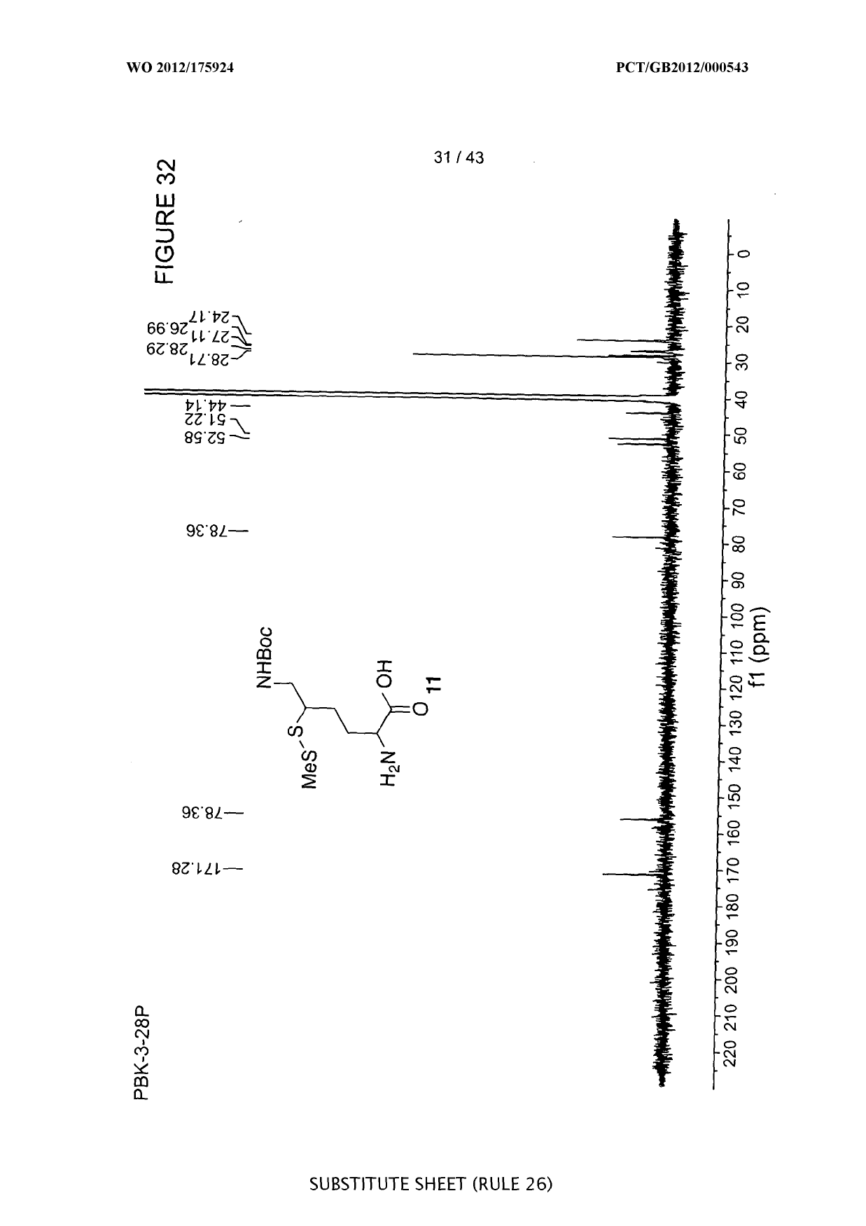

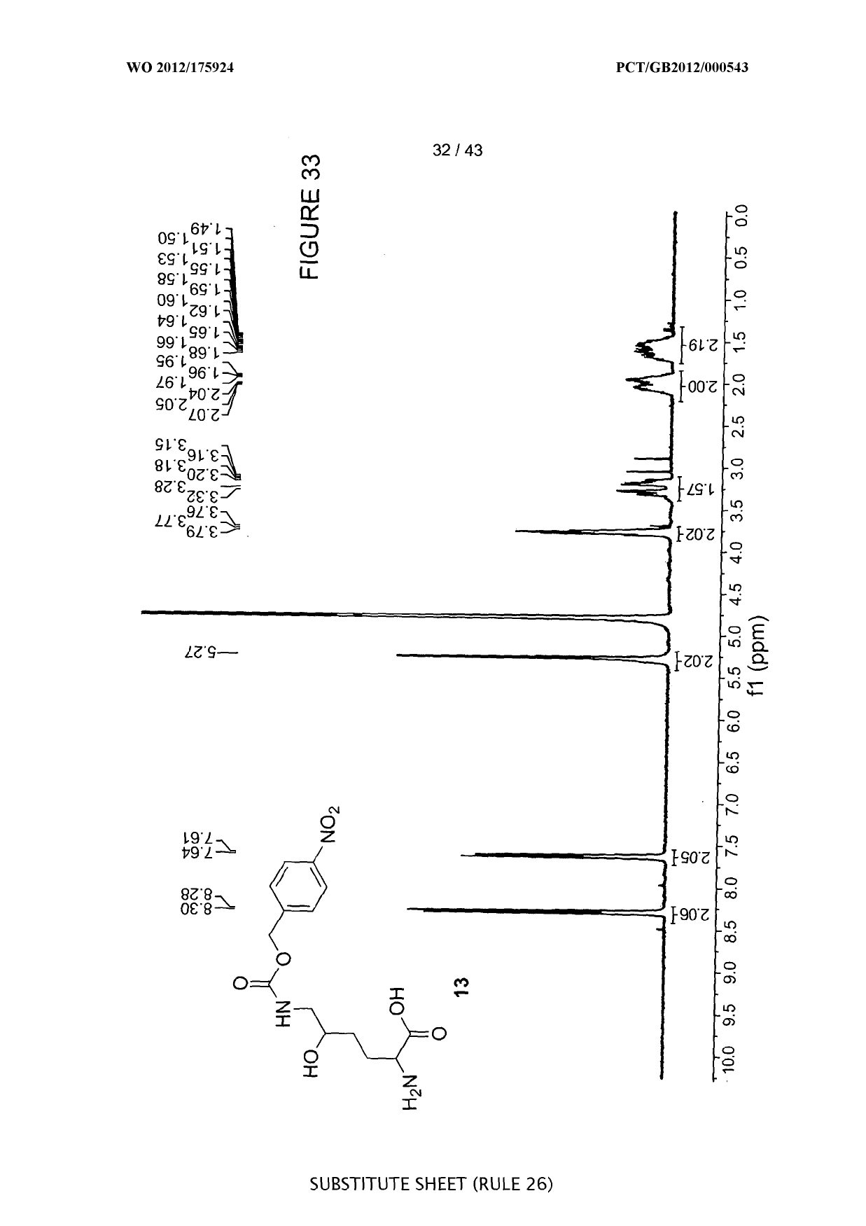

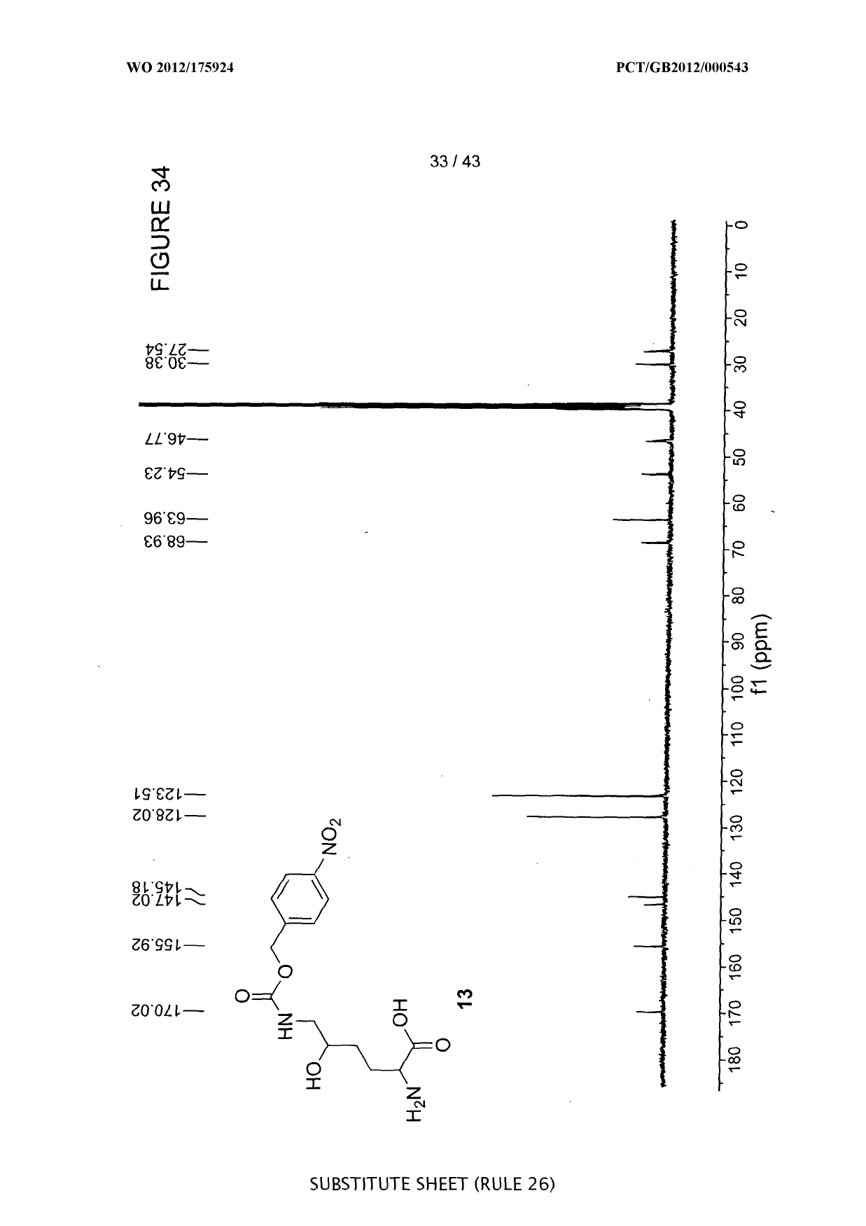

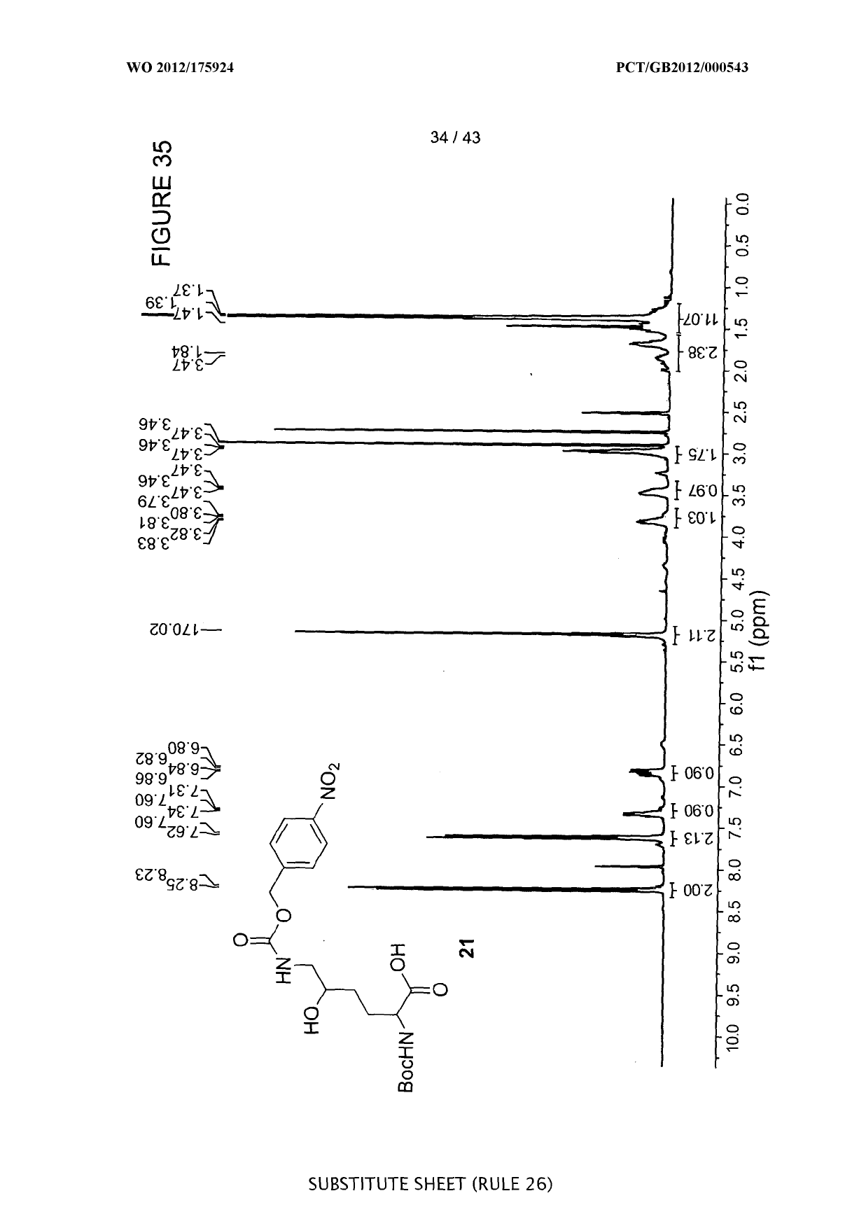

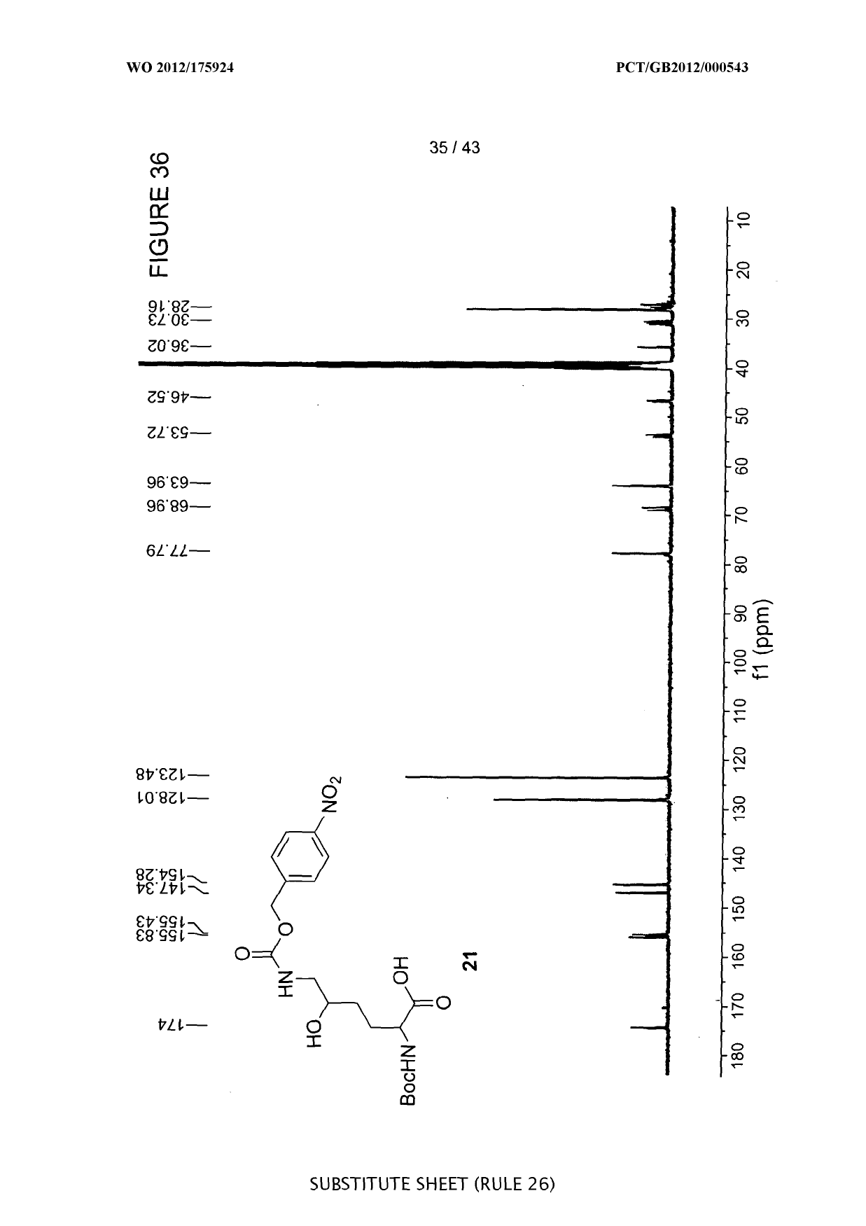

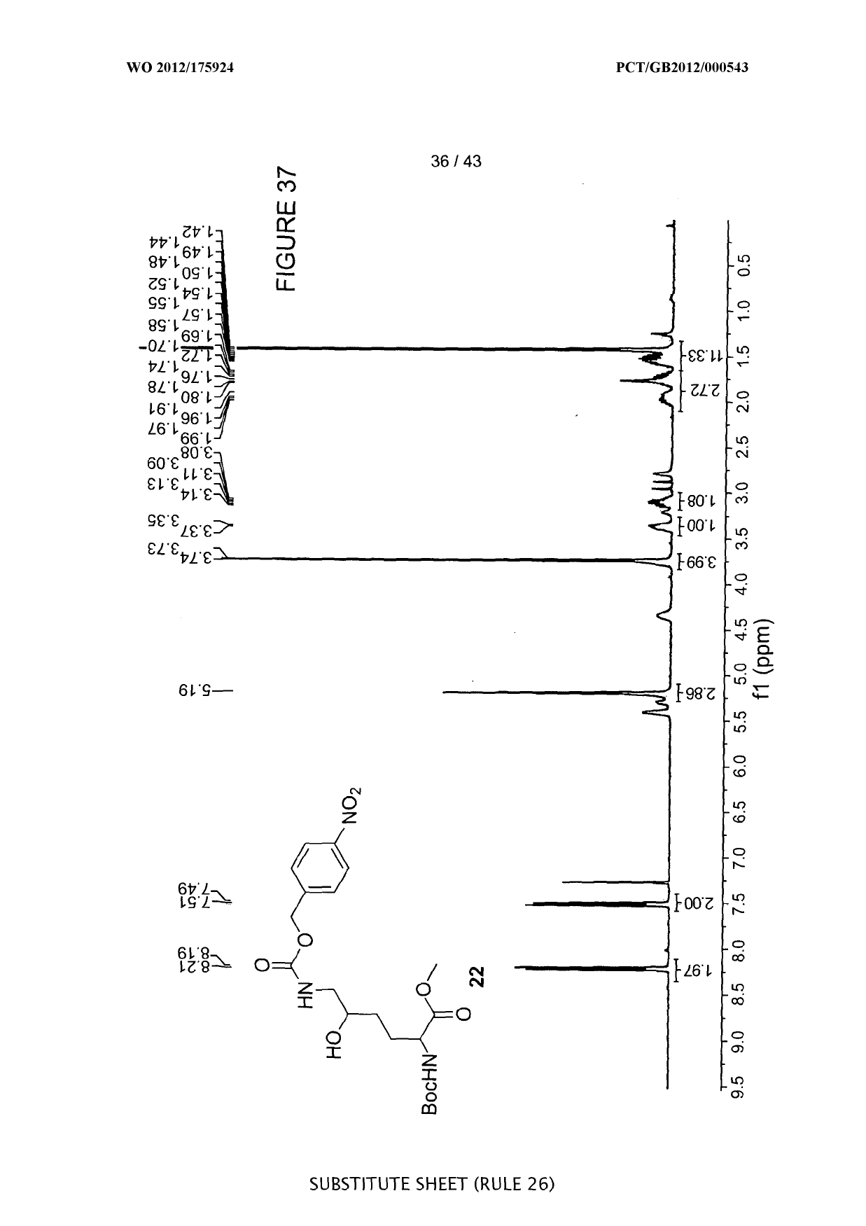

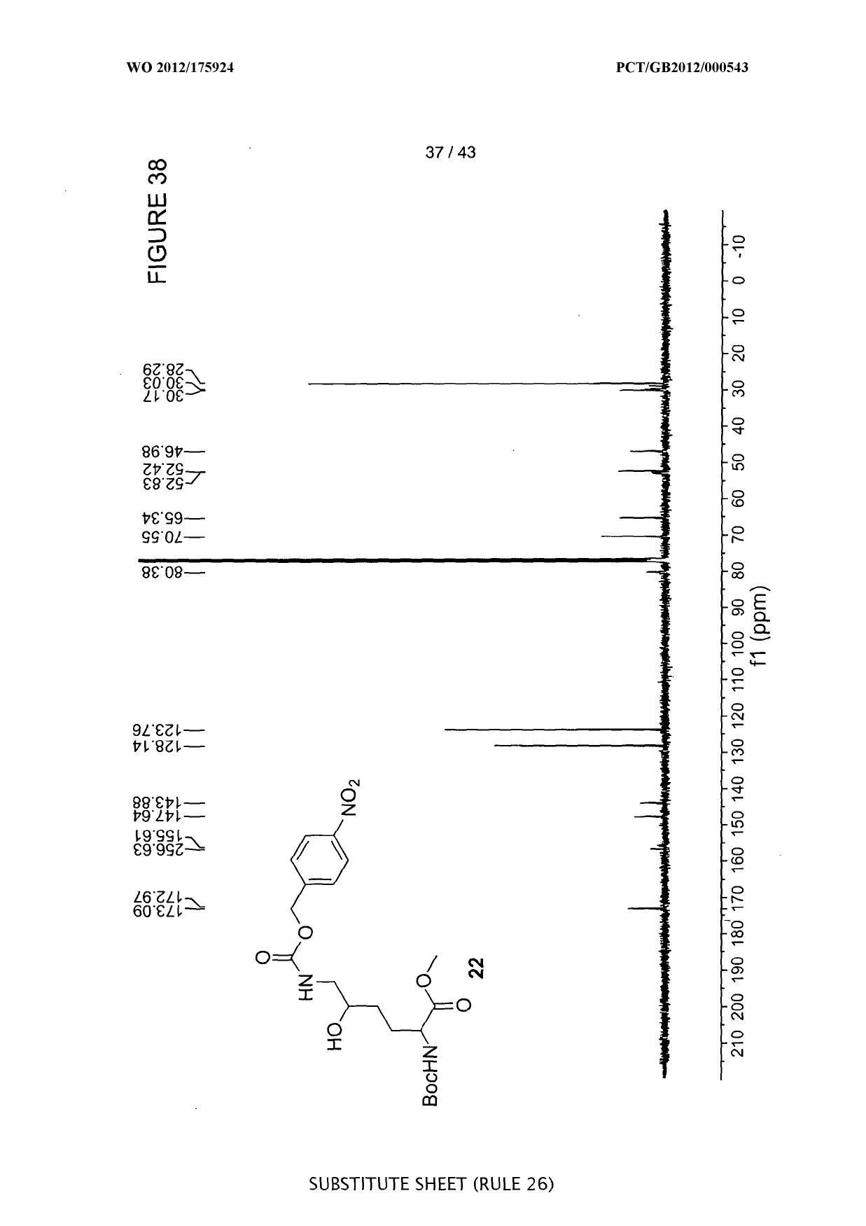

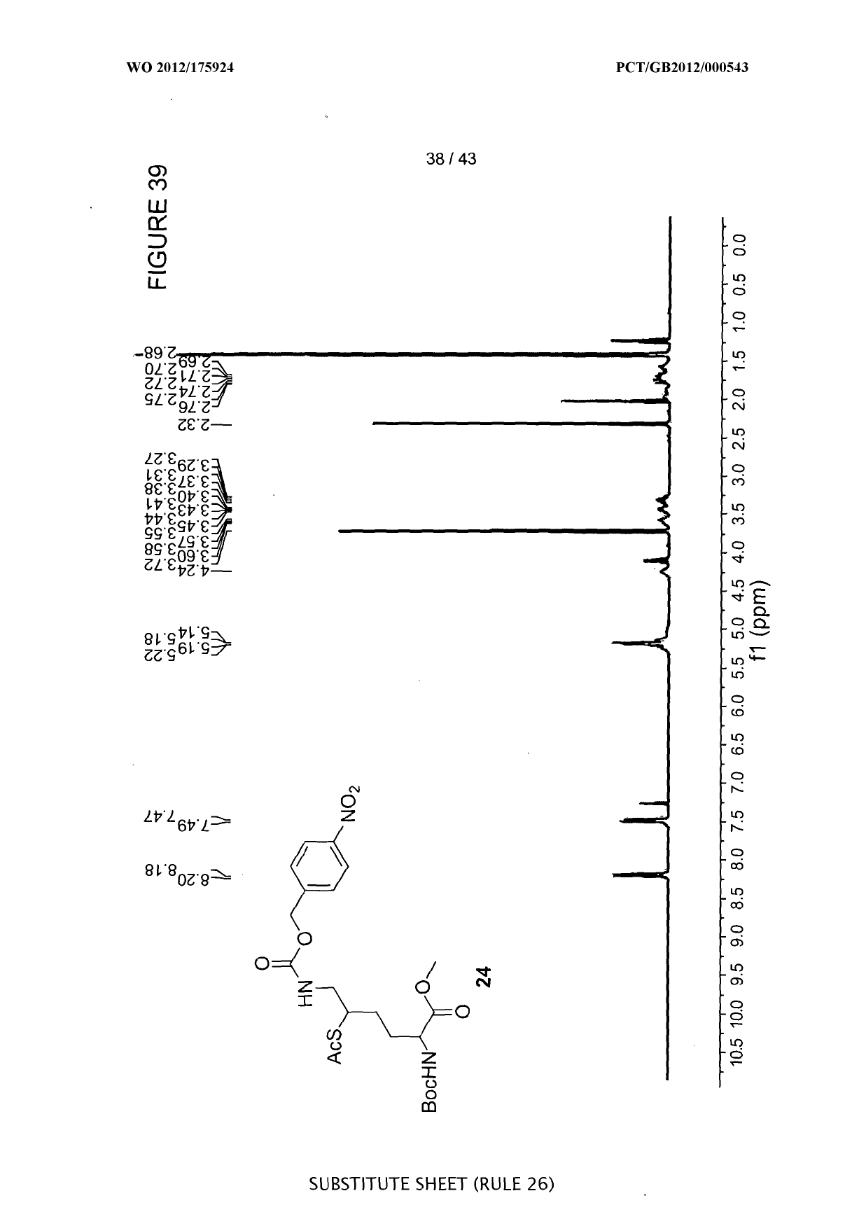

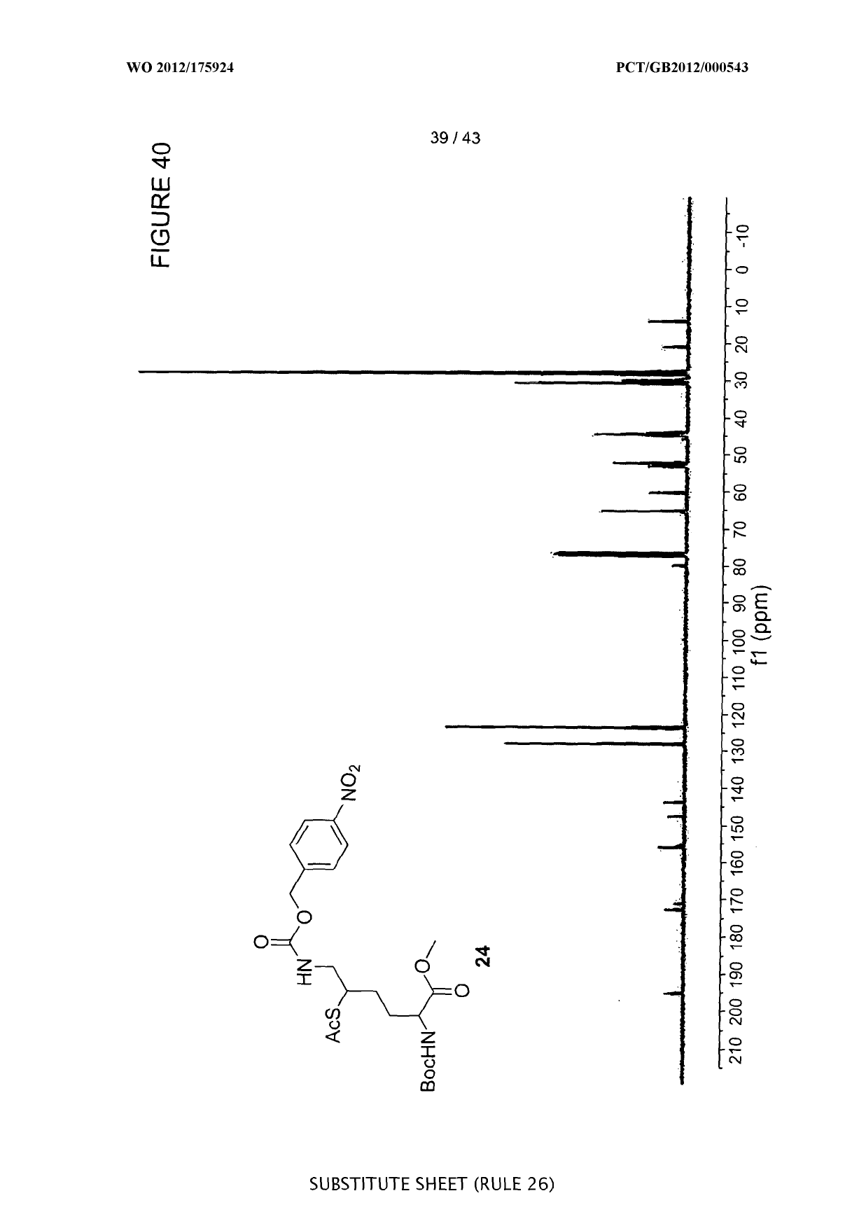

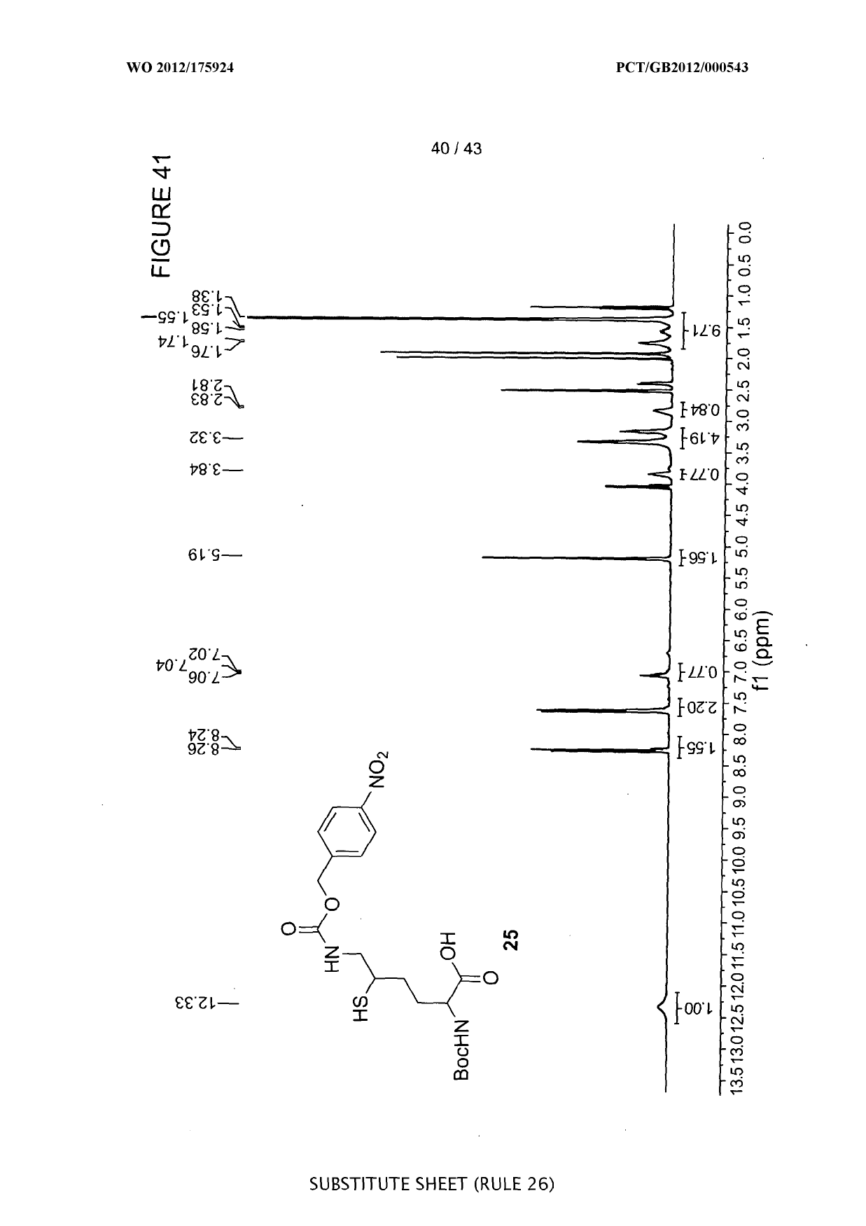

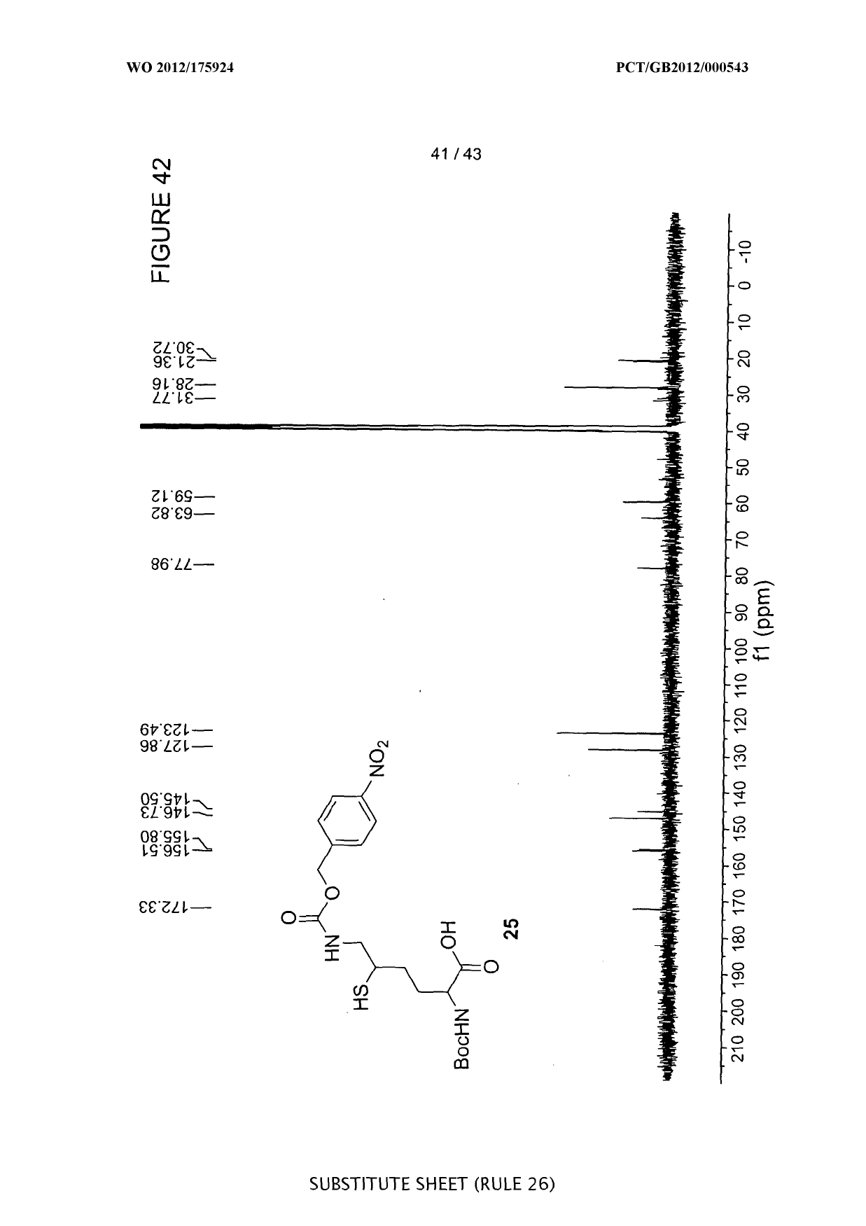

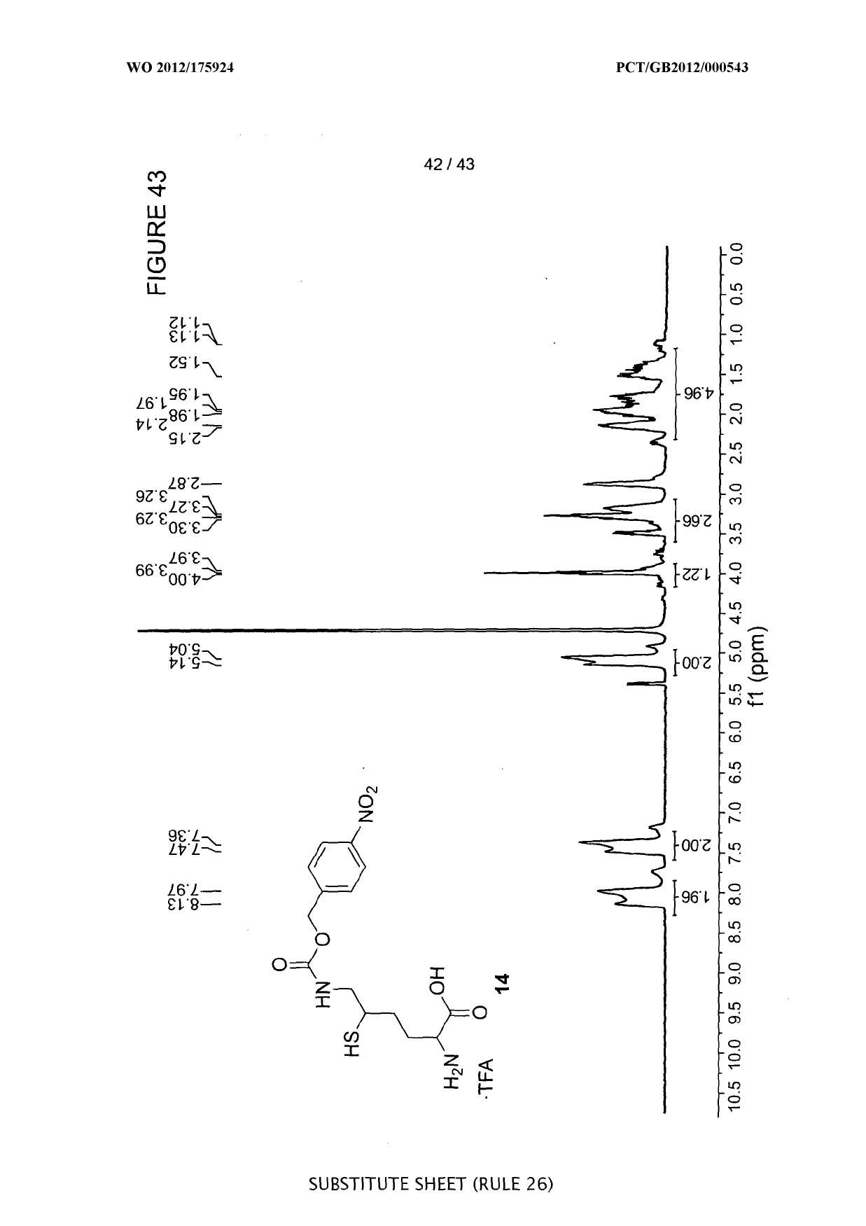

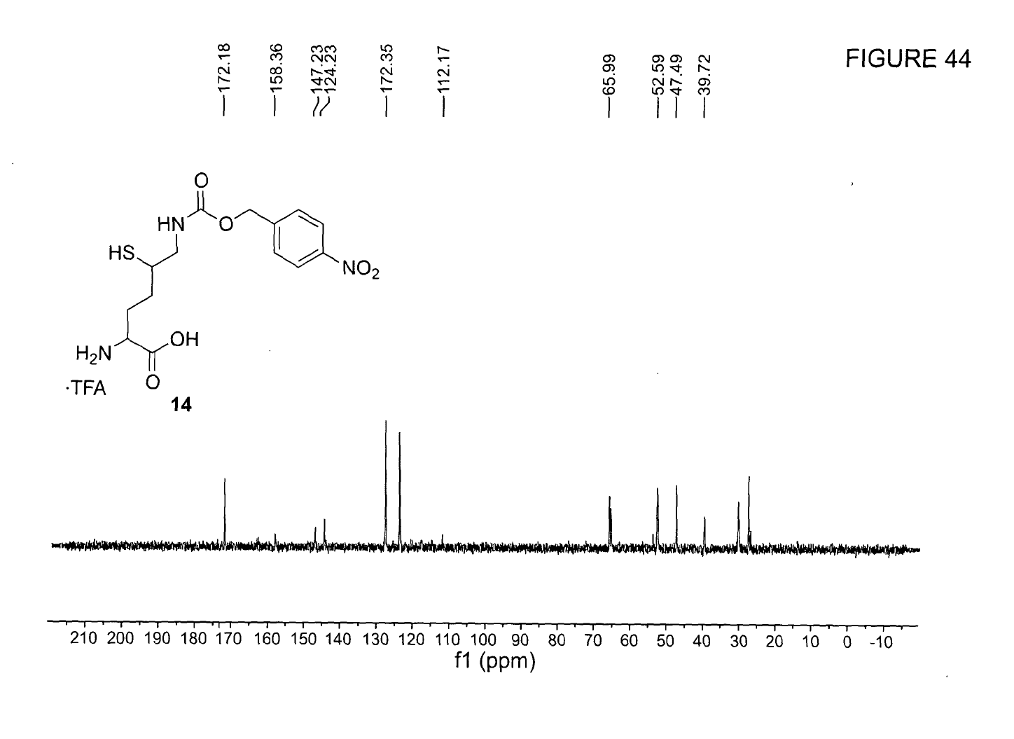

To begin to demonstrate the utility of this system for genetically directing chemoselective protein ubiquitination we synthesized K6-linked diubiquitin—an important ubiquitin linkage that may be involved in DNA repair related signaling processes in mammalian cells 23,24. Ubiquitin bearing 7 at position 6 was dissolved in ligation buffer (200 mM Na2PO4 pH 7.5, 6 M guanidinium chloride (GdmCl), 100 mM mercaptoethonesulfonate (MESNα), 60 mM tris(2-carboxyethyl)phosphine (TCEP). 1.5 equivalents of ubiquitin thioester, prepared by intein fusion thiolysis 10, were added at 25° C. to initiate the reaction. After 48 h SDS-PAGE and LC-MS monitoring (FIG. 3) revealed that approximately 50% of the Ub-His6 containing 7 (Ub6SHK6-His6) had ligated to form the ubiquitin conjugate (DiUbδSHK6-His6). The reaction was diluted to −0.5 mg mL-1 and all ubiquitin species were folded by dialysis against folding buffer (PBS+1 mM DTT). Protein was then buffer exchanged into 50 mM ammonium acetate pH 5. 1 mM 2-mercaptoethanol (BME). The K6-inked diubiqultin conjugate was then purified from residual mono-ubiquitin by ion exchange chromatography (Supplementary FIG. 8) and concentrated to 1 mg mL-1. The purified ubiquitin chain inked via an amide bond between & thiol lysine (7) at position 6 in one ubiquitin and the C-terminus of a second ubiquitin (DiUbδSHK6-His6) was a single band by SDS-PAGE, a single peak by HPLC and had the expected mass confirming the formation of the amide bond (Supplementary FIG. 8). To reveal the entirely native isopeptide bond DiUbδSHK6-His6 was dialyzed into desulfurization buffer (200 mM Na2HPO4, pH 7. 6 M GdmCl, 0.5 mM TCEP). Desulfurization was carried out by the free-radical method 25 upon addition of 250 mM TCEP, 7% 2-dimethly-2-propanethiol and 2.5 mM VA-044, (2,2′-azobis(2-(2-imidazolin-2-yl)propone) dihydrochloride) as radical initiator. Desulfurization was complete after 1 h as determined by LC-MS yielding the first geneticaly directed native isopeptide linkage to ubiquitin (DIUbK6-His6; FIG. 3 & Supplementary FIG. 9). Desutfurizoation reagents were removed and concomitant folding was achieved by dialysis of the reaction mixture against 10 mM Tris, pH 7.6 buffer. To further confirm the biological integrity of the ubiquitin dimers synthesized by our method we demonstrated that they are efficient substrates for members of the ubiquitin specific protease (USP) family of deubiquitinases (USP2 and USP5), that we have previously shown are able to readily hydrolyse K6-linked diubiquitin 10 (Supplementary FIG. 10).

In conclusion we have demonstrated the first route for the efficient site-specific incorporation of δ-thiol lysine (7) and δ-hydroxy lysine (8) into recombinant proteins. We have combined the genetically directed incorporation of 7 with native chemical ligation and desulfurization to yield an entirely native isopeptide bond between substrate proteins and ubiquitin. Moreover, we have discovered that p-nitro carbobenzyloxy group can be used to install lysine and its close analogs into proteins, and this may facilitate the preparation of proteins bearing site specifically isotopically labeled lysines for NMR applications.

We have developed synthetases for 5 new amino acids, including the first for delta substituted lysines. We have also demonstrated that independently selected synthetase mutations that allow the incorporation of epsilon substituted ysine and that allow the incorporation of δ substituted lysines can be combined to incorporate lysine derivatives bearing both δ and ε substituents in a single molecule.

### REFERENCES

(1) Chen, Z. J.; Sun, L. J. Mol. Cel 2009, 33, 275.
(2) Ikeda, F.; Crosetto, N.; Dikic, I. Cel 2010, 143, 677.
(3) Deshaies, R. J.; Joazeiro, C. A. P. Annu Rev Biochem 2009, 78, 399.
(4) Chen, J.; Ai, Y.: Wang, J.; Haracsko, L.: Zhuang, Z. Not Chem Biol 2010, 6, 270.
(5) Chatterjee, C.; McGinty, R. K.; Ferz B.; Muir, T. W. Not Chem Biol 2010, 6, 267.
(6) Shanmugham, A.; Fish. A.; Luna-Vargas, M. P. A.; Faesen, A. C.; El Oualid, F.; Sixma, T. K.; Ovoo, H. J Am Chem Soc 2010, 132, 8834.
(7) Eger, S.; Scheffner, M.; Morx, A.; Rubini, M. J Am Chem Soc 2010, 132, 16337.
(8) Li, X.; Fekner, T.: Ottesen, J. J.; Chan, M. K. Angew Chem Int Ed Engl 2009, 48, 9184.
(9) McGinty, R. K.; Köhn, M.; Chatterjee, C.; Chiang, K. P.; Pratt, M. R.; Muir, T. W. ACS Chem Bio 2009, 4, 958.
(10) Virdee, S.: Ye, Y:. Nguyen, D. P.: Komonder, D.; Chin, J. W. Not Chem Biol 2010, 6, 750.
(11) Chatterjee, C.; McGinty, R. K.; Pelois, J.-P.; Muir, T. W. Angew Chem int Ed Engi 2007, 46, 2814.
(12) McGinty, R.; Kim, J.; Chatterjee, C.; Roeder, R.; Muir, T. Nature 2008, 453, 812.
(13) Yang, R.; Pasunooti, K.; UL, F.; Liu, X.; Uu, C. J Am Chem Soc 2009, 131, 13592.
(14) Yang, R.; Posunooti, K. K.; U, F.; Uu, X.-W.; Liu, C.-F. Chem Commun (Comb) 2010, 46, 199.
(15) Ajish Kumar, K.; Hoj-Yohyo, M.; Oischewski, D.; Loshuel, H.; Bik, A. Angew Chem Int Ed Engi 2009, 48, 8090.
(16) Kumar, K. S. A.; Sposser, L.; Erich, L. A.; Bavikar, S. N.; Brik, A. Angew Chem int Ed Engl 2010, 49, 9126.
(17) El Oualid, F.; Merkx, R.; Ekkebus. R.; Homeed, D. S.; Smit, J. J.; de Jong, A.; Hilkmann, H.; Sixma, T. K.; Ovao, H. Angew Chem Int Ed Engl 2010, 49, 10149.
(18) Yanagisawo. T: Ishii, R.; Fukunogao, R.; Kobayashi, T.; Sakamoto, K.: Yokoyama, S. Chem Biol 2008, 15, 1187.
(19) Nguyen, D.; Garcia Alai, M.; Kapodnis, P.; Neumann, H.; Chin, J. J Am Chem Soc 2009, 131, 14194.
(20) Kavran. J. M.; Gundllapalli. S.; O'Donoghue. P.; Englert, M.; Söll, D.; Steitz. T. A. Proc Natl Acod Sci USA 2007, 104, 11268.
(21) Neumann, H.; Peak-Chew, S.; Chin, J. Nat Chem Biol 2008, 4, 232.
(22) Ottesen, J. J.; Bar-Dagan, M.; Giovani, 8.; Muir, T. W. Biopolymers 2008, 90, 406.
(23) Nishikawa, H.: Ooka, S.: Soto, K.; Arima. K.: Okomoto. J.; Klevit, R.; Fukuda, M.: Ohta, T. J Biol Chem 2004, 279, 3916.
(24) Wu-Baer, F.; Lograzon, K.; Yuan, W.; Boer, R. J Bio Chem 2003, 278, 34743.
(25) Wan, Q.; Danishefsky. S. J. Angew Chem Int Ed Eng 2007, 46, 9248.

All publications mentioned in the above specification are herein incorporated by reference. Various modifications and variations of the described aspects and embodiments of the present invention will be apparent to those skilled in the art without departing from the scope of the present invention. Although the present invention has been described in connection with specific preferred embodiments. It should be understood that the invention as claimed should not be unduly limited to such specific embodiments. Indeed, various modifications of the described modes for carrying out the invention which are apparent to those skilled in the art are intended to be within the scope of the following claims.

### General Methods

Using an Agilent 1200 LC-MS system, ESI-MS was carried out with a 6130 Quadrupole spectrometer. The solvent system consisted of 0.1% formic acid in H₂O as buffer A, and 0.1% formic acid in acetonitrile (MeCN) as buffer B. Protein UV absorbance was monitored at 214 and 280 nm. Protein MS acquisition was carried out in positive ion mode and total protein masses were calculated by deconvolution within the MS Chemstation software (Agilent Technologies). Protein mass spectrometry was additionally carried out with an LCT TOF mass spectrometer (Micromass). Samples were prepared with a C4 Ziptip (Millipore) and infused directly in 50% aqueous acetonitrile containing 1% formic acid. Samples were injected at 20 μL min⁻¹ and calibration was performed in positive ion mode using horse heart myoglobin. 30 scans were averaged and molecular masses were obtained by maximum entropy deconvolution with MassLynx version 4.1 (Micromass).

Small molecule LC-MS was carried out using the Agilent system. Variable wavelengths were used and MS acquisitions were carried out in positive and negative ion modes. Kieselgel 60 F-254 commercial plates were used for analytical TLC, UV light and/or potassium permanganate stain was used to follow the course of the reaction. Flash chromatography (FC) was performed with silica gel grade 9385 pore size 60 Å, 230-400 mesh. The structure of each compound was confirmed by ¹H & ¹³C NMR (500 & 126 MHz, Broker spectrometer). Chemicals shifts (6) are reported in ppm. J values are in hertz, and the splitting patterns are designed as follows: s, singlet; bs, broad singlet; d, doublet; t, triplet; q, quartet; m, multiplet. The mass spectra were obtained on an Agilent 1200 series LC-MS system.

### Library Construction and Selection of Aminoacyl-tRNA Synthetases Specific for Unnatural Amino Acids 9, 10, 12, 13 & 14

Enzymatic inverse PCR was used to generate DNA libraries based on the pBK-PyIRS template¹. The Y349 library was made with the forward 5′-GGAAAGGTCTCGCTGCATGGTGNNKGGCGATACCCTGGATATTATG-3′ primer and the reverse 5′-CAGTAGGTCTCTGCAGCTATCGCCCACAATTTCGAAGTCG-3′ primer. The PCR product was sequentially digested with DpnI and BsaI. Ligating the digested PCR product with T4 DNA ligase generated circularized plasmid DNA. Ligated DNA was ethanol precipitated and used to transform ElectroMAX DH10B electrocompetent cells (Invitrogen) producing 10⁶ transformants. Transformed cells were used to inoculate an LB overnight culture containing kanamycin (50 μg/mL). Cells from the overnight culture (5 mL) were used to miniprep Y349 library DNA. DNA sequencing confirmed randomization of the Y349 codon and sequencing of 10 independent colonies revealed that there was no apparent bias in the library. To select a synthetase that incorporated hydroxyamino acid 9, Y349 library DNA was used to transform eletrocompetent DH10B cells containing the pREP-PyIT plasmid². This plasmid contains a cat gene with an amber codon at a permissive site. Approximately 1000 cells were plated onto an LB agar plate containing tetracycline (12.5 g mL⁻¹), kanamycin (25 μg mL⁻¹), chloramphenicol (50 μg mL⁻¹), and 5 mM 9. 3 colonies were found after overnight growth at 37° C. and these were used to inoculate overnight cultures that were used to miniprep DNA and to inoculate 2 mL LB cultures (1:10) containing tetracycline (12.5 μg mL⁻¹), kanamycin (25 μg mL⁻¹). The cultures were split into 2×1mL volumes and one received 9 (5 mM) and one did not. After growth at 37° C. for 5 h, 1 μL of each culture was spotted onto LB agar plates containing tetracycline (12.5 μg mL⁻¹), kanamycin (25 μg ml⁻¹), with or without 9 (5 mM) and increasing concentrations of chloramphenicol (50-300 μg ml⁻¹). The synthetase plasmids were separated from the reporter plasmid by gel purification. Chemically competent DH10B cells were transformed with the purified plasmid and cells were plated and individual colonies used to inoculate overnight cultures for miniprep and sequencing (GATC Biotech). The best clone (as determined by the phenotyping experiments and small scale protein expressions probed by western blot with anti-His₆ antibody) was named pBK-6SHK.

Selection of aminoacyl-tRNA synthetase (nitroCbzKRS) specific for unnatural amino acid 12 was carried out as previously described³. Amino acid was obtained from Bachem (#E-2960).

Construction of aminoacyl-tRNA synthetase (nitroCbzKRS*) specific for incorporation of amino acids 13 and 14 was achieved by introducing a Y349M mutation into nitroCbzKRS by Quikchange mutagenesis. The resulting synthetase contained the mutations M241, A267, Y27I, L274, C313 and Y349W.

### Synthesis of Amino Acids 9 and 10

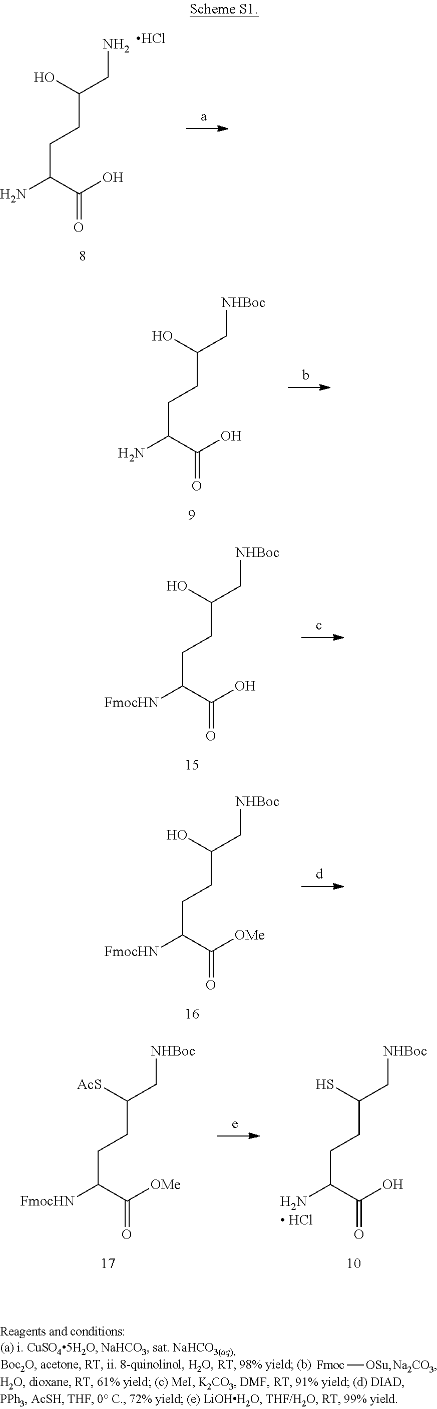

### 2-amino-6-(tert-butoxycarbonylamino)-5-hydroxyhexanol acid (9)

To a stirred solution of 8 (5 g, 25.2 mmol, 1 eq) in a saturated solution of NaHCO, (25 mL) at room temperature was added CuSO₄.5H₂O (3.1 g, 12.5 mmol, 0.5 eq) followed by NaHCO₃ (2.1 g, 25.2 mmol, 1 eq). The mixture was stirred at room temperature for 10 min, after which time a solution of Boc₂O (7.4 g, 32.7 mmol, 1.3 eq) in acetone (30 mL) was added. The reaction was stirred for 18 h, at room temperature, producing a thick blue slurry. Methanol (6 ml) was added to the slurry and mixture stirred for a further 1 h before being filtered. The solid was washed with water and ethyl acetate. The solid was then re-suspended in water (250 ml) and 8-quinolinol (9.5 g, 65 mmol, 2.5 eq) added, and then stirred vigorously for 18 h at room temperature. The resulting green slurry was filtered and the solid washed with water. The filtrate was extracted with ethyl acetate (3×50 ml) and the aqueous phase concentrated to dryness under reduced pressure. This gave 9 as a colourless solid (6.5 g, 98% yield).

RP-HPLC rt-2.71 min; δ_{H} (500 MHz, D₄-acetic acid) 4.20-4.06 (1H, m, α-H), 3.87-3.48 (1H, m, δ-H), 3.37-3.23 (1H, m, ε-CHₐH_{b}), 3.21-3.11 (1H, m, ε-CHₐH_{b}), 2.26-2.02 (2H, m, γ-CHₐH_{b}), 1.85-1.58 (2H, m, β-CHₐH_{b}), 1.57-1.37 (10H, m, boc-H); LRMS m/z (ES⁺) 263[M+H]⁺; m/z (ES⁻) 261 [M−H]⁻.

### 1-(9H-fluoren-9-yl)-8-hydroxy-13,13-dimethyl-3,11-dioxo-2,12-dioxa-4,10-diazatetradecane-5-carboxylic acid (15)

9 (6.5 g, 25.0 mmol, 1 eq) was dissolved in water (60 mL), to this solution was added Na₂CO₃ (2.4 g, 22.5 mmol, 0.9 eq). The mixture was stirred at room temperature for 5 min, before the addition of a solution of Fmoc-OSu (7.6 g, 22.5 mmol, 0.9 eq) in dioxane (100 mL). The reaction was stirred at room temperature for 18 h before being concentrated to dryness under reduced pressure. The crude material was partitioned between CH₂Cl₂ (250 mL) and water (250 mL) resulting in a considerable amount of product precipitation. The solid product was filtered and no further purification was required on this material. The aqueous layer of the clarified filtrate was adjusted to pH 2 using 1M HCl. The layers were separated and the aqueous layer extracted with CH₂Cl₂ (3×100 mL). The combined organic fractions were dried (Na₂SO₄), filtered, and concentrated to dryness. The remaining product was recrystallised from CH₂Cl₂ and combined with the first precipitated material, giving 15 (7.5 g, 61% yield) as a colourless solid.

RP-HPLC rt-14.39 min; δ_{H} (500 MHz, d6-DMSO) 7.90 (21, d, J 7.4, fmoc-ArH), 7.74 (2H, d, J 7.3, fmoc-ArH), 7.43 (21H, app. t*, J 7.3, fmoc-ArH), 7.35 (2H, app. t*, J 7.2, fmoc-ArH), 6.74-6.60 (1H, m, fmoc-H), 4.34-4.16 (3H, m, α-H+ε-CHₐH_{b}), 3.99-3.86 (1H, m, δ-H), 3.00-2.81 (2H, m, γ-CHₐH_{b}), 1.97-1.52 (2H, m, β-CHₐH_{b}), 1.52-1.28 (10H, m, boc-H); LRMS m/z (ES⁺) 385 [M-Boc]⁺, 507[M+Na]⁺; m/z (ES⁻) 483 [M−H]⁻.

### methyl 1-(9H-fluoren-9-yl)-8-hydroxy-13,13-dimethyl-3,11-dloxo-2,12-dioxa-4,10-dlazatetradecane-5-carboxylate (16)

15 (7.5 g, 15.4 mmol, 1 eq) was dissolved in dry DMF (130 mL), K₂CO (4.3 g, 31.0 mmol, 2 eq) was added to the solution followed by MeI (3.3 g, 1.3 mL, 23 mmol, 1.5 eq). The reaction was stirred at room temperature for 3 h. The reaction mixture was diluted with ethyl acetate (150 mL) and washed with 1M HCl (2×75 mL) followed by sat. NaHCO₃ (2×75 mL). The organic component was dried (Na₂SO₄), filtered and concentrated under reduced pressure. The crude material was purified by flash chromatography (SiO₂) eluting with ethyl acetate and hexane (60:40). This gave 16 (7.0 g, 91% yield) as a colourless solid.

RP-HPLC rt-15.14 min; δ_{H} (500 MHz, CDCl₃) 7.67 (2H, d, J 7.6, fmoc-ArH), 7.57-7.46 (2H, m, finoc-ArH), 7.32 (2H, app. t*, J 7.3, fmoc-ArH), 7.24 (2H, app. t, J 7.1, fmoc-ArH), 5.61-5.42 (1H, m, fmoc-H), 4.89 (1H, br s, 6-H), 4.39-4.27 (2H, m, s-CHₐH_{b}), 4.14 (1H, t, J 6.6, α-H), 3.66 (3H, s, CH), 3.25-3.10 (1H, m, γ-CHₐH_{b}), 3.03-2.89 (1H, m, γ-CHₐH_{b}), 2.01-1.83 (1H, m, β-CHₐH_{b}), 1.83-1.64 (1H, m, β-CHₐH_{b}), 1.50-1.27 (10H, m, boc-H); δ_{C} (125 MHz, CDC₃) 172.9, 157.2, 156.2, 143.9, 143.8, 141.3, 127.8, 127.1, 125.2, 125.1, 120.1, 119.9, 79.9, 71.6, 71.1, 70.6, 67.1, 54.7-51.7 (m, diastereomers), 47.9-46.2 (m, diastereomers), 31.1-27.4 (m, diastereomers); LRMS m/z (ES⁺) 399 [M-Boc]⁺, 521[M+Na]⁺; m/z (ES⁻) 543 [M+HCO₂⁻]⁻.

### methyl 8-(acetylthio)-1-(9H-fluoren-9-yl)-13,13-dimethyl-3,11-dioxo-2,12-dioxa-4,10-diazatetradecane-5-carboxylate (17)

To a solution of triphenylphosphine (7.3 g, 28.0 mmol, 2 eq) in dry THF (70 mL) at 0° C., was added DIAD (5.7 g, 5.5 mL, 28.0 mmol, 2 eq). This mixture was stirred for 30 min at 0° C. before a solution of 16 (7.0 g, 14.0 mmol, 1 eq) and thioacetic acid (2.1 g, 2.0 mL, 28 mmol, 2 eq) in dry THF (35 mL) was added via cannular. The reaction was stirred at 0° C. for 1 h, then diltuted with ethyl acetate (100 mL) and washed with sat. NaHCO₃ (2×75 mL). The organic component was dried (Na₂SO₄), filtered and concentrated under reduced pressure. The crude material was purified by flash chromatography (SiO₂) eluting with ethyl acetate in CH₂Cl₂ (0-15%). This gave 17 as a colourless gum (5.6 g, 72% yield).

R_{f} 0.25 (hexane/ethyl acetate 70:30); rp-HPLC rt 9.74 min; δ_{H} (500 MHz, CDCl₃) 7.69 (2H, d, J 7.7, fmoc-ArH), 7.54 (2H, d, J 5.7, fmoc-ArH), 7.33 (2H, app. t*, J 7.5, fmoc-ArH), 7.25 (2H, app. t, J 7.3, fmoc-ArH), 4.77-4.64 (1H, m, fmoc-H), 4.34-4.22 (2H, m, ε-CHₐH_{b}), 4.15 (1H, t, J 6.7, α-H), 3.68 (3H, s, OCH₃), 3.55-3.46 (1H, m, δ-H), 3.35-3.09 (2H, m, β-CHₐH_{b}), 2.01-1.83 (1H, m, β-CHₐH_{b}), 2.27 (3H, s, COCH₃) 1.59-1.45 (3H, m, γ-CHₐH_{b}+boc-H), 1.43-1.30 (9H, m, boc-H); LRMS m/z (ES⁺) 457 [M-Boc)]; m/z (ES) 601 [M+HCO₂⁻]⁻, 591.5[M+Cl⁻]⁻.

### 2-amino-6-(ter-butoxycarbonylamino)-5-mercaptohexanole acid hydrochloride (10)

17 (1.38 g, 2.5 mmol, 1 eq) was dissolved in degassed THF:H₂O (3:1, 25 mL), to this solution was added lithium hydroxide monohydate (312 mg, 7.5 mmol, 3 eq) at room temperature under an atmosphere of argon gas. The reaction was stirred at room temperature for 3 h, after which time complete consumption of the starting material was observed by HPLC/MS analysis. The reaction mixture was diluted with water (25 mL) and neutralised by the addition of 1 M HCl (8 mL). The mixture was further acidified to pH 3-4 by the cautious addition of 1M HCl (approx. 2 mL) and washed with ethyl acetate (3×25 mL). The aqueous layer was concentrated under reduced pressure, to give the HCL salt of 10 (776 mg, 99% yield).

δ_{H} (500 MHz, D₂O) 3.78 (1H, t, J 5.5, α-H), 3.27 (1H, dd, J 13.9, 5.2, ε-CHₐH_{b}), 3.15 (1H, dd, J 13.9, 7.1, ε-CHₐH_{b}), 2.96-2.85 (1H, m, δ-H), 2.21-1.87 (2H, m, γ-CHₐH_{b}), 1.86-1.70 (1H, m, βCHₐH_{b}), 1.59-1.33 (11H, m, β-CHₐH_{b}+boc-H); δ_{C} (125 MHz, D₂O) 174.21 (C═O), 158.32 (C═O), 81.31, 54.90, 54.5-54.2 (m, diastereomers), 47.15, 40.1, 31.1-30.1 (m, diastereomers), 28.5-26.9 (m, diastereomers); LRMS m/z (ES) 279 [M+H]⁺; m/z (ES⁻) 277 [M−H]⁻, 555 [2M−H]⁻.

### Synthesis of Amino Acid 11

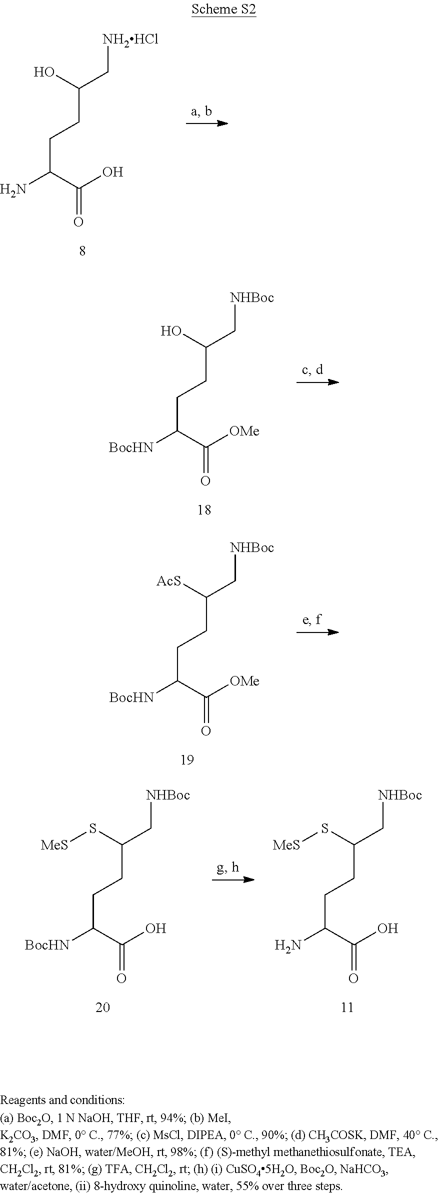

### Methyl 2,6-bis((tert-batoxycarbonyl)amino)-5-hydroxyhexanoate (18)

8 (20 g, 101.0 mmol) was dissolved in 1 N NaOH (200 mL) and Di-tert-butyl dicaibonate (48.3 g, 221.0 mmol) in THF (200 mL) was added drop-wise. The reaction mixture was stirred at rt for 7 h. The complete consumption of the starting material was observed by LC-MS. The mixture was concentrated to 200-250 mL and extracted with ethyl acetate (2×100 mL). The aqueous solution was cooled and acidified to pH 4 with 1 N HCl solution and extracted with ethyl acetate (3×150 mL). The organic layer was dried over anhydrous Na₂SO₄, filtered, concentrated in vacuo and purified by column chromatography (5-15% MeOH in CH₂Cl₂) to yield 2,6-bis((tert-butoxycarbonyl)amino)-5-hydroxyhexanoic acid as a white solid (29.5 g, 81%)

¹H NMR (500 MHz, DMSO) δ 6.59 (bs, 1H), 6.27 (m, 1H), 3.71 (s, 1H), 3.39 (s, 1H), 3.02-2.67 (m, 2H), 1.65 (m, J=76.7 Hz, 3H), 1.30 (bs, 19H). ¹³C NMR (126 MHz, DMSO) δ 176.30, 156.17, 155.53, 77.96, 69.91, 55.42, 47.01, 31.37, 30.99, 28.73. LC-MS: m/z 385.3[M+Na]⁺, 361.3 [M−H]⁻, rt 9.2 min

To a solution of 2,6-bis((tert-butoxycarbonyl)amino)-5-hydroxyhexanoic acid 8a (3.4 g, 9.38 mmol) in DMF (42 mL), solid K₂CO₃ (1.95 g, 14.07 mmol) was added at 0° C. The resulting white suspension was stirred for 5 min before addition of methyl iodide (1.76 mL, 28.1 mmol). The reaction mixture was stirred for 20 h and the progress of the reaction was monitored by LC-MS. The reaction mixture was filtered through celite and the filtrate was partitioned between ethyl acetate (100 mL) and distilled water (100 mL). The ethyl acetate layer was further extracted with water (2×100 mL), dried over Na₂SO₄, filtered and concentrated in vacuo to give a pale yellow viscous oil, which was purified by flash column chromatography using ethyl acetate:hexane (1:1) to yield 18 (2.73 g, 77%)

¹H NMR (500 MHz, CDCl₃) δ 5.41-5.10 (m, 1H), 5.01 (bs, 1H), 4.31 (bs, 1H), 3.72 (s, 3H), 3.69 (bs, 1H), 3.25 (d, J=7.7 Hz, 1H), 3.02 (dd, J=12.7, 6.5 Hz, 1H), 2.10-1.65 (m, 3H), 1.43 (bs, 19H). ¹³C NMR (126 MHz, CDCl₃) δ 173.22, 157.03, 80.00, 79.78, 71.03, 60.42, 52.36, 46.63, 30.18, 28.38, 28.32. LC-MS: m/z 377.3 [M+H]*, rt 9.7 min

### Methyl 5-(acetylthio)-2,6-bis((tert-butoxycarbonyl)amino)hexanoate (19)

To a solution of 18 (2.6 g, 6.91 mmol) in dichloromethane (30 mL), N,N-diisopropylethylamine (DIPEA) (2.41 mL, 13.81 mmol) was added. The solution was cooled to 0° C. and methanesulfonyl chloride (0.657 mL, 8.43 mmol) was added drop-wise. The reaction mixture was stirred at 0° C. for 1 h, allowed to reach room temperature and then stirred for a further 1 h. The reaction mixture was diluted with dichloromethane (100 mL), washed with saturated ammonium chloride solution and then with brine. The organic layer was dried over NazSO₄, filtered and concentrated in vacuo to give a light brown foam, methyl 2,6-bis((tert-butoxycarbonyl)amino)-5-((methylsulfonyl)oxy)hexanoate (3.10 g, 99%), which was directly used for the next step. LC-MS: m/z 477.2 [M+Na]⁺, 499.2[M+HCO₂]⁻, rt 10.4 min

Methyl 2,6-bis((tert-butoxycarbonyl)amino)-5-((methylsulfonyl)oxy)hexanoate (3.10 g, 6.82 mmol) was dissolved in DMF (120 mL) and potassium thioacetate (2.37 g, 20.86 mmol) was added. The reaction mixture was stirred at 40° C. for 16 h and allowed to reach room temperature. Water (150 mL) was added to the reaction mixture and extracted with ethyl acetate (3×150 mL). The organic layer was washed with water and brine, dried over Na₂SO₄, filtered and concentrated in vacuo to give a light brown oil. The crude compound was purified by flash column chromatography using ethyl acetate:hexane (1:2) to yield 19 (2.30 g, 78%).

¹H NMR (500 MHz, CDCl₃) δ 5.16 (s, 1H), 4.78 (s, 1H), 4.22 (s, 1H), 3.71 (s, 3H), 3.53 (dt, J=12.8, 7.1 Hz, 1H), 3.43-3.09 (m, 2H), 2.32 (s, 3H), 2.04-1.49 (m, 4H), 1.42 (d, J=1.1 Hz, 18H). ¹³C NMR (126 MHz, CDCl₃) δ 195.10, 172.91, 156.07, 155.49, 79.89, 79.60, 53.36, 52.31, 44.97, 43.80, 30.79, 29.85, 28.37. LC-MS: m/z 458.3[M+Na]⁺, 479.2 [M+HCO₂]⁻, rt 10.8 min

### 2,6-bis((tert-butoxycarbonyl)amino)-5-(methyldisulfanyl)hexanoic acid (20)

To a solution of 19 (2.1 g, 4.83 mmol) in methanol (25 mL), a solution of NaOH (0.58 g, 14.50 mmol) in water (25 mL) was added at rt and the reaction mixture was stirred for 1 h. The progress of the reaction was monitored by LC-MS. After 1 h the reaction mixture was concentrated to 25 mL and extracted with diethyl ether (2×15 mL). The aqueous layer was cooled, acidified to pH 4 with 1 N HCl and extracted with ethyl acetate. The organic layer was dried over Na₂SO₄, filtered and concentrated in vacuo to give a light orange foam, 2,6-bis((tert-butoxycarbonyl)amino)-5-mercaptohexanoic acid (1.80 g, 98%).

¹H NMR (500 MHz, DMSO) δ 12.31 (s, 1H), 7.13-6.90 (m, 2H), 3.81 (s, 1H), 3.01 (m, 2H), 2.76 (s, 1H), 2.31 (d, J=6.7 Hz, 1H), 1.91 (s, 2H), 1.58 (m, 2H), 1.38 (s, 18H). ¹³C NMR (126 MHz, DMSO) δ 174.47, 156.05, 78.46, 78.25, 54.04, 47.92, 32.29, 31.67, 29.06, 28.70. LC-MS: m/z 401.3 [M+Na]⁺, 377.2 [M−H]⁻, rt 10.1 min

To a solution of 2,6-bis((tert-butoxycarbonyl)amino)-5-mercaptohexanoic acid (1.7 g, 4.49 mmol) in dichloromethane (22.5 mL), triethylamine (1.4 mL, 9.88 mmol) and S-methyl methanesulfonothioate (0.85 mL, 8.98 mmol) was added at rt and the reaction mixture was stirred for 30 min. The progress of the reaction was monitored by LC-MS. After 30 min, dichloromethane (100 mL) was added to the reaction mixture and the mixture was washed with water and brine. The organic layer was dried over Na₂SO₄, filtered, concentrated in vacuo and purified by flash column chromatography (methanol:dichloromethne 1:19) to give an off white foam, 20 (1.54 g, 81%).

¹H NMR (500 MHz, CDCl₃) δ 5.34 (d, J=12.0 Hz, 1H), 5.04 (s, 1H), 4.32 (s, 1H), 3.41 (d, J=63.7 Hz, 2H), 2.98-2.74 (m, 1H), 2.43 (s, 3H), 2.27-1.60 (m, 4H), 1.47 (s, 18H). ¹³C NMR (126 MHz, CDCl₃) δ 174.30, 155.92, 155.47, 79.52, 79.34, 53.32, 51.43, 43.34, 40.22, 30.03, 28.75, 24.09. LC-MS: m/z 447.2[M+Na]⁺, 423.1 [M−H]⁻, rt 10.7 min

### 2-Amino-6-((tert-butoxycarbonyl)amino)-5-(methyldisulfanyl)hexanoc acid (11)

Trifluoroacetic acid (3.45 mL, 44.8 mmol) was added to the solution of 20 (1.9 g, 4.48 mmol) in dichloromethane (25 mL). The reaction mixture was stirred at rt for 2 h and the progress of the reaction was monitored by LC-MS. After 2 h the reaction mixture was concentrated in vacuo to yield the TFA salt of 2,6-diamino-5-(methyldisulfanyl)hexanoic acid which was directly used for the next step.

To the TFA salt of 2,6-diamino-5-(methyldisulfanyl)hexanoic acid (2 g, 4.42 mmol) in water (5 mL), sodium bicarbonate (2.62 g, 31.2 mmol) and a solution of CuSO₄.5H₂O (0.568 g, 2.27 mmol) in water (5 mL) was added and stirred vigorously. To the reaction mixture, a solution of di-tert-butyl dicarbonate (1.31 g, 6.02 mmol) in acetone (6 mL) was added and the reaction mixture was stirred for 16 h at rt. The precipitated solid was filtered and washed with water and acetone. The solid was suspended in water (50 mL) and 8-hydroxy quinoline (0.78 g, 5.35 mmol) was added. The reaction mixture was stirred for 16 h, filtered and the green solid residue was washed with water (50 mL). The combined aqueous filtrate was extracted with chloroform (20 mL) and lyophilized to yield pure 11 (0.8 g, 2.5 mmol, 55% over three steps).

¹H NMR (500 MHz, DMSO) δ 8.22 (s, 2H), 6.98 (dd, J=12.5, 6.4 Hz, 1H), 3.84 (q, J=6.4 Hz, 1H), 3.19 (m, 2H), 2.83 (s, 1H), 2.41 (s, 3H), 2.02 (m, 1H), 1.91-1.60 (m, 3H), 1.40 (s, 9H). ¹³C NMR (126 MHz, CDCl₃) δ 171.28, 156.11, 78.36, 52.58, 51.22, 44.14, 28.71, 28.29, 26.99, 24.17. LC-MS: m/z 325.2 [M+H]⁺, 323.1 [M−H]⁻, rt 8.3 min

### Synthesis of Amino Acids 13 and 14

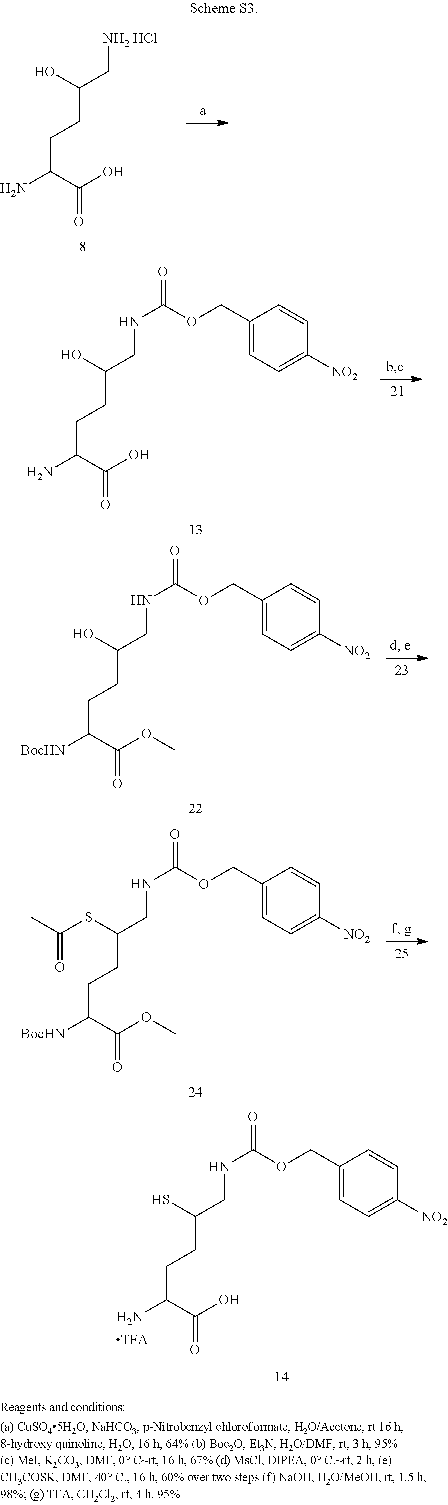

### 2-Amino-5-hydroxy-6-((((4-nitrobenzyl)oxy)carbonyl)amino)hexanoic acid (13)

Na₂HCO₃ (16.93 g, 201 mmol), was added to a solution of 8 (10 g, 50.4 mmol) in water (40 mL) followed by addition of a CuSO₄5H₂O (6.41 g, 25.7 mmol) solution in water (40 mL). To the stirred reaction mixture a solution of 4-nitrobenzyl carbonochloridate (16.29 g, 76 mmol) in acetone (50 mL) was added drop-wise. The reaction mixture was stirred at rt for 16 h. The precipitated solid was filtered through sintered funnel and washed with water. The solid was transferred into a round bottom flask followed by addition of water (500 mL) and powdered 8-hydroxyquinoline (8.77 g, 60.4 mmol). The reaction mixture was vigorously stirred at rt for 16 h and the precipitated green solid was filtered and thoroughly washed with water (2 L). The combined filtrates were extracted by EtOAc (3×250 mL) and concentrated in vacuo to yield 13 as a white solid (11 g, 64%)

¹H NMR (400 MHz, D₂O) δ 8.29 (d, J=9.1 Hz, 1H), 7.63 (d, J=8.9 Hz, 1H), 5.27 (s, 1H), 3.87-3.71 (m, 1H), 3.40-3.11 (m, 11H), 2.00 (ddd, J=16.3, 10.1, 4.9 Hz, 1H), 1.76-1.32 (m, 1H). ¹³C NMR (101 MHz, DMSO) δ 170.02, 155.92, 147.02, 145.18, 128.02, 123.51, 68.93, 63.96, 54.23, 46.77, 30.38, 27.54. LCMS: m/342.20 (M+H)⁺

### 2-((Tert-butoxycarbonyl)amino)-5-hydroxy-6-((((4-nltrobenzyl)oxy)carbonyl)amino)hexanoic acid (21)

Triethylamine (3.18 mL, 22.81 mmol) was added to a solution of 13 (5.56 g, 16.29 mmol) in water (85 mL). A solution of Boc₂O (4.27 g, 19.55 mmol) in DMF (85 mL) was added drop-wise to the above solution and the reaction mixture was stirred at rt. After 3 h reaction mixture was concentrated in vacuo up to its half volume and partitioned between ethyl acetate and water. The pH of water layer was adjusted around 4 and the ethyl acetate layer was washed with water (3×100 mL). The organic layer was dried on anhydrous Na₂SO₄ and concentrated in vacuo to yield 21 (6.8 g, 95%)

¹H NMR (400 MHz, DMSO) δ 8.24 (d, J=8.7 Hz, 2H), 7.61 (d, J=8.6 Hz, 2H), 7.38-7.21 (m, 1H), 6.83 (dd, J=16.5, 7.7 Hz, 1H), 5.17 (s, 2H), 3.89-3.66 (m, 1H), 3.55-3.33 (m, 1H), 2.97 (t, J=5.3 Hz, 2H), 1.76 (m, 2H), 1.56-1.20 (m, 11H). ¹³C NMR (101 MHz, DMSO) δ 174.25, 155.52, 155.83, 147.34, 145.28, 128.01, 123.48, 77.79, 68.66, 63.96, 53.72, 46.52, 36.02, 30.73, 28.16. LCMS: m/342.20 (M-Boc)⁺441.20 (M−H)⁻

### Methyl-2-((tert-butoxycarbonyl)amino)-5-hydroxy-6-((((4-nitrobenzyl)oxy) carbonyl)amino)hexanoate (22)

To a solution of 21 (15.5 g, 35.1 mmol) in anhydrous DMF (160 mL), anhydrous K₂CO₃ (7.28 g, 52.7 mmol) was added at 0° C. The resulting white suspension was stirred for 5 min and methyl iodide (6.6 mL, 105 mmol) was added drop-wise. The reaction mixture was stirred for 16 h. The reaction mixture was filtered through celite and the filtrate was partitioned between ethyl acetate (500 mL) and distilled water (200 mL). The ethyl acetate layer was further extracted with water (3×300 mL), dried over Na₂SO₄, filtered and concentrated in vacuo to give a pale yellow viscous oil, which was purified by flash column chromatography using ethyl acetate:hexane (1: 1) to yield 22 as an oil (10.7 g, 67%)

¹H NMR (400 MHz, CDCl₃) δ 820 (d, J=8.6 Hz, 2H), 7.50 (d, J=8.5 Hz, 2H), 5.19 (s, 3H), 3.73 (d, J=1.5 Hz, 4H), 3.46-3.26 (m, 1H), 3.18-2.99 (m, 1H), 2.11-1.65 (m, 3H), 1.65-1.33 (m, 12H). ¹³C NMR (101 MHz, CDCl₃) δ 173.09, 172.97, 156.63, 155.61, 147.64, 143.88, 128.14, 123.76, 80.38, 70.55, 65.34, 52.83, 52.42, 46.98, 30.17, 30.03, 28.29. LCMS: m/z 356.20 (M-Boc)⁺

### Methyl-5-(acetylthio)-2-((tert-butoxycarbonyl)amino)-6-((((4-nitrobenzyl)oxy) carbonyl)amino)hexanoate (24)

To a solution of 22 (12.3 g, 27 mmol) in dichloromethane (120 mL), N,N-diisopropylethylamine (DIPEA) (9.43 mL, 54 mmol) was added. The solution was cooled to 0° C. and methanesulfonyl chloride (2.57 mL, 32.9 mmol) was added drop-wise. Reaction mixture was stirred at 0° C. for 1 h and was allowed to reach room temperature and stirred for 1 h. The reaction mixture was diluted with dichloromethane (100 mL) and washed with saturated ammonium chloride solution and brine. The organic layer was dried over Na₂SO₄, filtered and concentrated in vacuo to give methyl 2-((tert-butoxycarbonyl)amino)-5-((methylsulfonyl)oxy)-6-((((4-nitrobenzyl)oxy)carbonyl)amino)hexanoate (23) as a light brown foam which was directly used for the next step.

23 (14 g, 26.2 mmol) was dissolved in DMF (525 mL) and potassium thioacetate (9 g, 79 mmol) was added. The reaction mixture was stirred at 40° C. for 16 h and allowed to reach room temperature. Water (250 mL) was added to the reaction mixture and the combined mixture was extracted with ethyl acetate (3×250 mL). The organic layer was washed with water and brine, dried over Na₂SO₄, filtered and concentrated in vacuo to give light brown oil. The crude compound was purified by flash column chromatography using ethyl acetate:hexane (1:2) to yield 24 (8.1 g, 60%).

¹H NMR (400 MHz, CDC₃) δ8.19 (d, J=−8.5 Hz, 2H), 7.48 (d, J=8.4 Hz, 2H), 5.33-5.02 (m, 4H), 4.24 (s, 1H), 3.72 (s, 3H), 3.58 (dd, J=12.7, 7.3 Hz, 1H), 3.49-3.22 (m, 2H), 2.32 (s, 3H), 2.00-1.50 (m, 4H), 1.41 (s, 9H). ¹³C NMR (101 MHz, CDCl₃) δ 195.19, 172.84, 172.76, 171.12, 156.06, 155.52, 147.63, 143.89, 128.15, 123.74, 80.02, 65.32, 60.36, 53.23, 52.89, 52.38, 45.01, 44.83, 44.58, 44.09, 30.80, 29.96, 28.29, 27.70, 27.49, 21.02, 14.18. LCMS: m/414.10 (M-Boc)⁺

### 2-((Tert-butoxycarbonyl)amino)-5-merapto-6-((((4-nitrobenzyl)oxy)carbonyl) amino)hexanoic add (25)

To a solution of 24 (3.25 g, 6.33 mmol) in methanol (30 mL), a degassed solution of NaOH (0.759 g, 18.99 mmol) in water (30 mL) was added drop-wise at 0-5° C. and then the reaction mixture was stirred at rt. The progress of the reaction was monitored by LCMS. After 1.5 h the reaction mixture was concentrated up to 30 mL and extracted with diethyl ether (2×15 mL). The aqueous layer was cooled, acidified with 1N HCl and extracted by ethyl acetate. The organic layer was dried over Na₂SO₄, filtered and concentrated in vacuo to give light orange foam, 25 (2.84 g, 98%).

¹H NMR (400 MHz, DMSO) δ 12.33 (s, 1H), 8.25 (d, J=8.7 Hz, 2H), 7.61 (d, J=8.5 Hz, 3H), 7.04 (t, J=8.1 Hz, 1H), 5.19 (s, 2H), 3.84 (s, 1H), 3.44-3.11 (m, 5H), 2.82 (d, J=6.3 Hz, 1H), 1.84-1.26 (m, 12H). ¹³C NMR (101 MHz, DMSO) δ 172.33, 156.51, 155.80, 146.73, 145.05, 127.86, 123.49, 77.98, 63.82, 59.12, 31.77, 28.16, 21.36, 20.72. LCMS: m/358.20 (M-Boc)⁺

### 1-Carboxy-4-mercapto-5-((((4-ntrobeazyl)oxy)carbonyl)amino)pentan-1-aminlum 2,2,2-trifluoroacetate (14)

Trifluoroacetic acid (3.45 mL, 44.8 mmol) was added to the solution of 25 (4.1 g, 8.96 mmol) in dichloromethane (60 mL). The reaction mixture was stirred at rt and the progress of the reaction was monitored by LCMS. After 4 h reaction mixture was concentrated in vacuo and crystallized with diethyl ether to yield 14 (4 g, 95%).

¹H NMR (400 MHz, D₂O) δ 8.05 (m, 2H), 7.42 (d, 2H), 5.09 (m, 2H), 3.99 (t, J=6.2 Hz, 1H), 3.59-3.06 (m, 2H), 2.87 (s, 1H), 2.31-1.18 (m, 4H). ¹³C NMR (101 MHz, D₂O) δ 172.18, 158.36, 147.23, 145.23, 127.35, 123.67, 112.17, 65.99, 52.59, 47.49, 39.72. LCMS: m/, 358.15 (M+H)*

### Expression of UbδSHK6-HIs₆

50 μL of chemically competent BL21(DE3) cells (Merck Biosciences) were transformed with pBK-nitroCbzKRS* and pCDF-pylT-UbTAG6-His₆ (spectinomycin resistant plasmid containing constitutive PylT and inducible C-terminally His-tagged ubiquitin gene with amber codon at position 6). SOC medium (250 μL) was then added and the cells were incubated at 37° C. for 1 h. LB medium (100 mL) containing spectinomycin (50 μg mL⁻¹) and kanamycin (50 μg mL⁻¹) was then inoculated with the recovered cells (200 μL). After overnight growth, LB medium (500 mL) containing spectinomycin (25 μg mL⁻¹) and kanamycin (25 μg mL⁻¹) was inoculated with the overnight culture (25 mL). Cells were incubated at 37° C. until an OD₆₀₀ of 0.9 was reached. Amino acid 14 (0.23 g) was added directly to the culture and 20 minutes later the cells were induced by the addition of isopropyl-βD-thiogalactopyranoside to 0.5 mM. After expression for 4 h the cells were harvested by centrifugation at 7000 rpm for 10 min.

### Protein Purification

Cells were suspended in 25 mL BugBuster® Protein Extraction Reagent (Merck Biosciences) supplemented with 2-mercaptoethanol (5 mM). The suspension was incubated at 22° C. for 20 min and then clarified by centrifugation at 4° C. (16000×g). The soluble fraction was then incubated with Ni-NTA resin (200 μL) (QIAGEN) for 1 h at 4° C. The slurry was then transferred to an empty column and washed under gravity with 50 mL wash buffer (20 mM Na₂HPO₄ pH 7.4, 25 mM imidazole, 1 mM 2-mercaptoethanol). Protein was then eluted with elution buffer (20 mM Na₂HPO₄ pH 7.4, 300 mM imidazole, 1 mM 2-mercaptoethanol) and collected in 1 mL fractions. Fractions containing protein were determined by SDS-PAGE.

Removal of 1-butyloxycarbonyl Group from Ub6SHK6-His₆

Freeze dried protein was dissolved at a concentration of 4 mg mL⁻¹ in 60% aqueous TFA (250 μL) and incubated at 22° C. for 1 h. Protein was then precipitated by adding ice cold ether (2.5 mL). Protein was collected by centrifugation, solvent removed and the protein air-dried.

### Thiazolidine Ring Opening of UbThzK6-His

Fractions obtained from Ni-NTA purification (3 mL) were combined and supplemented with methoxylamine (200 mM) by the addition of a 6 M aqueous stock solution (100 μL) and 2-mercaptoethanol (1 mM). The pH was then adjusted by the careful addition of 5 M HCl. The solution was then incubated overnight at 25° C. with gentle agitation. The protein was then desalted into H₂O with a PD-10 column (GE Life Sciences) and lyophilized.

Native Chemical Ligation with Ub-MES Thioester

UbδSHK6-His₆ (1.8 mg, 191 nmol) was dissolved in 100 μL ligation buffer (200 mM Na₂HPO₄ pH 7.6, 6 M GdnCl, 100 mM MESNa, 60 mM TCEP). In parallel, Ub-MES thioester (2.5 mg, 287 nmol), prepared as previously described⁴ was dissolved in ligation buffer (100 μL) and the solutions were combined and ligation left to proceed for 48 h at 25° C. The reaction was then reduced by the addition of 1M TCEP dissolved in 4 M NaOH (8 μL). The protein solution was then diluted to ˜0.5 mg mL⁻¹ by the addition of buffer (200 mM NazHPO₄ pH 7.5, 6 M GdnCl). All protein species were then folded by overnight dialysis against phosphate buffered saline (PBS) supplemented with 1 mM dithiothreitol (DTT). Protein was then dialyzed against ion exchange (IEX) buffer A (50 mM ammonium acetate pH 5, 1 mM 2-mercaptoethanol) using a 3.5 kDa MWCO Slide-A-Lyzer dialysis cassette (Thermo Scientific). The ligation product was then purified from residual monoubiquitin by ion exhange (IEX) chromatography using a MonoS column (GE Life Sciences) and a AKTA FPLC system. A gradient running from IEX buffer A to 100% IEX buffer B (50 mM ammonium acetate pH 5, 1M NaCl, 1 mM 2-mercaptoethanol) was applied over 20 min at a flow rate of 2 mL min⁻¹. Fractions containing diubiquitin (0.8 mg, 45 nmol) were determined by SDS-PAGE (FIG. S8). Pooled fractions were dialyzed against degassed buffer (50 mM ammonium acetate pH 5) and protein was concentrated with a centrifugal evaporator (Scanlaf) under reduced pressure to 1 mg mL⁻¹. Protein was then dialyzed overnight against degassed desulfurization buffer (200 mM Na₂HPO₄ pH 7, 0.5 mM TCEP).

### Desulfurization

800 μL of a 1 mg mL⁻¹ solution of undesulfurized K6-linked diubiquitin in desulfurization buffer was mixed with 267 μL of neutralized 1 M TCEP solution, 75 μL 2-dimethly-2-propanethiol and 13 μL of a 0.2 M aqueous solution of VA-044, (2,2′-azobis[2-(2-imidazolin-2-yl)propane]dihydrochloride) as radical intiator. All solutions were prepared immediately prior to use and were extensively purged with argon. The reaction was stirred at 37° C. and desulfurization was complete after 1 h as determined by LC-MS yielding a native isopeptide linkage between the ubiquitin molecules (FIG. 3 and Supplemental FIG. 3) Desulfurization reagents were removed and concomitant folding was achieved by dialysis of the reaction mixture against 10 mM Tris pH 7.6 buffer.

### Deubiquitinase Assay

3 μg of diubiquitin (i75 μmol) was added to 3 μL 10×DUB buffer (500 mM Tris pH 7.5, 500 mM NaCl, 50 mM DTT) and constituted to 20 μL with H₂O. The desired DUB was made up to 10 μL with DUB activation buffer (25 mM Tris pH 7.5, 150 mM NaCl, 10 mM DTT) and incubated at 23° C. for 10 minutes. The DUB was then added to the diubiquitin and the mixture incubated at 37° C. Aliquots of the reaction (6 μL) were quenched by addition of 4×SDS sample buffer (6 μL) and loaded on to an 4-12% SDS-PAGE gel to resolve diubiquitin from monoubiquitin (Ub), resulting from deubiquitinase-mediated cleavage. For the USP2 and USP5 DUBs (ENZO LifeSciences) we used 0.2 μg (4.8 pmol, 2.1 pmol respectively) of enzyme per reaction. Reactions also included 0.2 μg UCH-L3^{[4]} (8 pmol) to remove the His-tag. Staining was carried out with the Silver Stain Plus kit (Bio-Rad) in accordance with the manufacturers instructions.
(1) Cropp, T.; Anderson, J.; Chin, J. Nat Protoc 2007, 2, 2590-600.
(2) Neumann, H.; Peak-Chew, S.; Chin, J. Nat Chems Biol 2008.
(3) Gautier, A.; Nguyen, D. P.; Lusic, H.; An, W.; Deiters, A.; Chin, J. W. Journal of the American Chemical Society 2010, 132, 4086-8.
(4) Virdee, S.; Ye, Y.; Nguyen, D. P.; Komander, D.; Chin, J. W. Nat CheaM Biol 2010, 6, 750-7.

1. A tRNA synthetase capable of binding delta-substituted lysine,
wherein said tRNA synthetase comprises amino acid sequence corresponding to the amino acid sequence of at least L271 to Y349 of MbPyIRS,
wherein said sequence comprises 5 or fewer substitutions within the amino acid sequence corresponding to the amino acid sequence of at least L271 to Y349 of MbPyIRS; and
wherein said synthetase comprises W at amino acid position 349 relative to MbPyIRS.

2. A tRNA synthetase according to claim 1 wherein the tRNA synthetase comprises N at position 311.

3. A tRNA synthetase according to claim 1 wherein the tRNA synthetase further comprises a mutation relative to the wild type MbPyIRS sequence at one or more of Y271, L274 and C313.

4. A tRNA synthetase according to claim 3 which comprises Y271M, L274G and C313A.

5. A nucleic acid comprising nucleotide sequence encoding a tRNA synthetase according to claim 1.

6-7. (canceled)

8. A method of making a polypeptide comprising delta-substituted lysine comprising arranging for the translation of a RNA encoding said polypeptide, wherein said RNA comprises an orthogonal codon, wherein said translation is carried out in the presence of a tRNA synthetase according to claim 1 and in the presence of tRNA which recognises the orthogonal codon and is capable of being charged with delta-substituted lysine, and in the presence of delta-substituted lysine.

9. A method according to claim 8 wherein the orthogonal codon is the amber codon (TAG).

10. A method according to claim 8 wherein the delta-substituted lysine is also epsilon substituted.

11. A method according to claim 8 wherein the delta-substituted lysine is selected from the group consisting of

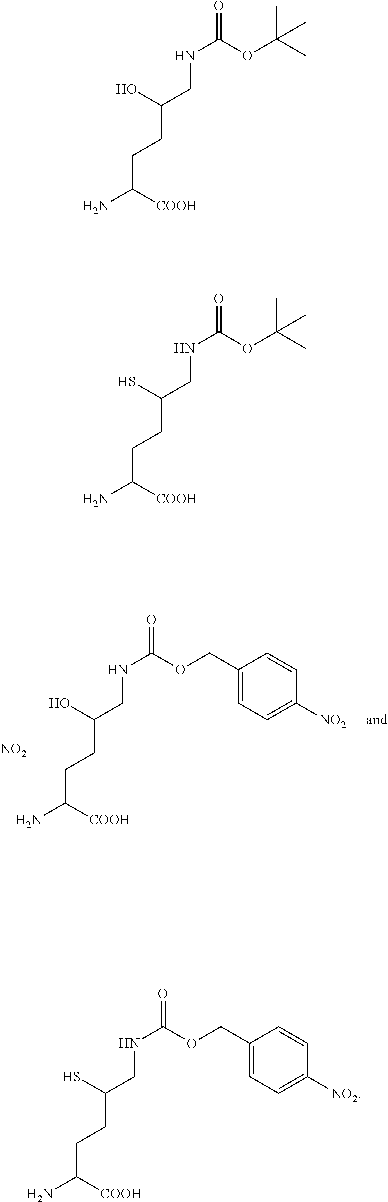 [SMILES: CC(C)(C)OC(=O)NCC(O)CCC(N)C(=O)O ; CC(C)(C)OC(=O)NCC(S)CCC(N)C(=O)O ; "NO2" ; NC(CCC(O)CNC(=O)OCc1ccc([N+](=O)[O-])cc1)C(=O)O ; NC(CCC(S)CNC(=O)OCc1ccc([N+](=O)[O-])cc1)C(=O)O]

12. A method according to claim 11 wherein the delta-substituted lysine is 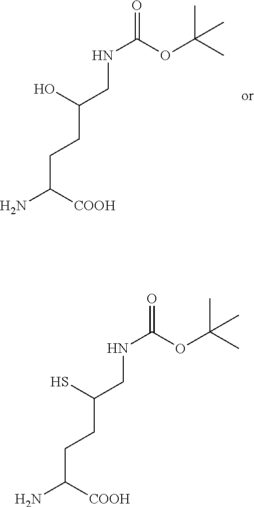 [SMILES: CC(C)(C)OC(=O)NCC(O)CCC(N)C(=O)O ; CC(C)(C)OC(=O)NCC(S)CCC(N)C(=O)O]
and wherein the method further comprises the step of removing the butyloxycarbonyl (boc) group.

13. A method according to claim 12 wherein the step of removing the butyloxycarbonyl (boc) group comprises contacting the polypeptide with 60% trifluoroacetic acid (TFA) at 22° C. for 1 hour.

14. A method according to claim 11 wherein the delta-substituted lysine is 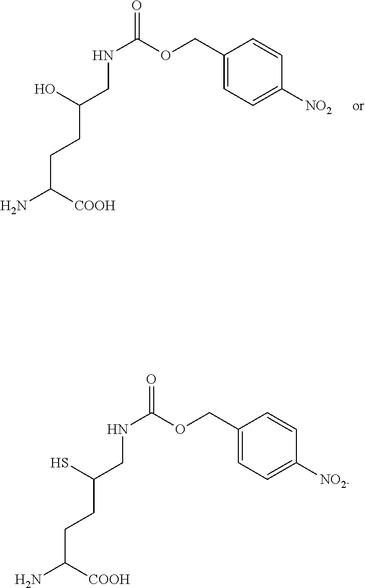 [SMILES: NC(CCC(O)CNC(=O)OCc1ccc([N+](=O)[O-])cc1)C(=O)O ; NC(CCC(S)CNC(=O)OCc1ccc([N+](=O)[O-])cc1)C(=O)O]
and wherein the method further comprises the step of removing the nitrocarbylbenzyloxy (nitroCbz) group.

15. A method according to claim 14 wherein the step of removing the nitrocarbylbenzyloxy (nitroCbz) group comprises reducing the aromatic nitro group to aniline and fragmenting the aniline to reveal the free epsilon amino group.

16. A method according to claim 14 wherein the step of removing the nitrocarbylbenzyloxy (nitroCbz) group comprises performing one-fix-elimination.

17. A method of incorporating a ubiquitin-like modifier into a polypeptide comprising
(a) incorporating a delta-substituted lysine into a polypeptide according to claim 8; and
(b) ligating said ubiquitin-like modifier to the delta-substituted lysine of (a).

18. A method according to claim 17 wherein the ubiquitin-like modifier comprises ubiquitin, SUMO, ISG15, Nedd, FAT10, Ufm1 or ATG12.

19. A method according to claim 18 wherein the ubiquitin-like modifier comprises ubiquitin.

20. A delta-substituted lysine selected from the group consisting of

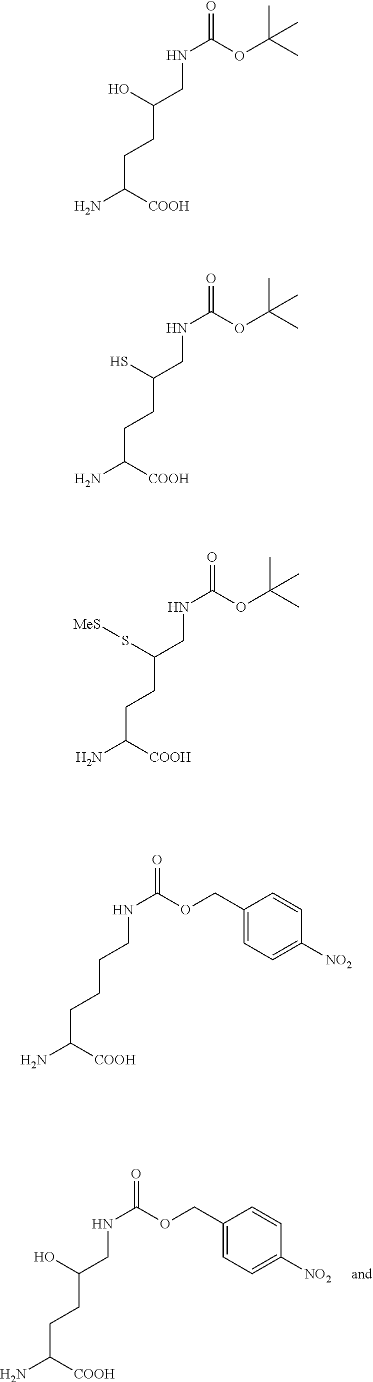 [SMILES: CC(C)(C)OC(=O)NCC(O)CCC(N)C(=O)O ; CC(C)(C)OC(=O)NCC(S)CCC(N)C(=O)O ; CSSC(CCC(N)C(=O)O)CNC(=O)OC(C)(C)C ; NC(CCCCNC(=O)OCc1ccc([N+](=O)[O-])cc1)C(=O)O ; NC(CCC(O)CNC(=O)OCc1ccc([N+](=O)[O-])cc1)C(=O)O]

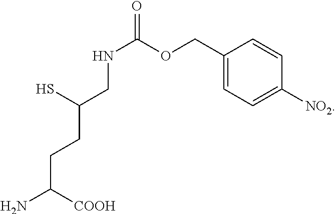 [SMILES: NC(CCC(S)CNC(=O)OCc1ccc([N+](=O)[O-])cc1)C(=O)O]

21. A polypeptide comprising a delta-substituted lysine according to claim 20.

22. A method according to claim 8 wherein the lysine is an isotopically labelled lysine.

23. A vector comprising nucleic acid according to claim 5.

24. A vector according to claim 23, said vector further comprising nucleic acid sequence encoding a tRNA substrate of said tRNA synthetase.

25. A vector according to claim 24 wherein said tRNA substrate is encoded by the MbPy1T gene.

26. A cell comprising a nucleic acid according to claim 5.

27. A kit comprising
(i) a vector according to claim 23; and
(ii) a delta substituted lysine selected from the group consisting of

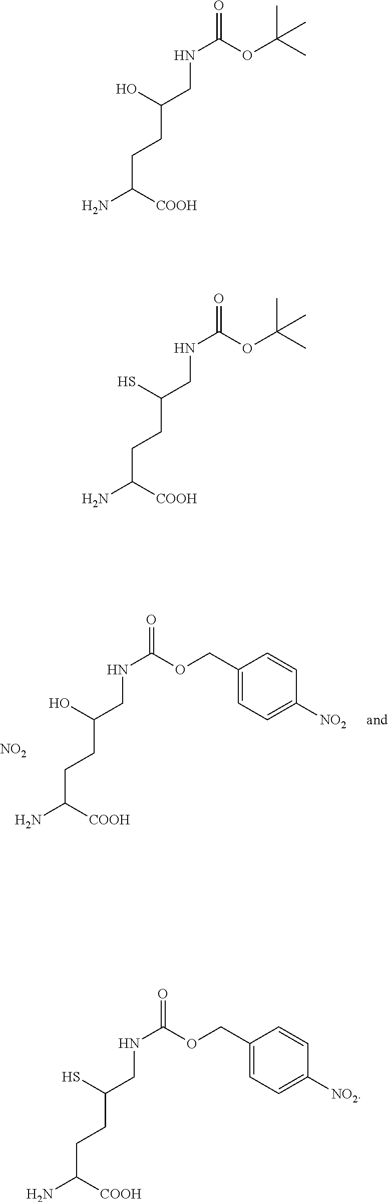 [SMILES: CC(C)(C)OC(=O)NCC(O)CCC(N)C(=O)O ; CC(C)(C)OC(=O)NCC(S)CCC(N)C(=O)O ; "NO2" ; NC(CCC(O)CNC(=O)OCc1ccc([N+](=O)[O-])cc1)C(=O)O ; NC(CCC(S)CNC(=O)OCc1ccc([N+](=O)[O-])cc1)C(=O)O]

28. A kit according to claim 27 further comprising
(iii) a vector comprising sequence encoding the MbPylT tRNA.

29. A kit according to claim 28 wherein the vector of (iii) further comprises a cloning site to accept nucleic acid sequence encoding the target polypeptide and further comprises nucleic acid elements capable of directing expression of said target polypeptide.

30. (canceled)
